(19) United States
(12) Patent Application Publication
(10) Pub. No.: US 2023/0203586 A1
(43) Pub. Date: Jun. 29, 2023

(54) METHOD AND SYSTEM FOR RNA ISOLATION FROM SELF-COLLECTED AND SMALL VOLUME SAMPLES

(71) Applicant: The Rockefeller University, New York, NY (US)

(72) Inventors: Robert B. Darnell, New York, NY (US); Dana Orange, New York, NY (US)

(21) Appl. No.: 17/928,424

(22) PCT Filed: May 28, 2021
(86) PCT No.: PCT/US2021/034785

§ 371 Date: Nov. 29, 2022

Related U.S. Application Data
(60) Provisional application No. 63/031,861, filed on May 29, 2020.
(60) Provisional application No. 63/050,155, filed on Jul. 10, 2020.
(60) Provisional application No. 63/135,159, filed on Jan. 8, 2021.
(60) Provisional application No. 63/135,224, filed on Jan. 8, 2021.
(60) Provisional application No. 63/171,749, filed on Apr. 7, 2021.

(51) Int. Cl. C12Q 1/6883

(52) U.S. Cl. CPC C12Q 1/6883; C12Q 1/6806

(57) ABSTRACT

The present invention provides methods for isolation and characterization of nucleic acid, particularly RNA, from small volume and self-collected samples, including fingerstick blood samples, swabs and saliva samples. The RNA derived is intact and of sufficient quality and quantity for RNA analysis, longitudinal RNA sequencing and global transcriptomic profiling.

### FIELD OF THE INVENTION

The present invention relates generally to the isolation and characterization of nucleic acid, particularly RNA, from small volume and self-collected samples, including fingerstick blood samples, swabs and saliva samples. The RNA derived is intact and of sufficient quality and quantity for RNA analysis, longitudinal RNA sequencing and global transcriptomic profiling.

### BACKGROUND OF THE INVENTION

RNA analysis, longitudinal RNA sequencing and global transcriptomic profiling are useful tools to identify and analyze biomarkers for disease, infection, exposure, susceptibility, drug response and toxicity (Frank M O et al (2019) BMC Medical Genetics 12:56; doi.org/10.1186/s12920-019-0500-0; Casamassimi A et al (2017) Int J Mol Sci 18(8):1652; Sheid A D et al (2018) J Immunol 200:1817-1928). In some disease and cancer studies, solid tissues or tumor samples are utilized, however, this is not practical in clinical studies or for continuous monitoring. Peripheral blood has advantages for biomarker evaluation and discovery due to its non-invasive collection and availability, particularly in comparison to solid tissue samples. A number of studies have demonstrated that transcriptomic changes in peripheral blood can serve as biomarkers of infection, exposure to xenobiotics, response to therapeutics or vaccines, or as indicators for pathological changes occurring in other tissues (Bushel P R et al (2007) Proc Natl Acad Sci 104(46):18211-6; Ramilo O et al (2007) Blood 109:2066-2077; Mejias A et al (2013) PLoSMed 10(11):e1001549; Hecker Met al (2013) Molec Neurobiol 48:737-756; Querec T D et al (2009) Nat Immunol 10:116-125).

The use of whole blood for transcriptomic profiling and RNA evaluation presents a number of significant technical challenges, including that RNA degradation and transcriptomic changes can occur quickly after the blood is drawn from subjects. Traditional reagents, such as citrate salts, heparin, and EDTA, inhibit blood clotting, but do not stabilize mRNA transcripts and altered gene regulation has been observed in whole blood samples, particularly when RNA is not immediately isolated (Debey S et al (2004) Pharmacogenomics 4:193-207; Rainen L et al (2002) Clin Chem 48:1883-1890). Moreover, the majority of RNA from whole blood encodes the globin protein, and sequencing that does not take this into consideration can yield results of low complexity (ie mostly globin mRNA, and few other unique or rare mRNA species).

Approaches for blood RNA stabilization have been developed to address the issues for RNA analysis from whole and peripheral blood samples (Asare A L et al (2008) BMC Genomics 9:474; Rainen L (2002) Clin Chem 48:1883-1890; Chai V et al (2005) J Clin Lab Anal 19_182-188). These include the PAXgene™ Blood RNA system (Qiagen) and the Tempus™ system (Applied Biosystems). In both systems, blood is immediately lysed when collected into the tube and RNA is stabilized using specific reagents. The PAX blood collection tube system and stabilization buffer is described including in U.S. Pat. Nos. 6,602,718 and 6,617,170. Aspects of the Tempus guanidinium-based stabilization agent are provided in U.S. Pat. No. 5,972,613. Both of these systems are designed for and require 2.5 mls or 3 mls of whole blood, which necessitates venipuncture, and are not suitable for small blood samples such as from a laboratory animal, an infant, or for any applicable means of self-collection.

Fingerstick blood collection is a practical and minimally invasive sample collection method that is used for a wide range of applications in routine clinical practice and can be implemented outside of clinical settings. For example, fingerstick sampling is used by millions of individuals to collect daily small blood volumes to monitor sugar or glucose levels. Finger stick blood collection would also be of value in subjects where it is commonly difficult to collect blood via venipuncture such as in infants and young children, elderly or ill individuals with compromised veins, intravenous drug addicts, and very obese individuals, in field studies in remote and under-developed areas, in military subjects or physically active athletes, or in other situations such as where a rapid sample is necessitated or applicable or in instances where collection of a large number of samples, including from many individuals, need to be obtained in a short amount of time.

The RNA collection and analysis systems currently in use and available, however, are not designed for small volume samples, such as finger stick blood samples or samples of one or a few droplets of blood. Collection of small volumes of blood via finger sticks is especially indicated for high frequency or repeated sample collection, such as to enable monitoring individuals in health and disease or infection. In addition, these systems are not applicable for alternative types of samples which may be time and sample volume critical such as naspharyngeal, nasal or throat swabs or aspirates. These are commonly utilized in direct and rapid patient assessment for virus infection, particularly respiratory virus infection, such as for influenza, so that infection can be quickly evaluated and treatment prescribed.

In order to implement and apply RNA isolation, evaluation and analysis more broadly and across various clinical and nonclinical scenarios and situations, there is a need for methods and a system to reliably and effectively sample and analyze RNA from small volume samples and alternative sample types that can be collected frequently, rapidly, in large number, in the field or at home by a relatively untrained individual or non-health professional or patient. There is a need for straightforward and dependable systems and methods whereby RNA can be isolated from small volume samples, self-collection samples, fingerstick samples and evaluated qualitatively and quantitatively with confidence and dependable results, particularly for whole transcriptome analysis and profiling.

### SUMMARY OF THE INVENTION

The present invention general relates to methods for RNA isolation and RNA profiling and analysis of small volume samples and self-collected samples, wherein the RNA is of sufficient quality and quantity for whole transcriptome analysis and transcriptomic profiling. In embodiments of the method, a small volume sample may be from a patient or individual having a disease or infection or at risk for or suspected of disease or infection. In some embodiments, the patient or individual obtains or collects the small volume sample. In some embodiments, the patient or individual is assisted by a non-medical person in collection of the sample. In an embodiment, the sample is collected from a patient or individual by a non-medical person, such as a spouse, parent, friend, guardian, etc that is not medically trained or involved in any medical profession. Critically, the invention describes methods to obtain sufficient quality and quantity of RNA for a variety of analyses, ranging from quantitating individual RNA species to sequencing entire transcriptomes of high complexity.

In accordance with the method, small volume sample(s) is collected and combined with an RNA stabilization solution. In some embodiments, the RNA stabilization solution is capable of lysing the cells in the sample and of stabilizing RNA contained in the cells or cell lysate of the sample. In some embodiments, the RNA stabilization solution is capable of lysing the cells in the sample and of stabilizing RNA contained in the cells or cell lysate of the sample in a single step. In embodiments, the sample and RNA stabilization solution are mixed, vortexed or shaken when combined. In some embodiments, the sample may be stored or left at room temperature for up to a few or several hours prior to refrigeration. In some embodiments, the sample is then stored in refrigerated conditions, such as at about 40° F. or about 4° C. for a brief time. In some embodiments, the sample is then stored in refrigerated conditions, such as at about 40° F. or about 4° C. for a brief time, up to a day or a few or several days. In some embodiments, the sample may be stored or left at room temperature for up to a few or several hours, up to 2 hours, up to 3 hours, up to 3 or 4 hours, prior to refrigeration. In some embodiments, the sample is then stored in refrigerated conditions, such as at about 40° F. or about 4° C. for a brief time, up to a day or a few or several days. In some embodiments, the sample is stored in a freezer or in frozen temperature conditions, such as at about 30 or 32° F. or about 0° C., either after collection, after brief (2-4 hour) storage at room temperature, or after brief (1-2 day) refrigerated storage.

A small volume sample may be less than 500 μl, less than 300 μl, less than 250 μl, about 200-300 μl, less than 200 μl, about 100-300 μl, about 150-300 μl, about 100-250 μl, about 50-300 μl. In an embodiment, a small volume sample volume is about 100-300 μl.

In some embodiments, the sample may be the sample is a small volume blood sample, a sputum or saliva sample, or a nasal, nasopharyngeal or oropharyngeal swab, wash or aspirate. In embodiments, the small volume sample is a blood sample and is collected via fingerstick or heelprick. In an embodiment, the small volume sample is a blood sample and is collected via fingerstick. In embodiments, the fingerstick sample or heelprick sample may comprise blood droplets directly from a fingerstick or heelprick or a capillary tube may be utilized.

In some embodiments, the sample volume is less than 500 μl, less than 300 μl, less than 250 μl, about 200-300 μl, less than 200 μl, about 100-300 μl, about 150-300 μl, about 100-250 μl, about 50-300 μl. In some embodiments, the volume is less than 100 μl, less than 50 μl, about 10-50 μl, about 10-20 μl, about 10 μl, as small as 10 μl or less. In an embodiment, the sample volume is about 100-300 μl. In an embodiment, the sample volume is about 50-300 μl. In some embodiments, the sample volume is on the order of a blood droplet volume, or one or a few blood droplet volumes. In some embodiments, the blood or sample volume is that of a capillary tube volume, or less than a blood droplet volume. Capillary tube sample volumes may be on the order of 60-100 μl, 100-200 μl, 5-25 μl, 10-50 μl, less than 10 μl, 1-5 μl.

In some embodiments, the volume of RNA stabilization solution is less than 1 ml, about 500 μl or less, about 300 μl or less, about 200-300 μl, or about 250 μl, or about 200 μl. In an embodiment, the volume of RNA stabilization solution is about 300 μl or less, about 200-300 μl, or about 250 μl, or about 200 μl. In some embodiments, including wherein the sample volume is very low, such as on the order of less than 50 μl, or 10-50 μl, or about 10 μl, or less than 10 μl, the volume of RNA stabilization solution is appropriately low, such as on the order of less than 100 μl, less than 50 μl, less than 25 μl, as small as 10 μl or less.

In some embodiments, the sample is collected into a tube or wherein the tube or receptacle for receiving the small volume sample and containing RNA stabilization solution has a total volume capacity of 1.5 ml or less, 1.2 ml or less, or lml or less, or less than lml, or less than 500 μl, or less than 300 μl, or less than 200 μl. In an embodiment, the sample is collected into a tube or wherein the tube or receptacle for receiving the small volume sample and containing RNA stabilization solution has a total volume capacity of 1.5 ml or less, such as a microtainer tube. In an embodiment, the sample is collected in a tube which is suitable for small volumes, including very small volumes, such as a capillary tube. In an embodiment, the sample is collected into a capillary tube, which is suitable for small volumes, such as less than 100 μl, or even for very small volumes, such as less than 50 μl, less than 25 μl.

The invention provides a method for RNA profiling and analysis of small volume samples from a patient or individual comprising:
(a) obtaining one or more small volume sample self-collected by the patient or individual or by a non-medical person, wherein the sample is collected in or otherwise combined with an RNA stabilization solution whereby cells in the sample are lysed and RNA is stabilized; and
(b) isolating RNA using a process adapted for small volume samples wherein the amount of any and all solutions or buffers utilized are reduced and adjusted for small volume samples;

wherein the RNA is of sufficient quality and quantity for whole transcriptome analysis and transcriptomic profiling through RNA sequencing (RNAseq).

In embodiments of the method, the RNA is isolated using a process comprising:
(a) contacting the sample with a protease to form a protease treated small volume sample;
(b) contacting the protease treated sample with an ethanol or salt solution forming a precipitate containing the RNA, wherein the precipitate containing the RNA is then resuspended in a buffer or solution, or contacting the protease treated sample with an organic extraction solution, forming a solution having an aqueous phase containing the RNA and an organic phase;
(c) contacting the resuspended precipitate containing the RNA or the aqueous phase containing the RNA with DNAse to form a DNAse-treated resuspended precipitate or DNAse-treated aqueous phase;
(d) binding the RNA to a silica based solid phase or column by contacting the resuspended precipitate or aqueous phase with said silica based solid phase; and
(e) eluting the RNA from the silica based solid phase comprising contacting the silica based solid phase with a solution or buffer to provide isolated RNA;
wherein all buffer and solution volumes are reduced and adjusted for small volume samples.

In some embodiments, between steps (b) and (c), the resuspended precipitate containing the RNA or the aqueous phase containing the RNA is contacted with a solution or column to remove residual sample cell debris and/or to homogenize the sample cell lysate.

For embodiments using protease, the protease may be proteinase K. For embodiments using proteinase K, lysis buffers may also contain detergents, which both inactivate adventitious agents, lyse cells, and activate the proteinase K. Proteinase K has activity at 25° C., although it can be activated by putting the collected sample in hot tap water (typical tap water is set at a maximum of 120° F., which is about 48° C.; Proteinase K is optimally active at ˜55° C.).

In embodiments of the method, the RNA is isolated using a process comprising:
(a) contacting the sample with an RNA stabilization solution, wherein the solution has capability to lyse cells and inactivate adventitious agents;
(b) contacting the RNA stabilization solution treated sample with an ethanol or salt solution forming a precipitate containing the RNA, wherein the precipitate containing the RNA is then resuspended in a buffer or solution, or contacting the RNA stabilization solution treated sample with an organic extraction solution, forming a solution having an aqueous phase containing the RNA and an organic phase;
(c) contacting the resuspended precipitate containing the RNA or the aqueous phase containing the RNA with DNAse to form a DNAse-treated resuspended precipitate or DNAse-treated aqueous phase;
(d) binding the RNA to a silica based solid phase or column by contacting the resuspended precipitate or aqueous phase with said silica based solid phase; and
(e) eluting the RNA from the silica based solid phase comprising contacting the silica based solid phase with a solution or buffer to provide isolated RNA;
wherein all buffer and solution volumes are reduced and adjusted for small volume samples.

In embodiments of the method, the RNA is isolated using a process comprising:
(a) contacting the sample with an RNA stabilization solution, wherein the solution has capability to lyse cells and inactivate adventitious agents;
(b) optionally further contacting the sample with a salt, a reducing agent, and/or a detergent;
(c) contacting the solution contacted sample of (a) or (b) with silica, silica based solid phase or carboxylated magnetic beads which bind RNA and seves to purify the RNA from other components in the sample; and
(d) eluting the RNA from the silica or silica based solid phase or the magnetic beads comprising contacting the silica, silica based solid phase or magnetic beads with a solution or buffer to provide isolated RNA;
wherein all buffer and solution volumes are reduced and adjusted for small volume samples.

In an embodiment, the RNA stabilization solution may be a mixture of chaotropic salt and phenol. In an embodiment, the chaotropic salt may be a guanidine salt or guanidine based. In an amboiment, the RNA stabilization soltion may be the PAXgene RNA stabilization solution.

In an embodiment, purification out of chaotropic salts such as guanidine, with detergent, can be used. The downstream purification as in step (c) may be precipitation, contact with a nucleic acid binding solid bead or semi-porous bead, such as a silica or carboxylated magnetic bead. Modification of lysis buffer for contact with silica or magnetic beads may be to include salt (e.g. sodium acetate) detergent (e.g. 0.2% sarkosyl), reducing agent (e.g. dithiothreotol, e.g. 75 mM). Purification may be accomplished using magnets to purify nucleic acids, by washing magnetic beads with bound nucleic acid in 75-80% ethanol or isorpropanol, twice, and then eluting RNA off the magnetic beads in pure RNase free double distilled water (ddH2O).

In some embodiments the sample is a small volume blood sample, a sputum or saliva sample, or a nasal, nasopharyngeal or oropharyngeal swab, wash or aspirate. In some embodiments, the sample is a small volume blood sample. In an embodiment, the small volume sample is a blood sample and is collected via fingerstick. In embodiments, the fingerstick sample may comprise blood droplets directly from a fingerstick or a capillary tube may be utilized.

In some embodiments of the method(s), the sample volume is less than 500 μl, less than 300 μl, less than 250 μl, about 200-300 μl, less than 200 μl, about 100-300 μl, about 150-300 μl, about 100-250 μl, about 50-300 μl. In an embodiment, the sample volume is about 100-300 μl. In some embodiments, the volume is less than 100 μl, less than 50 μl, about 10-50 μl, about 10-20 μl, about 10 μl, as small as 10 μl or less. In an embodiment, the sample volume is about 50-300 μl. In an embodiment, the sample volume is about 50-250 μl or is about 50-200 μl.

In some embodiments of the method(s), buffer and solution volumes are reduced to 20-40% or 20-30% of those utilized for isolation of RNA from a standard venipuncture blood sample.

In some embodiments, the RNA stabilization solution is a chaotropic salt such as guanidinium thiocyanate based or containing solution. In some embodiments chaotropic salts such as guanidinium thiocyanate based lysis buffers may also contain detergents, which synergize to inactivate adventitious agents, lyse cells. Detergents may include sarkosyl, SDS, or other ionic or non-ionic detergents. Kits/lysis solutions containing chaotropic salts such as guanidinium thiocyanate based lysis buffers with or without detergents, are stable, even up to for years. They can be shipped and used at room temperature. They are less toxic than household bleach, and can be mailed with adherence to suitable or such standards

In some embodiments, any buffers or solutions are made, prepared or generated with RNAse free water or buffers.

In embodiments of the method, any suitable and efficacious protease is utilized. Suitable proteases are known and available in the art. In embodiment, the protease is proteinase K. In some embodiments, the sample is contacted and treated with a protease at a temperature above room temperature. In embodiments, the sample and protease are heated for protease treatment. In an embodiment, the sample and protease are heated to 50-60° C. or incubated at a temperature of 50-60° C. In an embodiment, the sample and protease are heated to or incubated at 55° C.

In accordance with embodiments of the method, the method further comprises sequencing the RNA. RNA may be sequenced using any suitable or recognized method, steps, system(s) or kit(s), including manual, semi-automated or automated method(s), system(s) or kits. In some embodiments, kits such as Illumina TruSeq or Kapa Hyper Prep Kits are utilized.

In an embodiment, the isolated RNA is converted to cDNA. In some embodiments, the isolated RNA is converted to cDNA and may be cloned or a library prepared therefrom or containing or based on the cDNA(s).

In some embodiments, abundant RNA species or RNA species not of interest are removed prior to sequencing. In embodiments, globin mRNA, ribosomal RNA(s) or species specific RNAs are removed prior to sequencing. Methods, systems and kits for removal of globin RNA and/or ribosomal RNA are know and available to one skilled in the art. In some embodiments, systems or kits such as BlobinZero (Illumina), Ribo-Zero Gold, TruSeq Stranded total RNA library prep, Ribo-Zero Globin, GLOBINclear kit (THermo Fisher Scientific), QIAseqFastSelect RNA removal kit (Qiagen) may be utilized. In some embodiments, species specific probes may be utilize to select out certain RNAs.

In embodiments or the method, the patient or individual has a disease or infection or is at risk of or suspected of disease or infection.

In some embodiments, the method is for longitudinal screening by RNA profiling and analysis of small volume samples from one or more patient or individual, wherein the patient or individual has a disease or infection or is at risk of or suspected of disease or infection. In embodiments, small volume samples are collected in series or in regular or designated increments of hours, days, weeks or months. In embodiments, small volume blood samples are collected via fingerstick in series or in regular or designated increments of hours, days, weeks or months.

In some embodiments, small volume samples may be collected or additionally collected at outset of symptom(s), such as one or more symptom or recognized parameter indicative of or associated with a disease or infection. The disease may be an acute or chronic disease. The disease may be a relapsing and/or remitting disease. The infection may be a bacterial or viral infection. The infection may be with a known or unknown infectious agent. The infection may be with a known or unknown virus or bacteria.

In embodiments of the invention, systems and kits for use and application of the methods are provided.

In embodiments, a system or kit is provided for RNA profiling and analysis of small volume samples from a patient or individual comprising:
(a) a means for self-collection of a small volume sample by the patient or individual or by a non-medical person, comprising a lancet, swab or receptable for a wash, spit or aspirate;
(b) a tube or receptacle for receiving the small volume sample on collection and containing a volume of RNA stabilization solution whereby cells in the sample are lysed and RNA is stabilized; and
(c) one or more appropriate label(s) for designating the name or identity of the patient or individual, date of sample collection and time of sample collection.

In embodiments, the system or kit may further comprise an envelope or mailing container for shipment of the sample to a laboratory or facility for RNA isolation and analysis.

In some embodiments, the system or kit may be for longitudinal RNA profiling and analysis of multiple small volume samples collected in series from a patient or individual over days, weeks or months comprising:
(a) a set of numerous means for self-collection of individual small volume samples by the patient or individual or by a non-medical person, each comprising a lancet, swab or receptable for a wash, spit or aspirate;
(b) a set of numerous tubes or receptacles each individually for receiving a small volume sample on collection and containing a volume of RNA stabilization solution whereby cells in the sample are lysed and RNA is stabilized;
(c) numerous appropriate label(s) for designating the name or identity of the patient or individual, date of sample collection and time of sample collection; and
(d) numerous envelopes or mailing containers for shipment of each sample or several samples to a laboratory or facility for RNA isolation and analysis.

In some embodiments of the system or kit, the volume of RNA stabilization solution is less than lml, about 500 μl or less, about 300 μl or less, about 200-300 μl, or about 250 μl. In an embodiment, the volume of RNA stabilization solution is about 300 μl or less, about 200-300 μl, or about 250 μl. In some embodiments, including wherein the sample volume is very low, such as on the order of less than 50 μl, or 10-50 μl, or about 10 μl, or less than 10 μl, the volume of RNA stabilization solution is appropriately low, such as on the order of less than 100 μl, less than 50 μl, less than 2 μl, as small as 10 μl or less

In some embodiments of the system or kit, the tube or receptacle for receiving the small volume sample and containing RNA stabilization solution has a total volume capacity of 1.5 ml or less, 1.2 ml or less, or lml or less. In some embodiments, the tube or receptacle for receiving the small volume sample and containing RNA stabilization solution is a tube which is suitable for small volumes, including very small volumes, such as a capillary tube. In an embodiment, the tube or receptacle is a capillary tube, which is suitable for small volumes, such as less than 100 μl, or even for very small volumes, such as less than 50 μl, less than 25 μl.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

The patent or patent application contains at least one drawing executed in color. Copies of this patent or patent application publication with color drawing(s) will be provided by the U.S. Patent and Trademark Office upon request and payment of the necessary fee.

FIG. 1 depicts the study overview and validation of in-home assessments of disease activity and gene expression. A. Climical data collection and RNA analysis over time. Study overview of clinical data and sample collection over time. B. Clinical and patient reported assessments of disease activity. Correlation between disease activity scores measured in clinic (DAS28) and at home (RAPID3 questionnaire) from the index patient. C. Clinical blood counts and RNASeq-inferred blood counts. Neutrophil, lymphocyte, and monocyte counts measured from paired clinical complete blood counts from venipuncture blood draws and CIBERSORTx inferred blood counts from RNAseq data from finger stick blood draws (N=38 paired samples).

FIG. 2 depicts RNA quality and quantity by volume of fixative. 3 drops of blood harvested with a 21 guage lancet were added to a microtainer tube prefilled with either 250 μl, 500 μl or 750 μl of PAX gene fixative. Samples were stored at room temperature for 3 days and then RNA was extracted using the PAX gene RNA kit and RIN scores and quantity of RNA was assessed using the Agilent 2100 Bioanalyzer picochip. Padj=ANOVA, followed by Dunnett's multiple comparisons test, using 250 μl as the reference group.

FIG. 3 depicts RNA quality and quantity by time at room temperature. 100 μl of whole blood was added to a microtainer tube prefilled with 250 μl PAX gene fixative and frozen after 2 hours, 3 days, or 7 days incubation at room temperature. RNA was extracted using the PAX gene RNA kit with scaled down washes and elutions and RIN scores and quantity of RNA was assessed using the Agilent 2100 BioAnalyzer RNA picochip. Padj=ANOVA, followed by Dunnett's multiple comparisons test, using Day 0 as the reference group.

FIG. 4 depicts RNA quality and quantity of fresh and mailed samples. 100 μl of whole blood was added to a microtainer tube prefilled with 250 μl PAX gene fixative and frozen after 2-hour incubation at room temperature or mailed. RNA was extracted using the PAX gene RNA kit and RIN scores and quantity of RNA was assessed using the Agilent 2100 BioAnalyzer RNA picochip.

FIG. 5 depicts RNA quality and quantity by volume of extraction and washes. 3 drops of blood harvested with a 21 guage lancet were added to a microtainer tube prefilled with 250 μl of PAX gene fixative. Samples were stored at room temperature for 3 days and then RNA was extracted using the PAXgeneRNA kit according to manufacturer's directions or with a scaled down version of the PAX protocol, using approximately 25% of the recommended volumes for all washes and elutions. RIN scores and quantity of RNA was assessed using the Agilent 2100 BioAnalyzerRNA picochip. P=unpaired two-sided t test.

FIG. 6 depicts RNA quality and quantity with and without TriZol reagent extraction step. Mailed patient finger stick samples were stored in PAXgeneRNA buffer at −80° C. 142 samples had RNA extracted with PAXgeneRNA extraction with low volume washes, 13 samples were thawed and mixed with 700 μl Trizol-LS, and 250 μl chloroform. After centrifugation, the top layer was precipitated with isopropanol and glycogen and washed with 80% cold ethanol, centrifuged and the pellet was dried, resuspended in PBS and then purified using the Roche High Pure Isolation kit. P values represent significance of unpaired T tests.

FIG. 7 depicts a comparison of Cycle Times for HbgA2, 18S RNA, and TNF alpha after GlobinZero depletion. Since ribosomal and hemoglobin RNA represent approximately 98% and 70% of the RNA in whole blood, respectively, we tested standard commercial kits for removing these RNAs prior to RNAseq. 4 ml heparinized blood was treated and stimulated with 1 μg/ml LPS or was untreated and incubated for one hour at 37° C. Then, 250 μl of the unstimulated or stimulated blood sample was placed into 250 μl PAXgene fixative into replicate microtainer tubes. After RNA extraction, samples were either undepleted (left side of panel) or depleted (right side of panel) with the globin zero depletion kit and then quantitative PCR was performed to test for hemoglobin A2, 18S RNA, or TNF alpha mRNA expression. GlobinZero kits depleted both hemoglobin A2 and 18S ribosomal RNA (increased mean cycle time from 11 to 28 and 10 to 30, respectively) with relative preservation of TNFalpha mRNA. P values represent results of ordinary one-way ANOVA with Tukey's multiple comparisons test.

FIG. 8 provides RNASeq QC metrics of RNA with various quality scores prepared with Illumina TruSeq or Kapa Hyper Prep Kits. A. (Left Panel): Distribution of mapping, uniquely mapping, and duplicate reads. B. (Right Panel): Distribution of tags assigned to UTR (untranslated region), intergenic, intronic, and CDS (coding sequence) of whole blood RNA samples prepared with Illumina TruSeq or Kapa Hyper Prep Kits with various input RNA quality and quantity. The Illumina TruSeq library Prep demonstrated increased mapping to coding sequence and fewer intergenic reads and was ultimately used for downstream RNA sequencing experiments.

FIG. 9 provides clinical and transcriptional characteristics of RA flares in index patient. A. Index Patient disease activity over time. Disease activity (RAPID3 questionnaire, N=356), over the course of four years in index patient. Time points are colored according to disease activity category. B. Differential expression of genes in flare. Volcano plot of differential gene expression of flare (N=46) versus baseline (N=33), plotting statistical significance (−log10(FDR)) against fold change (log2(FC)) (gray points are non-significant genes, i.e., FDR>0.1, red indicates FDR<0.1 and log2 fold change >0, blue indicates FDR<0.1 and log2 fold change <0). Pathways enriched in significantly increased (C.) (Pathways increased in flare) or decreased genes (D.) (Pathways decreased in flare)in flare relative to baseline.

FIG. 10 provides transcriptional characteristics of immune activation prior to symptom onset in RA flares. A. Disease activity scores over time to flare (measured in days). Box represents disease activity from day −56 to +28 over time to flare. Vertical arrows (in A-D) represent start of flare. B. Hierarchical clustering of z scores of 2791 significantly differentially expressed genes over time to flare. Statistically significant clusters are labeled by color. AC2 and AC3 refer to clusters that changed antecedent to flare. C. Detailed representation of cluster 1, antecedent cluster 2 (AC2), and antecedent cluster 3 (AC3) genes from FIG. 3B over time to flare. D. Mean standardized cluster gene expression over time to flare. Light grey lines represent expression of individual genes in the cluster. Dashed horizontal line represents mean baseline gene expression (weeks −8 to −4). Dashed vertical line represents start of flare. E. Pathways enriched in clusters 1, AC2, and AC3.

FIG. 11. PRIME cells express AC3 genes. A. Synovial cell subtype marker genes in clusters identified in blood (FIG. 3A). Enrichment scores of 200 single cell RNAseq marker genes from 18 synovial subset cell types. Dashed line represents threshold for significance (FDR<0.05 or −log10 FDR>1.3). B. Mean standardized gene expression and 95% confidence intervals of genes common to synovial sublining fibroblasts (CD34+, DKK+ and HLA-DRA+ fibroblasts) and AC3 in blood over time to flare (dashed vertical line represents start of flare). Error bars represent confidence intervals. C. Venn diagram of AC3 genes that decrease during flare in 4 patients. D. Flow cytometry of blood samples from 19 RA patients and 18 healthy volunteers (HV). Percent PDPN+/CD45− cells of TOPRO-(live)/CD31− cells is presented. P value represents result of two sided t-test. E. Log2 fold change of AC3 genes expressed in PRIME cells (flow sorted CD45−/CD31−/PDPN+ cells) versus hematopoietic cells (flow sorted CD45+) and Log2 fold change of input cells (stained PBMC but not flow sorted) versus hematopoietic cells (flow sorted CD45+) as technical control for stress of flow sorting.

FIG. 12 depicts that differentially expressed flare genes are reproducibly altered in repeated flares. A. Index patient disease activity (RAPID3) over time. Top panel dots are colored by disease activity assignment. Bottom panel dots are colored according to clinical flare event number. B. Unsupervised hierarchical clustering of genes differentially expressed between baseline and flare. Top bar indicates samples colored according to disease activity assignment. Bottom bar indicates samples colored according to clinical flare event number. Data shows differentially expressed flare genes are represented by multiple clinical events.

FIG. 13 depicts that sorted PRIME cells express synovial fibroblast genes. Log2 fold change of various synovial single cell RNAseq marker genes in PRIME cells (flow sorted CD45−/CD31−/PDPN+ cells) versus hematopoietic cells (flow sorted CD45+) and Log2 fold change of Input cells (stained PBMC but not flow sorted) versus hematopoietic cells (flow sorted CD45+) as technical control for stress of flow sorting. These data show that single cell marker genes of fibroblasts (SC-F1, SC-F2, SC-F3, SC-F4) but not B cells (SC-B1-4), macrophages (SC-M1-4), or T cells (SC-T1-6) are enriched in sorted PRIME cells. Fibroblast genes (as marked) were the only set of synovial cell marker genes enriched in PRIME cells.

FIG. 14 depicts that sorted PRIME cells express classic synovial fibroblast genes. Volcano plot of Log10(−padj) vs Log2 fold change of PRIME cells (flow sorted CD45−/CD31−/PDPN+ cells) versus hematopoietic cells (flow sorted CD45+). Classic fibroblast genes are significantly increased in PRIME cells relative to hematopoietic cells.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The term “rheumatoid arthritis” or “RA” refers to a chronic disease, which is immune-mediated and inflammatory and is an autoimmune disorder, affecting the lining of joints that causes joint pain, stiffness, swelling and decreased movement of the joints and can eventually result in bone erosion and joint deformity. RA is a systemic autoimmune disease characterized by the simultaneous inflammation of the synovium of multiple joints.

An “RA flare” or “flare” refers to a surge in immune-mediated and/or inflammatory activity that is periodically experienced by a patient(s) with RA. During a flare, the level of fatigue and joint symptoms such as pain, swelling, and stiffness temporarily increase. Flares are periods of increased disease activity during which people's arthritis symptoms, which typically include joint pain, swelling, and stiffness, are more severe. An RA flare can involve an exacerbation of any symptom of the disease, but most commonly includes intense stiffness in the joints. People with RA report these common symptoms of flares: increased stiffness in joints, pain throughout the entire body, increased difficulty doing everyday tasks, swelling, such as causing shoes not to fit, intense fatigue, flu-like symptoms.

As used herein, “RNA” is defined as at least two ribonucleotides covalently linked together. The RNA may be any type of RNA. Examples include mRNA, tRNA, rRNA, shRNA, circRNA, scaRNA, scRNA, snRNA, siRNA or Piwi-interacting RNA, or a pri-miRNA, pre-miRNA, miRNA, snoRNA, long ncRNAs, anti-miRNA, precursors and any variants thereof. Further examples of RNA include RNA of a virus, or RNA sequences derived from a virus genome. Even further examples include RNA of a bacteria. RNA may be single stranded or double stranded, or may contain portions of both double stranded and single stranded sequence. RNA may be synthesized as a single stranded molecule or expressed in a cell (in vitro or in vivo) using a synthetic gene. RNA may be obtained by chemical synthesis methods or by recombinant methods.

RNA may also encompass the complementary strand of a depicted single strand. Many variants of RNA may be used for the same purpose as a given RNA. Thus, RNA also encompasses substantially identical RNA and complements thereof. A single strand provides a probe that may hybridize to a target sequence under stringent hybridization conditions. Thus, RNA also encompasses a probe that hybridizes under stringent hybridization conditions.

As used herein, “pg” means picogram, “ng” means nanogram, “ug” or “μg” mean microgram, “mg” means milligram, “ul” or “μl” mean microliter, “ml” means milliliter, “l” means liter.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

A “protease” as defined herein is an enzyme that hydrolyses peptide bonds. Conventional proteases may be used. Proteinase K is an example. It is preferred that the specific activity of the protease be high to degrade proteins in what can be a protein-rich sample and thereby protect the RNA from ribonucleases. The specific activity as determined by the Chromozym assay of the protease in the mixture of biological sample and denaturing solution is for example at least about 0.1 U/ml, at least about 1 U/ml, at least about 2.5 U/ml, at least about 5 U/ml, or at least about 10 U/ml. In another embodiment, the specific activity of the protease in the mixture is between 0.1 and 1000 U/ml.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

In accordance with the invention a method, kit and system have been established along with a clinical and technical protocol for isolation of RNA via repeated home blood collection of small volume blood samples using self-performed finger stick sampling by patients with a disease. The system enabled and allowed for longitudinal RNA sequencing (RNAseq). In an exemplary set of studies, rheumatoid arthritis (RA) patients were assessed and their RNA evaluated over a series of time points and correlated with clinical and physical parameters regarding RA flares. Samples were obtained from numerous (over 300) time points from eight flares over four years in an index patient, and over 200 time points from flares in three additional patients. A sampling method and RNA stabilization and isolation protocol were developed providing high quality intact RNA. Asssessments established that the RNAseq data from small volume blood finger stick samples correlated with blood cell counts from venipuncture blood draws. Transcripts were identified that were differentially expressed antecedent to RA flares. Transcriptomics of the patients prior to RA flares revealed a unique cell type, PRIME cells, in RA blood, which are predicted to become activated by B cells in the weeks prior to RA flare, and then migrate out of the blood to the synovium.

The methods and systems provided and enabled longitudinal genomic analysis via whole transcriptome analysis and total RNA sequencing (RNAseq). The studies provided herein establish that the collection system and RNA stabilization and isolation methods permits RNA sampling with valuable and consistent results. Among various applications for the system and methods, RNA profiling and longitudinal RNAseq analysis using the system and methods can reveal dynamic changes leading to flares of chronic inflammatory disease, provide indicators of clinical parameters and susceptibilities in disease or infection, reveal mechanisms via RNA activation and/or alteration in the progression of disease or infection or the susceptibility thereto, etc.

The present invention general relates to methods for RNA isolation and RNA profiling and analysis of small volume samples, wherein the RNA is of sufficient quality and quantity for whole transcriptome analysis and transcriptomic profiling.

Transcriptomics is the study of the ‘transcriptome,’ initially termed to signify an entire set of transcripts, and now widely understood to mean the complete set of all the ribonucleic acid (RNA) molecules expressed in some given entity, such as a cell, tissue, or organism. Transcriptomics can encompass everything relating to RNAs, including their transcription and expression levels, functions, locations, trafficking, and degradation. It can also include the structures of transcripts and their parent genes with regard to start sites, 5′ and 3′ end sequences, splicing patterns, and posttranscriptional modifications and covers all types of transcripts, including messenger RNAs (mRNAs), microRNAs(miRNAs), and different types of long noncoding RNAs (lncRNAs).

Modern transcriptomics often uses high-throughput methods to analyze the expression of multiple transcripts in different physiological or pathological conditions and this is rapidly expanding our understanding of the relationships between the transcriptome and the phenotype across a wide range of living entities. Whole-transcriptome analysis with total RNA sequencing (RNA-Seq) detects coding plus multiple forms of noncoding RNA and a goal of total RNA sequencing is to accurately measure gene and transcript abundance, and identify known and novel features of the transcriptome.

It is important to recognize that different levels of RNA evaluation and analysis require alternative amounts of RNA in terms or yield or quantity and in terms of quality. For instance, gene expression profiling experiments that are looking for a quick snapshot of highly expressed genes may only require a relatively small amount or lower quality RNA, particularly in as much as the amount of RNA from a highly expressed gene is more significant comparatively (as compared to a lower expressed or comparatively rare or small RNA) in a sample. Evaluation of targeted gene expression or assessing for the presence or absence of one or more targeted RNA may only require a relatively small amount or lower quality RNA, particularly in as much specific RNA probes or primer based isolation procedures may be utilized in the analysis. Experiments looking for a more global view of gene expression, and some information on alternative splicing, typically require a more mid level of quality and quantity of RNA. This encompasses most or many published RNA-Seq experiments for mRNA/whole transcriptome sequencing.

In important contrast, studies or experiments looking for or requiring an in-depth and full view of the transcriptome, to evaluate, identify or assemble new transcripts, to accurately measure gene and transcript abundance, or to identify known and novel features of the transcriptome require the highest quality and quantity of RNA available from samples. Thus, methods wherein RNA is isolated—even if from small or smaller volume samples—but is not quantitatively and qualitatively of the highest level, while suitable for some RNA analysis and study, will not be suitable for accurate and complete transcriptome profiling or longitudinal RNA profiling. There is an insufficient amount of all RNAs isolated to provide accurate and complete RNA information.

Total RNA-Seq analyzes both coding and multiple forms of noncoding RNA for a comprehensive view of the transcriptome and accurate and full results necessitate high quality RNA which is sufficient in quantity and yield to provide accurate, full length and comprehensive RNA sequences representing the full transcriptome. This then captures both known and novel features, allows researchers to identify biomarkers across the broadest range of transcripts, enables a more comprehensive understanding of phenotypes of interest and allows profiling of the whole transcriptome across a wide dynamic range.

The methods provided herein and in accordance with the invention provide RNA which is of sufficient quality and quantity for whole transcriptome analysis and transcriptomic profiling through RNA sequencing. Known and available methods for RNA isolation, if applied in a manner designed for larger volume samples, such as a standard venipuncture blood sample, or a sample of 2-3 mls of blood for example, do not result in RNA of suitable quality and quantity for whole transcriptome analysis and transcriptomic profiling through RNA sequencing when applied to small volume samples, particularly for example small blood samples from a fingerstick, or samples in the volume range of 100-300 μl blood.

In some embodiments, the sample may be a small volume blood sample, a sputum or saliva sample, or a nasal, nasopharyngeal or oropharyngeal swab, wash or aspirate. In embodiments, the small volume sample is a blood sample and is collected via fingerstick or heelprick. In an embodiment, the small volume sample is a blood sample and is collected via fingerstick. In embodiments, the fingerstick sample or heelprick sample may comprise blood droplets directly from a fingerstick or heelprick or a capillary tube may be utilized.

In some embodiments, the sample volume is less than 500 μl, less than 300 μl, less than 250 μl, about 200-300 μl, less than 200 μl, about 100-300 μl, about 150-300 μl, about 100-250 μl, about 50-300 μl, about 50-200 μl, about 50-150 μl. In an embodiment, the sample volume is about 100-300 μl. In some embodiments, the volume is less than 100 μl, less than 50 μl, about 10-50 μl, about 10-20 μl, about 10 μl, as small as 10 μl or less. In an embodiment, the sample volume is about 50-300 μl. In some embodiments, the sample volume is on the order of a blood droplet volume, or one or a few blood droplet volumes. In some embodiments, the blood or sample volume is that of a capillary tube volume, or less than a blood droplet volume. Capillary tube sample volumes may be on the order of 60-100 μl, 100-200 μl, 5-25 μl, 10-50 μl, less than 10 μl-5 μl. Capillary tubes on the order of these volumes are readily available commercially, such as from Sigma-Aldrich. The volume of choice or preference may be therein selected or as preferred.

A small volume sample may be from a patient or individual having a disease or infection or at risk for or suspected of disease or infection. In some embodiments, the patient or individual obtains or collects the small volume sample. In some embodiments, the patient or individual is assisted by a non-medical person in collection of the sample. In an embodiment, the sample is collected from a patient or individual by a non-medical person, such as a spouse, parent, friend, guardian, etc that is not medically trained or involved in any medical profession.

In accordance with the method, small volume sample(s) is collected and combined with an RNA stabilization solution. In some embodiments, the RNA stabilization solution is capable of lysing the cells in the sample and of stabilizing RNA contained in the cells or cell lysate of the sample. In some embodiments, the RNA stabilization solution is capable of lysing the cells in the sample and of stabilizing RNA contained in the cells or cell lysate of the sample in a single step. In embodiments, the sample and RNA stabilization solution are mixed, vortexed or shaken when combined. In some embodiments, the sample may be stored or left at room temperature for up to a few or several hours prior to refrigeration. In some embodiments, the sample is then stored in refrigerated conditions, such as at about 40° F. or about 4° C. for a brief time. In some embodiments, the sample is then stored in refrigerated conditions, such as at about 40° F. or about 4° C. for a brief time, up to a day or a few or several days. In some embodiments, the sample may be stored or left at room temperature for up to a few or several hours, up to 2 hours, up to 3 hours, up to 3 or 4 hours, prior to refrigeration. In some embodiments, the sample is then stored in refrigerated conditions, such as at about 40° F. or about 4° C. for a brief time, up to a day or a few or several days. In some embodiments, the sample is stored in a freezer or in frozen temperature conditions, such as at about 30 or 32° F. or about 0° C., either after collection, after brief (2-4 hour) storage at room temperature, or after brief (1-2 day) refrigerated storage.

In some embodiments, the volume of RNA stabilization solution is less than lml, about 500 μl or less, about 300 μl or less, about 200-300 μl, or about 250 μl. In an embodiment, the volume of RNA stabilization solution is about 300 μl or less, about 200-300 μl, or about 250 μl. In some embodiments, including wherein the sample volume is very low, such as on the order of less than 50 μl, or 10-50 μl, or about 10 μl, or less than 10 μl, the volume of RNA stabilization solution is appropriately low, such as on the order of less than 100 μl, less than 50 μl, less than 25 μl, as small as 10 μl or less

The RNA stabilization solution may be guanidinium based. The RNA stabilization solution may be a PAXgene based solution, a Tempus RNA based solution, a Trizol solution, a QIAzol-based solution, a Dxterity based solution system. Suitable guanidinium based solutions, such as guanidinium thiocyanate solutions are known. Guanidinium based solutions and methods have been previously described (for example Chomczynski P & Sacchi N. (1987) Anal. Biochem. 162: 156-159). Some solutions are or may be preferred and more advantageous or more suitable in the methods so as to generate RNA of sufficient quality and quantity for RNAseq and transcriptomic analysis or longitudinal analysis as provided herein.

The sample may collected into a tube or wherein the tube or receptacle for receiving the small volume sample and containing RNA stabilization solution has a total volume capacity of 1.5 ml or less, 1.2 ml or less, or lml or less, or 500 μl or less. In an embodiment, the sample is collected into a tube wherein the tube or receptacle for receiving the small volume sample and containing RNA stabilization solution has a total volume capacity of 1.5 ml or less, such as a microtainer tube. In some embodiments, the tube or receptacle for receiving the small volume sample and containing RNA stabilization solution is a tube which is suitable for small volumes, including very small volumes, such as a capillary tube. In an embodiment, the tube or receptacle is a capillary tube, which is suitable for small volumes, such as less than 100 μl, or even for very small volumes, such as less than 50 μl, less than 25 μl. Suitable sized tubes or containers are known and available in the art.

The invention provides a method for RNA profiling and analysis of small volume samples from a patient or individual comprising:
(a) obtaining one or more small volume sample self-collected by the patient or individual or by a non-medical person, wherein the sample is collected in or otherwise combined with an RNA stabilization solution whereby cells in the sample are lysed and RNA is stabilized; and
(b) isolating RNA using a process adapted for small volume samples wherein the amount of any and all solutions or buffers utilized are reduced and adjusted for small volume samples;
wherein the RNA is of sufficient quality and quantity for whole transcriptome analysis and transcriptomic profiling through RNA sequencing (RNAseq).

The RNA may be isolated using a process comprising:
(a) contacting the sample with a protease to form a protease treated small volume sample;
(b) contacting the protease treated sample with an ethanol or salt solution forming a precipitate containing the RNA, wherein the precipitate containing the RNA is then resuspended in a buffer or solution, or contacting the protease treated sample with an organic extraction solution, forming a solution having an aqueous phase containing the RNA and an organic phase;
(c) contacting the resuspended precipitate containing the RNA or the aqueous phase containing the RNA with DNAse to form a DNAse-treated resuspended precipitate or DNAse-treated aqueous phase;
(d) binding the RNA to a silica based solid phase or column by contacting the resuspended precipitate or aqueous phase with said silica based solid phase; and
(e) eluting the RNA from the silica based solid phase comprising contacting the silica based solid phase with a solution or buffer to provide isolated RNA;
wherein all buffer and solution volumes are reduced and adjusted for small volume samples.

In embodiments of the method, the RNA is isolated using a process comprising:
(a) contacting the sample with an RNA stabilization solution, wherein the solution has capability to lyse cells and inactivate adventitious agents;
(b) contacting the RNA stabilization solution treated sample with an ethanol or salt solution forming a precipitate containing the RNA, wherein the precipitate containing the RNA is then resuspended in a buffer or solution, or contacting the RNA stabilization solution treated sample with an organic extraction solution, forming a solution having an aqueous phase containing the RNA and an organic phase;
(c) contacting the resuspended precipitate containing the RNA or the aqueous phase containing the RNA with DNAse to form a DNAse-treated resuspended precipitate or DNAse-treated aqueous phase;
(d) binding the RNA to a silica based solid phase or column by contacting the resuspended precipitate or aqueous phase with said silica based solid phase; and
(e) eluting the RNA from the silica based solid phase comprising contacting the silica based solid phase with a solution or buffer to provide isolated RNA;
wherein all buffer and solution volumes are reduced and adjusted for small volume samples.

In embodiments of the method, the RNA is isolated using a process comprising:
(a) contacting the sample with an RNA stabilization solution, wherein the solution has capability to lyse cells and inactivate adventitious agents;
(b) optionally further contacting the sample with a salt, a reducing agent, and/or a detergent;
(c) contacting the solution contacted sample of (a) or (b) with silica, silica based solid phase or carboxylated magnetic beads which bind RNA and seves to purify the RNA from other components in the sample; and
(d) eluting the RNA from the silica or silica based solid phase or the magnetic beads comprising contacting the silica, silica based solid phase or magnetic beads with a solution or buffer to provide isolated RNA;
wherein all buffer and solution volumes are reduced and adjusted for small volume samples.

In embodiments, all buffer and solution volumes are reduced to about 20-30%, 20-28%, about 25% of the volumes for standard venipuncture blood, which is on the order of a sample volume of 2.5 mls. Thus, while the sample volume is about 1/10^{th} or 10% of the standard blood volume for commercial kits and methods, the buffers and solutions are reduced to about 20-30% or about 25%.

In commercial RNA isolation kits, such as the PAXgene Blood RNA kit the blood collection tube contains RNA stabilization solution appropriate for about 2.5 ml of sample volume. The PAXgene Blood RNA tube contains 6.9 ml of RNA stabilization solution, applicable for about 2.5 mls of blood. For the PAXgene Blood RNA tube, the relative ratio of sample volume to RNA stabilization buffer is about 0.36, or the stabilization solution volume is about 2.5-3 fold or about 2.76 fold the sample volume. In the present method, about 500 μl or less, about 300 μl or less, about 200-300 μl, or about 250 μl of RNA stabilization solution is present or provided for collection of the small volume sample. In the present method, about 500 μl or less, about 300 μl or less, about 200-300 μl, or about 250 μl of RNA stabilization solution is present or provided for collection of the small volume sample, wherein the sample volume is less than 500 μl, less than 300 μl, less than 250 μl, about 200-300 μl, about 250 μl, less than 200 μl, about 100-300 μl, about 150-300 μl, about 100-250 μl, about 50-300 μl, about 50-200 μl, about 50-150 μl. The range of sample volume to RNA stabilization buffer is on the order of about 5 fold to about 2 fold, about 5 fold to about 1 fold, about 3 fold to about 2 fold the sample volume. While the PAXgene kit blood collection tube contains 6.9 mls of RNA stabilization solution, in the instant methods the sample is combined with about about 250 μl or 0.25 mls which is a relative volume of 3-4%.

In commercial RNA isolation kits, such as the PAXgene Blood RNA kit, buffer volume for protease treatment is about 340 μl comprising 300 μl of buffer and 40 μl of protease. In the present method, buffer volume for protease treatment is about 74-75W comprising 65 μl of buffer and about 9 μl of protease. The relative volume percentage of the protease buffer and protease in the present method is about 20-22% or about 22%.

In some embodiments, between steps (b) and (c), the resuspended precipitate containing the RNA or the aqueous phase containing the RNA is contacted with a solution or column to remove residual sample cell debris and/or to homogenize the sample cell lysate.

The sample may be a small volume blood sample, a sputum or saliva sample, or a nasal, nasopharyngeal or oropharyngeal swab, wash or aspirate. In some embodiments, the sample is a small volume blood sample. In an embodiment, the small volume sample is a blood sample and is collected via fingerstick. In embodiments, the fingerstick sample may comprise blood droplets directly from a fingerstick or a capillary tube may be utilized.

In some embodiments of the method, the sample volume is less than 500 μl, less than 300 μl, less than 250 μl, about 200-300 μl, less than 200 μl, about 100-300 μl, about 150-300 μl, about 100-250 μl, about 50-300 μl. In an embodiment, the sample volume is about 100-300 μl. In some embodiments, the sample volume is less than 100 μl, less than 50 μl, less than 25 μl, 10 μl or less.

In some embodiments of the method, buffer and solution volumes are reduced to 20-40% or 20-30% or about 25% of those utilized for isolation of RNA from a standard venipuncture blood sample, such as a 2.5 ml or about 2.5 ml sample.

In some embodiments, the RNA stabilization solution is a guanidinium thiocyanate based or containing solution.

In some embodiments, any buffers or solutions are made or generated with RNAse free water or buffers.

In embodiments of the method, any suitable and efficacious protease is utilized. Suitable proteases are known and available in the art. In embodiment, the protease is proteinase K. In some embodiments, the sample is contacted and treated with a protease at a temperature above room temperature. In embodiments, the sample and protease are heated for protease treatment. In an embodiment, the sample and protease are heated to 50-60° C. or incubated at a temperature of 50-60° C. In an embodiment, the sample and protease are heated to or incubated at 55° C.

The purification/isolation method may be adapted for a may utilize a fully manual purification. In embodiments of manual purification centrifugation or a vacuum manifold, or a combination thereof, may be utilized, for example in order to pass solutions through columns. The purification/isolation method may be adapted for or may utilize Semi-automated purification. In embodiments of semi-automated purification, the lysis step and the precipitation or organic extraction step are carried out manually, while column purification is performed in an automated fashion, such as using an automated liquid handling system. Application of the isolation methods to fully automated purification is contemplated and an embodiment hereof, where all steps are performed using a fully automated system such as a fully equipped liquid handling system or a fully automated extraction system. Such fully automated systems are known and available in the art. In some embodiments, the fully automated systems are modified to adjust volumes, reagents, materials for small volume sample handling.

In embodiments of the method, commercial kits or RNA purification systems are modified. In embodiments, the PAXgene Blood RNA kit and process is modified for suitability and capability to provide for RNA isolation and RNA profiling and analysis of small volume samples, wherein the RNA is of sufficient quality and quantity for whole transcriptome analysis and transcriptomic profiling. In embodiments, the Tempus Blood RNA system and process is modified for suitability and capability to provide for RNA isolation and RNA profiling and analysis of small volume samples, wherein the RNA is of sufficient quality and quantity for whole transcriptome analysis and transcriptomic profiling.

The PAXgene protocol for Manual Purification of Total RNA from Human Whole Blood Collected into PAXgene Blood RNA Tubes is as follows (2015 Handbook):

Procedure
1. Centrifuge the PAXgene Blood RNA Tube for 10 minutes at 3000-5000×g using a swing-out rotor.
2. Remove the supernatant by decanting or pipetting. Add 4 ml RNase-free water to the pellet, and close the tube using a fresh secondary BD Hemogard closure (supplied with the kit).
3. Vortex until the pellet is visibly dissolved, and centrifuge for 10 minutes at 3000-5000×g using a swing-out rotor. Remove and discard the entire supernatant Small debris remaining in the supernatant after vortexing but before centrifugation will not affect the procedure.
4. Add 350 μl Buffer BR1, and vortex until the pellet is visibly dissolved.
5. Pipet the sample into a 1.5 ml microcentrifuge tube. Add 300 μl Buffer BR2 and 40 μl proteinase K. Mix by vortexing for 5 seconds, and incubate for 10 minutes at 55° C. using a shaker—incubator at 400-1400 rpm. After incubation, set the temperature of the shaker-incubator to 65° C. (for step 20).
6. Pipet the lysate directly into a PAXgene Shredder spin column (lilac) placed in a 2 ml processing tube, and centrifuge for 3 minutes at maximum speed (but not to exceed 20,000×g).
7. Carefully transfer the entire supernatant of the flow-through fraction to a fresh 1.5 ml microcentrifuge tube without disturbing the pellet in the processing tube.
8. Add 350 μl ethanol (96-100%, purity grade p.a.). Mix by vortexing, and centrifuge briefly (1-2 seconds at 500-1000×g) to remove drops from the inside of the tube lid.
9. Pipet 700 μl sample into the PAXgene RNA spin column (red) placed in a 2 ml processing tube, and centrifuge for 1 minutes at 8000-20,000×g. Place the spin column in a new 2 ml processing tube, and discard the old processing tube containing flow-through.
10. Pipet the remaining sample into the PAXgene RNA spin column, and centrifuge for 1 minutes at 8000-20,000×g. Place the spin column in a new 2 ml processing tube, and discard the old processing tube containing flow-through.
11. Pipet 350 μl Buffer BR3 into the PAXgene RNA spin column. Centrifuge for 1 minute at 8000-20,000×g. Place the spin column in a new 2 ml processing tube, and discard the old processing tube containing flow-through.
12. Add 10 μl DNase I stock solution to 70 μl Buffer RDD in a 1.5 ml microcentrifuge tube. Mix by gently flicking the tube, and centrifuge briefly to collect residual liquid from the sides of the tube.
13. Pipet the DNase I incubation mix (80 μl) directly onto the PAXgene RNA spin column membrane, and place on the benchtop (20-30° C.) for 15 minutes.
14. Pipet 350 μl Buffer BR3 into the PAXgene RNA spin column, and centrifuge for 1 minute at 8000-20,000×g. Place the spin column in a new 2 ml processing tube, and discard the old processing tube containing flow- through.
15. Pipet 500 μl Buffer BR4 into the PAXgene RNA spin column, and centrifuge for 1 minute at 8000-20,000×g. Place the spin column in a new 2 ml processing tube, and discard the old processing tube containing flow-through.
16. Add another 500 μl Buffer BR4 to the PAXgene RNA spin column. Centrifuge for 3 minutes at 8000-20,000×g.
17. Discard the processing tube containing the flow-through, and place the PAXgene RNA spin column in a new 2 ml processing tube. Centrifuge for 1 minute at 8000-20,000×g.
18. Discard the processing tube containing the flow-through. Place the PAXgene RNA spin column in a 1.5 ml microcentrifuge tube, and pipet 40 μl Buffer BR5 directly onto the PAXgene RNA spin column membrane. Centrifuge for 1 minute at 8000-20,000×g to elute the RNA.
19. Repeat the elution step (step 18) as described, using 40 μl Buffer BR5 and the same microcentrifuge tube.
20. Incubate the eluate for 5 minutes at 65° C. in the shaker—incubator (from step 5) without shaking. After incubation, chill immediately on ice.
21. If the RNA samples will not be used immediately, store at −20° C. or −70° C.

The PAXgene Blood RNA system and method is specifically and particularly designed and applicable for blood sample volumes of about 2.5 mls, which is on the order of 10 fold larger volumes than the methods herein are processing. The PAXgene Blood RNA system and handbook provide a Troubleshooting Guide for issues with the system and notes that this troubleshooting guide may be helpful in solving any problems that may arise. With regard to Low RNA yield, the Troubleshooting Guide indicates: “Less than 2.5 ml blood collected in PAXgene Blood RNA Tube. Ensure that 2.5 ml blood is collected in the PAXgene Blood RNA Tube” (see PAXgene Blood RNA Tube Product Circular). The PAXgene blood RNA system is admittedly not designed for or successfully applicable to small volume samples.

A comparison of the PAXgene Blood RNA system procedure and RNA isolation method with the methods provided herein including in Example 1, will demonstrate that the volumes utilized, particularly including in each of steps are significantly reduced and are approximately 20- % of the volume indicated. Sample volumes of approximately 1/10^{th} or 10% volume size of those recommended and best for the Paxgene system can be processed with approximately 25% volume size of the buffers and solutions to successfully provide RNA of sufficient quality and quantity for whole transcriptome analysis and transcriptomic profiling through RNA sequencing.

In accordance with an embodiment of the method, in comparison with commercial RNA isolation kit volumes, such as particularly the PAXgene Blood RNA kit method and procedure outlines above, the volume of buffer (water) in step 2. is 1 ml, which is 25% of the 4 ml in the kit method. In accordance with an embodiment of the method, the volume of buffer in step 4. is 75 μl, which is 21.4% of the 350 μl in the kit method. In accordance with an embodiment of the method, the volume of buffer in step 5. is 65 μl buffer and 9 μl proteinase K, which is 21.7% of the 300 μl and 22.5% of the 40 μl in the kit method. In accordance with an embodiment of the method, the volume of ethanol solution in step 8. is 75μ1, which is 21.4% of the 350 μl in the kit method. In accordance with an embodiment of the method, the volume of buffer in step 11. is 100 μl, which is 28.5% of the 350 μl in the kit method. In accordance with an embodiment of the method, the volume of buffer in step 14. is 100 μl, which is 28.5% of the 350 μl in the kit method. Volume adjustments of buffers and solutions in the present method range from about 21% to about 29% or overall about 25%.

Robison and colleagues previously reported a general assessment of transcript profiling from fingerstick blood samples (Robison EH et al (2009) BMC Genomics 10:617, doi:10.1186/1471-2164-10-617). Only RNA quality and broad correlations of gene expression data using genechip analysis comparing fingerstick samples with whole blood samples were reported. Robison followed the PAXgene Blood RNA kit (product #762164) protocol for RNA isolation and purification, with the exception of one modification, wherein after the first spin, the pellet was washed with 1 mL RNase free water instead of 4 mL due to its small volume. Robison reported that they tested a scaled down version of the PAXgene protocol, but found that using the standard volumes of buffers and washes had no effect on the yields and were preferred as easier to employ. This is in sharp contrast to the studies and results reported and provided herein.

The methods herein may further comprise sequencing the RNA. RNA may be sequenced using any suitable or recognized method, steps, system(s) or kit(s), including manual, semi-automated or automated method(s), system(s) or kits. In some embodiments, kits such as Illumina TruSeq or Kapa Hyper Prep Kits are utilized.

As part of or commensurate with the methods herein, the isolated RNA may converted to cDNA. Methods for generating cDNA from RNA are well known and available to one skilled in the art. Any applicable and effective method should be suitable. The isolated RNA may be converted to cDNA for probing or specific primer applications, such as to assess expression or for sequencing of specific RNAs or gene products. The isolated RNA may be converted to cDNA for cloning purposes, to be inserted or prepared in a vector, for introducing into or preparing a library therefrom.

As part of or commensurate with the methods herein, the isolated RNA may amplified. In some embodiments, theRNA may be converted to cDNA and then amplified. Suitable methods and systems for amplification are known and available. For instance, methods, kits and systems for PCR amflication, including RT-PCR, wherein RNA is first reverse transcribed to cDNA and then amplifies are well known and available. Amplification methods and approaches may be useful particularly in the instances of small volume samples and/or where small amounts of RNA are being isolated. Another amplification approach, which is also useful for small volume or small quantity RNA samples, is loop-mediated isothermal amplification (LAMP). Combining LAMP with a reverse transcription step allows detection and evaluation of RNA. LAMP is carried out at a constant temperature (60-65° C.) and thus does not require a thermal cycler. LAMP mathods may utilize Bst (Bacillus stearothermophilus) DNA polymerase.

Abundant RNA species or RNA species not of interest may be removed prior to RNA sequencing. For example, globin mRNA, ribosomal RNA(s) and/or species specific RNAs may removed prior to sequencing. In some instances, globin RNA and ribosomal RNAs are both removed. This serves to eliminate highly prevalent RNAs or known RNAs which are not of interest from the isolated RNAs. Eliminating highly prevalent or irrelevant globin RNA or rRNAs may facilitate analysis of RNAs which are of interest or which are less prevalent and present in smaller amounts. Methods, systems and kits for removal of globin RNA and/or ribosomal RNA are know and available to one skilled in the art. In some embodiments, systems or kits such as BlobinZero (Illumina), Ribo-Zero Gold, TruSeq Stranded total RNA library prep, Ribo-Zero Globin, GLOBINclear kit (THermo Fisher Scientific), QIAseqFastSelect RNA removal kit (Qiagen) may be utilized. In some embodiments, species specific probes may be utilize to select out certain RNAs.

In embodiments or the method, the patient or individual has a disease or infection or is at risk of or suspected of disease or infection. The disease may be an acute or chronic disease. The disease may be a relapsing and/or remitting disease. The infection may be a bacterial or viral infection. The infection may be with a known or unknown virus or bacteria. A viral infection or virus may be an influenza virus, a coronavirus, an unidentified virus, an RNA virus. A bacteria may be a gram-positive bacteria. A bacteria may be a Streptococcus or Staphylococcus bacteria. A disease may be an inflammatory disease, an immune disease, an auto-immune disease, cancer.

In some embodiments, the method is for longitudinal screening by RNA profiling and analysis of small volume samples from one or more patient or individual, wherein the patient or individual has a disease or infection or is at risk of or suspected of disease or infection. In embodiments, small volume samples are collected in series or in regular or designated increments of hours, days, weeks or months. Small volume samples of a small volume blood sample, a sputum or saliva sample, or a nasal, nasopharyngeal or oropharyngeal swab, wash or aspirate may be collected. A combination of sample types or varying sample types may be collected. In embodiments, small volume blood samples are collected via fingerstick in series or in regular or designated increments of hours, days, weeks or months.

Samples may be collected in several hour increments, twice a day, three or four times a day, every 4-6 hours, daily, every morning, every evening, every morning and evening, once a week, one a month, every two months, every four months, every six months, several times a year. Samples may be collected to evaluate the effects of a drug or agent, for example prior to and/or following administration of a drug or agent. In some embodiments, small volume samples may be collected or additionally collected at outset of symptom(s), such as one or more symptom or recognized parameter indicative of or associated with a disease or infection. Samples may be collected prior to and after or upon the recognition or development of one or more symptom or disease or infection parameter. Samples may be collected upon the development of a fever, cough, pain or discomfort, rash, etc.

Systems and kits for use and application of the methods are provided. A system or kit is provided for RNA profiling and analysis of small volume samples from a patient or individual comprising:
(a) a means for self-collection of a small volume sample by the patient or individual or by a non-medical person, comprising a lancet, swab or receptable for a wash, spit or aspirate;
(b) a tube or receptacle for receiving the small volume sample on collection and containing a volume of RNA stabilization solution whereby cells in the sample are lysed and RNA is stabilized; and
(c) one or more appropriate label(s) for designating the name or identity of the patient or individual, date of sample collection and time of sample collection.

In embodiments, the system or kit may further comprise an envelope or mailing container for shipment of the sample to a laboratory or facility for RNA isolation and analysis.

In an embodiment, with collection, the first drop of blood is removed, for example with a sterile gauze or cotton ball, so as to avoid tissue fluids that may produce inaccurate or less effective results. In an embodiment, the finger, heel etc, is cleansed with an alcohol or detergent solution, wipe or swab prior to collection, so as to remove any surface debris, loose cells or bacteria or dirt.

In some embodiments the lancet may be a small manual blade or may be a spring-loaded assembly or a self-contained disposable unit, such as wherein the blade is automatically retracted a holder after use. One such example is the Dynarex SensiLance pressure activated lancet.

In some embodiments, the system or kit may be for longitudinal RNA profiling and analysis of multiple small volume samples collected in series from a patient or individual over days, weeks or months comprising:
(a) a set of numerous means for self-collection of individual small volume samples by the patient or individual or by a non-medical person, each comprising a lancet, swab or receptable for a wash, spit or aspirate;
(b) a set of numerous tubes or receptacles each individually for receiving a small volume sample on collection and containing a volume of RNA stabilization solution whereby cells in the sample are lysed and RNA is stabilized;
(c) numerous appropriate label(s) for designating the name or identity of the patient or individual, date of sample collection and time of sample collection; and
(d) numerous envelopes or mailing containers for shipment of each sample or several samples to a laboratory or facility for RNA isolation and analysis.

In some embodiments of the system or kit, the volume of RNA stabilization solution is less than 1 ml, about 500 μl or less, about 300 μl or less, about 200-300 μl, or about 250 μl.

In some embodiments of the system or kit, the tube or receptacle for receiving the small volume sample and containing RNA stabilization solution has a total volume capacity of 1.5 ml or less, 1.2 ml or less, or lml or less.

In the specification, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Throughout this specification, quantities are defined by ranges, and by lower and upper boundaries of ranges. Each lower boundary can be combined with each upper boundary to define a range. The lower and upper boundaries should each be taken as a separate element.

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such non-limiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

### Isolation and Analysis of RNA From Rheumatoid Arthritis Patient Finger Stick Blood Samples: RNA-Based Longitudinal Genomics Identifies Markers of RA Flares

Rheumatoid arthritis (RA), like many inflammatory diseases, is characterized by episodes of quiescence and exacerbation (flares). The molecular events leading to flares are unknown. We established a method, kit and system along with a clinical and technical protocol for isolation of RNA via repeated home blood collection of small volume blood samples using self-performed finger stick sampling by RA patients. The system enabled and allowed for longitudinal RNA sequencing (RNAseq). Samples were obtained from 364 time points from eight flares over four years in our index patient, and 235 time points from flares in three additional patients. We developed a sampling method and RNA stabilization and isolation protocol providing high quality intact RNA. Asssessments established that the RNAseq data from small volume blood finger stick samples correlated with blood cell counts from venipuncture blood draws. We identified transcripts that were differentially expressed antecedent to flares and compared these to synovial single cell RNAseq (scRNAseq). Flow cytometry and sorted blood cell RNAseq in additional RA patients were used to validate the findings.

Consistent changes were observed in blood transcriptional profiles one to two weeks antecedent to RA flare. B cell activation was followed by expansion of a previously unexplored circulating CD45−/CD31−/PDPN+, PRe-Inflammatory MEsenchymal (“PRIME”) cell in RA patient blood, which shared features of inflammatory synovial fibroblasts. Circulating PRIME cells decreased during flares from all four patients, and flow cytometry and sorted cell RNAseq confirmed the presence of PRIME cells in 19 additional RA patients.

Longitudinal genomic analysis of RA flares reveals PRIME cells in RA blood, and suggests a model in which they become activated by B cells in the weeks prior to RA flare, and then migrate out of the blood to the synovium. These studies established that the collection system and RNA stabilization and isolation methods permits RNA sampling with valuable and consistent results. Among various applications for the system and methods, RNA profiling and longitudinal RNAseq analysis using the system and methods can reveal dynamic changes leading to flares of chronic inflammatory disease.

Introduction

Rheumatoid arthritis (RA) symptoms are highly dynamic, with stable periods interrupted by unpredictable flares of disease activity. Such waxing/waning clinical courses are characteristic of many autoimmune diseases, including multiple sclerosis (1), systemic lupus erythematosus (2), and inflammatory bowel disease (3,4), underscoring a need to develop approaches to understand what triggers transitions from quiescence to flare in autoimmune disease.

This study explores disease pathophysiology with a longitudinal, prospective analysis of blood transcriptional profiles in individual RA patients over time utilizing small volume blood sampling via patient at home finger stick collection. Previous microarray studies of RA blood samples from relatively sparse time series data have identified few significant gene changes associated with disease activity (5-8). Here we provide the first RA study to look for molecular changes in blood that anticipate clinical flares. To do so we developed and optimized methods by which RA patients themselves could collect high quality finger stick blood samples for RNA sequencing (RNAseq), facilitating weekly blood sampling for months to years.

We analyzed patient reports of clinical disease activity and RNAseq data from four patients across multiple clinical flares. In our most deeply studied index case, we assessed 364 time points by RAPID3 from eight flares over four years, and analyzed 84 time points assessed by RNAseq. Collecting samples longitudinally enabled a search for transcriptional signatures that preceded clinical symptoms. Comparing these blood RNA profiles to synovial single cell RNAseq (scRNAseq) data (9) provided evidence that a biologically coherent set of transcripts are significantly increased in the blood prior to symptom onset, and a subset of these decrease as the patients begin to experience symptoms. These latter transcripts overlap with and likely demarcate cellular precursors to a novel subset of synovial sublining fibroblast cell types detected in inflamed RA synovium using scRNAseq. Analysis in 19 additional RA patients corroborated our findings. Our data suggests a model in which a previously unexplored circulating mesenchymal cell type, detectable in the weeks prior to RA flare, becomes activated by B cells and subsequently leaves the blood, traffics to synovium, and contributes to disease activity. These studies were facilitated by the ability to isolate and analyze high quality RNA which validly represented changes in vivo from patient self-collected small volume blood samples (finger stick).

Methods

Patient Data

All patients met American College of Rheumatology/European League Against Rheumatism 2010 (10,11) criteria for RA and were seropositive for cyclized citrullinated protein antibody (CCP). Disease activity was assessed from home each week, or up to 4 times daily during escalation of flares, using the routine assessment of patient index data 3 (RAPID3) questionnaire (12). Disease activity was also assessed at clinic visits, each month, and during flares, using both the RAPID3 and the disease activity score 28 (DAS28), which incorporates tenderness and swelling from 28 joints, erythrocyte sedimentation rate (ESR) and patient global assessment of disease activity. Complete blood counts (CBC) including white blood cells (WBC), neutrophils, monocytes, lymphocytes, and platelets were performed by the clinical lab at Memorial Sloan Kettering Cancer Center. We collected 43 clinic visits from the index patient, and 25, 14 and 12 clinic visits for the other three patients studied longitudinally. Nineteen additional seropositive RA patients and 18 age and sex matched non-RA patients, for whom peripheral blood mononuclear cells (PBMC) were available, were also studied for the presence of PRIME cells by FACS and RNAseq analysis.

RNA Preparation from Finger Stick Blood

Patients self-performed finger sticks at home to collect three drops of blood into a microtainer tube prefilled with fixative (RNA stabilization solution), and samples were mailed overnight each week. RNA was extracted using the PAXgene RNA kit and purified per manufacturer's protocols, except the volume of all washes and elutions was decreased to about 25% of the recommended volume by the manufacturer. RNA was assessed using the Agilent BioAnalyzer for quantity and quality. For library preparation, we used the GlobinZero kit (EpiCentre #GZG1224) and Illumina's Truseq mRNA Stranded Library kit, with 11-12 PCR cycles for 5-8 nM input and sequenced on HiSeq2500 with 150 base paired-end reads. Reads were aligned to Gencodev18 using STAR and quantified using featureCounts (v1.5.0-p2). Samples with at least four million paired-end reads were retained for analysis.

A detailed protocol is provided below:

### Procedure (SOP) Finger Stick Sample Processing

Should receive a box with patient questionnaire and finger stick sample

Place ice pack in cardboard box lined with chuck to thaw and dry

Make notes in Sample Log:
Date of sample acquisition (date of finger stick)- Date on questionnaire
Today's date
Study Week # (if it's not clear from previous week, calculate back from Week 0 on top of sample log page)

Finger Stick Sample
Three drops of blood are received in microtainer tube that was prefilled with 0.250 ml (250 μl) PAX-gene blood solution.
Write the date (of the finger stick) on a finger stick label
Transfer to −20° C. for 24 hours. Store upright in metal rack.
Transfer to −80° C. for long term storage in patient's sample box in −80° C. with the week # written on top of the tube.

(adapted from PAXgene RNA handbook version 2, June 2015)

Before starting

Set temperature of shaker incubator to 55° C.

Warm Buffer BR2 (Binding Buffer) to 37° C. if there are precipitates (Binding Buffer contains a guanidine salt (guanidine thiocyanate) which can form highly reactive compounds when combined with bleach)

Prepare Buffer BR4 (Wash Buffer) by adding 4 volumes of 100% ethanol to obtain working solution

Prepare DNAse I stock by dissolving solid DNAse I (1500 Kunitz units; Qiagen, cat #79254) in 550 ul of RNAse free water and mix by inversion (1500 Kuntz Units/0.55 ml). Do not vortex, DNAse is sensitive to physical denaturation.

1. Remove PAXgene Blood RNA microtainer tube from freezer and allow to warm to room temperature (+/−1 hr)

2. Place PAX blood in 2 ml microfuge tube and centrifuge at 5000×g at room temperature for 10 minutes

3. Remove and discard supernatant. Add 1 ml RNAse-free water (from PAXgene kit) to wash the pellet.

4. Vortex to resuspend the pellet, then centrifuge for 10 minutes at 5000×g in a centrifuge. Remove and discard the supernatant.

5. Thoroughly resuspend the pellet in 75 ul of Buffer BR1 (from PAXgene kit) (Resuspension Buffer) by vortexing

6. Add 65 ul Buffer BR2 (from PAXgene kit) and 9 ul Proteinase K solution (from PAXgene kit).

7. Mix by vortexing and incubate for 10 minutes at 55° C. in shaking heat block (800 rpm).

8. Pipet lysate to lavender top PAXgene shredder spin column (which removes clumps) and spin for 3 minutes at 18,000×g.

9. Transfer supernatant of flow through (about 150 ul) ** be careful with this step since pellet is gooey and easily disrupted** to a new 1.5 ml microcentrifuge tube.

10. Add 75 ul of 100% ethanol. Mix by vortexing and centrifuge at 1000×g for 2 seconds to remove drops from inside the tube of lid. Do not centrifuge for longer than this or nucleic acids may pellet and reduce the RNA yield.

11. Apply 225 ul of sample to red top PAXgene RNA spin column sitting in a 2 ml processing tube (from PAXgene kit). Centrifuge at 8000×g for 1 minute. Place the PAXgene column in a new 2 ml processing tube and discard the old processing tube containing the flow through.

12. Pipet 100 ul Buffer BR3 (Wash Buffer) to the PAXgene column and centrifuge at 8000×g for 1 minute. Place the PAXgene column in a new 2 ml processing tube and discard the old processing tube containing flow through.

13. Pipet 5 ul DNAase I stock solution into 35 ul of Buffer RDD. Mix by gently flicking the tube (do not vortex) and centrifuge briefly.

14. Pipet DNAse I incubation mix (40 ul) directly onto PAXgene column and place upright at room temperature for 15 minutes.

15. Pipet 100 ul Buffer BR3 to the PAXgene column and centrifuge at 8000×g for 1 minute. Place the PAXgene column in a new 2 ml processing tube and discard the old processing tube containing flow through.

16. Apply 200 ul Buffer BR4 (Wash Buffer) to the PAXgene column and centrifuge for 1 minute at 8000×g. Place the PAXgene column in a new 2 ml processing tube and discard the old processing tube containing flow through. Note that the Buffer BR4 is supplied as a concentrate. Ensure that the ethanol is added to Buffer BR4 prior to use.

17. Add another 200 ul Buffer BR4 to the PAXgene column. Centrifuge for 3 minutes at 18000×g (max speed) to dry the PAXgene column membrane.

18. To eliminate residual Buffer BR4, discard the tube containing the flow through, place the PAXgene column in a 2 ml processing tube and centrifuge for 1 minute at full speed.

19. Discard the tube containing the flow through and transfer the PAXgene column to a 1.5 ml elution tube (from PAXgene kit). Pipet 30 ul Buffer BR5 directly on to the PAXgene column membrane (without touching the membrane with the pipet tip) and centrifuge for 2 minutes at 13000×g.
20. Repeat the elution step as described, using the previously eluted 30 ul of RNA in Buffer BR5 (Elution Buffer). 2 ul for Pico Bioanalyzer and 26 ul for GlobinZero
21. Label and store sample at −80° C. until RNA analysis.

Data Analysis:

Differential Expression Analyses Across Patients

Samples were labeled “baseline” (stable RAPID3), “flare” (RAPID3 scores rose over two standard deviations above the baseline mean), or “steroid”. EdgeR (v3.24.3) (13) was used to analyze flare vs baseline differential gene expression. Permutation test (n=1×106) was used to test for the significance of overlap between genes decreased in flares in the index patient and patients 2, 3, and 4. GO enrichment (goana, from limma v3.38.3) (14) was used to identify enriched pathways in significantly differentially expressed genes in the index patient (FDR<0.1) and consistent in the direction of expression in both the index and replication patients (i.e., log fold change either both positive or both negative).

Time Series Analysis of Index Patient

We performed longitudinal data analysis on the index patient using ImpulseDE2 (v1.8.0) (15). Flare onset was defined clinically (as above) and samples from 8 weeks prior to flare up to 4 weeks after flare were analyzed (excluding any samples during which the patient was taking steroids, n=65 samples). The date of library preparation was included in the model for batch correction, and the genefilter (v1.64.0) package (16) was used to filter out lowly expressed genes. We hierarchically clustered mean expression of significantly differentially expressed genes by week to flare initiation (batch corrected logrpkm expression values were calculated using edgeR) and identified five coexpressed gene modules (Clusters 1-5). We analyzed these five modules for GO term enrichment (goana).

To compare differentially expressed gene modules over time, the mean expression level for each gene was calculated across flares per week, then normalized across weeks. ABIS (17) and CIBERSORTx (18) were used to deconvolute gene expression data. To aggregate a given cluster of genes or cell type with gene markers, the mean of standardized gene expression scores or deconvolved cell type scores, respectively, within each week were plotted. To identify synovial scRNAseq cluster specific marker gene signatures, we used a previously published dataset (18) to compare the cells from one scRNAseq cluster with cells from all the other scRNAseq clusters using the single-cell RNA-seq log2(CPM+1) matrix. We generated lists of the top 200 marker genes for each cluster using the criteria of 1) log2FC greater than 1, 2) auc greater than 0.6, and 3) percent of expressing cells greater than 0.4. We used Fisher's exact test to evaluate enrichment of synovial cell subtype marker genes in the 5 coexpressed gene modules.

Flow Cytometry and Sorting

Samples from PBMC were stained with antibodies to: CD31-APC, (WM59), Mouse IgG1-APC, (MOPC-21), PDPN-PerCP, (NZ1.3), Rat IgG2a, (eBR2a), CD45-PE, (HI30), Mouse IgG1-PE, (MOPC-21), TO-PRO®-3, and DAPI (4′,6-Diamidino-2-Phenylindole, Dihydrochloride). Cells were sorted on a BD FACSAria II for RNAseq. cDNA libraries were sequenced on MiSeq. DESeq2 (v1.24.0) (19) was used for differential expression analysis.

Statistics

R2 and Pearson correlation coefficients were calculated to assess the bivariate linear fit of disease activity measured by RAPID3 and DAS28 as well as CBC counts inferred from CIBERSORT cell counts and counts measured by clinical labs. Inferred CIBERSORTx lymphocyte counts were the sum of B cells naive+B cells memory+T cells CD8+T cells CD4 naive+T cells CD4 memory resting+T cells CD4 memory activated. One way ANOVA was used to test for significant differences among various clinical features according to disease activity state. Monocytes, Macrophages MO, Macrophages M1, and Macrophages M2 were summed to infer CIBERSORTx monocytes.

Results

Clinical Protocol Development

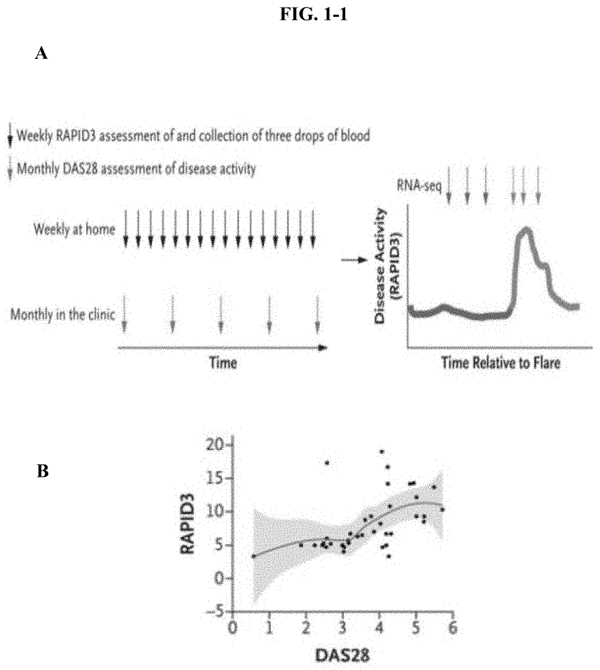

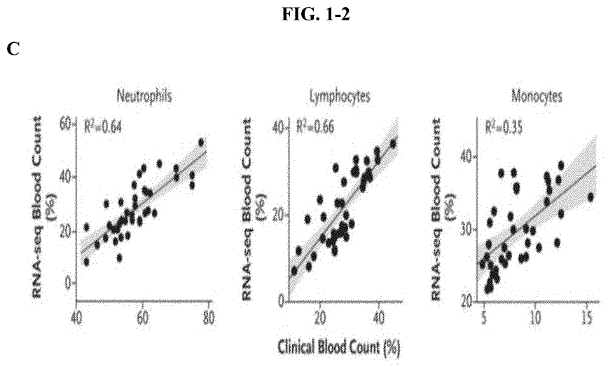

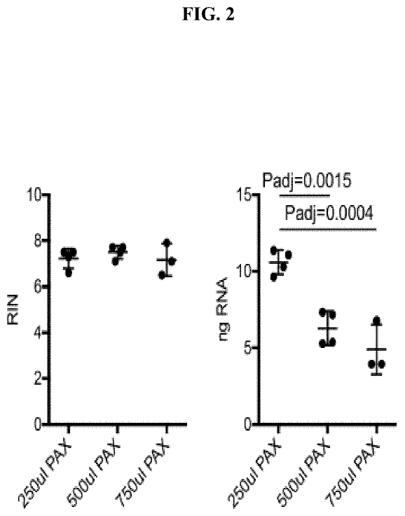

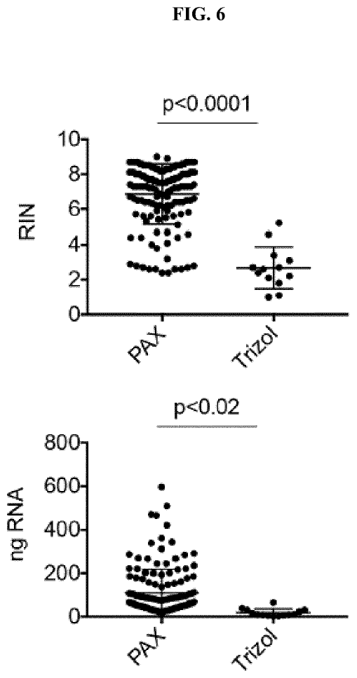

Four RA patients were followed for one to four years with weekly home collection of finger stick blood samples coupled with completion of RAPID3 and monthly clinic visits, where DAS28 were collected (FIG. 1A). Study patients also documented disease activity (RAPID3 questionnaires). We developed strategies for home blood collection that would allow high quality and quantity RNA for sequencing (FIGS. 2-8), which provided 15-50 ng RNA from finger stick blood samples and RNA integrity (RIN) scores (mean 6.9+/− standard deviation 1.7).

RNA was sequenced from a total of 189 finger stick blood samples from 4 patients, of which 162 (87%) passed quality control filtering.

We first assessed RNA quality and quantity by volume of fixative. 3 drops of blood were harvested with a 21 guage lancet and added to a microtainer tube prefilled with either 250 μl, 500 μl or 750 μl of PAX gene fixative. Samples were stored at room temperature for 3 days and then RNA was extracted using the PAX gene RNA kit and RIN scores and quantity of RNA was assessed using the Agilent 2100 Bioanalyzer picochip. RIN indicates the RNA integrity number which is an algorithm for assessing integrity values to RNA. The integrity of RNA is of significant importance for gene expression studies. RIN can and was traditionally evaluated using the 28S (˜5070 nucleotides) to 18S (˜1869 nucleotides) RNA ratio, which gives a ratio of about 2.7. A high 28S to 18S ratio is an indication that the purified RNA is intact and hasn't been degraded. RIN can easily be determined using Agilent 2100 Bioanalyzer measurements (Schroeder A et al (2006) BMC Mol Biol 7:3 (doi:10.1186/1471-2199-7-3). RNA samples should score RIN of >7 on a scale of 1 (highly degraded) to 10 (highest integrity). The results are depicted in FIG. 2. Acceptable RIN scores are seen with 250 μl, 500 μl or 750 μl of PAX-gene fixative (left panel of FIG. 2). Notably, the 250 μl fixative results in the highest ng RNA yield per sample. Using higher volumes of fixative, either 500 μl or 750 μl of fixative, the ng RNA yields were significantly reduced compared with 250 μl fixative (right panel of FIG. 2).

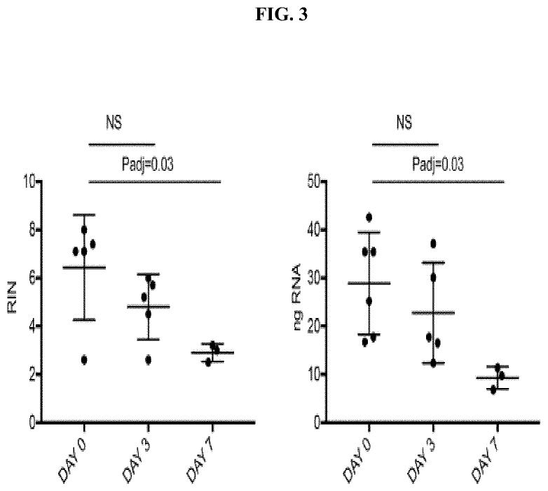

RNA integrity/quality and RNA quantity was evaluated from samples of 100 μl of blood in 250 μl PAX gene fixative with varying times of storage at room temperature (FIG. 3). 100 ul of whole blood was added to a microtainer tube prefilled with 250 μl PAX gene fixative and frozen after 2 hours, 3 days, or 7 days incubation at room temperature. RNA was extracted with scaled down washes and elutions using the protocol described above and RIN scores and quantity of RNA was assessed using the Agilent 2100 BioAnalyzer RNA picochip. RNA quality and quantity is reasonably retained with room temperature storage for up to 3 days.

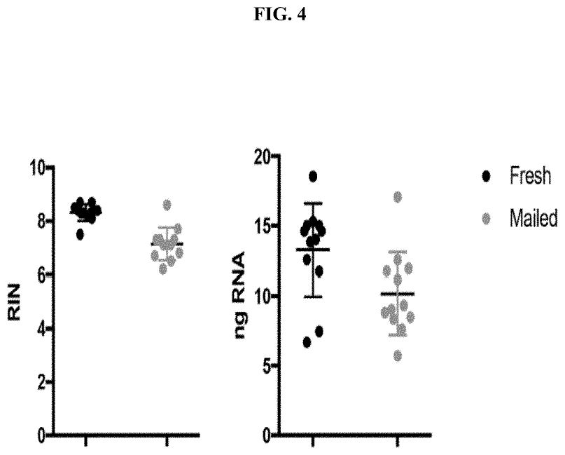

RNA quality and quantity were evaluated in fresh and mailed samples (FIG. 4). 100 μl of whole blood was added to a microtainer tube prefilled with 250 μl PAX gene fixative and frozen after 2-hour incubation at room temperature or mailed RNA was extracted as above described and RIN scores and quantity of RNA was assessed using the Agilent 2100 BioAnalyzer RNA picochip. RIN and quantity of RNA was well maintained with mailing of samples.

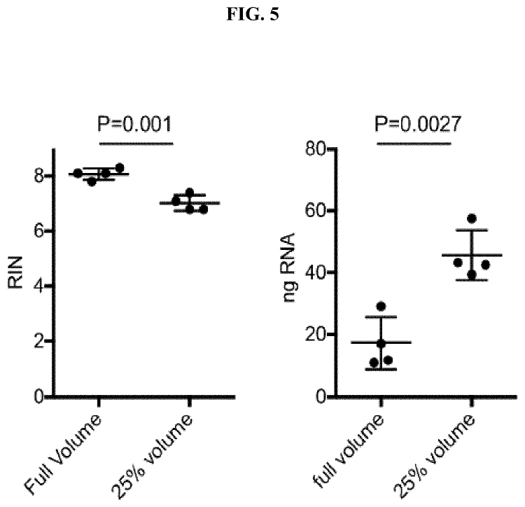

RNA quality and quantity were evaluated by volume of extraction and washes (FIG. 5). 3 drops of blood harvested with a 21 guage lancet were added to a microtainer tube prefilled with 250 μl of PAX gene fixative. Samples were stored at room temperature for 3 days and then RNA was extracted using the PAXgeneRNA kit according to manufacturer' s directions or with a scaled down version of the PAX protocol, using significantly reduced volumes (about 25% of the recommended volumes) for all washes and elutions. RIN scores and quantity of RNA was assessed using the Agilent 2100 BioAnalyzerRNA picochip. The RIN score was well maintained in the low volume protocol. Quantity of RNA isolated, however, was significantly improved with the low volume protocol. This demonstrates that a reduced volume protocol was necessary to isolate a reasonable quantity of RNA from small blood volume samples, such as several drops of blood in line with a fingerstick type sample size of blood.

RNA quality and quantity were evaluated from finger stick blood samples with RNA isolated using the PAXgeneRNA extraction versus a TriZol-based method. Mailed patient finger stick samples were stored in PAXgeneRNA buffer at −80° C. 142 samples had RNA extracted with PAXgeneRNA extraction with low volume washes, while 13 samples were thawed and mixed with 700 μl Trizol-LS and 250 μl chloroform. After centrifugation, the top layer was precipitated with isopropanol and glycogen and washed with 80% cold ethanol, centrifuged and the pellet was dried, resuspended in PBS and then purified using the Roche High Pure Isolation kit. RNA integrity and quality were both significantly reduced using Trizol and chloroform extraction versus the PAXgene RNA system. The Trizol reagent system utilizes guanidinium thiocyanate and phenol, and an organic extraction via phenol/chloroform.

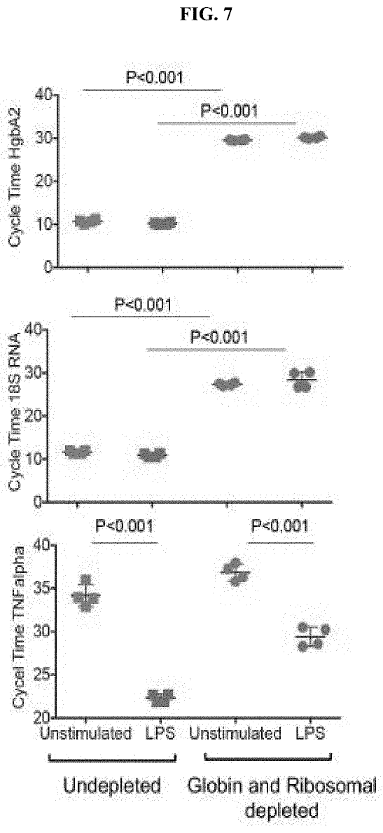

Since ribosomal and hemoglobin RNA represent approximately 98% and 70% of the RNA in whole blood, respectively, we tested standard commercial kits for removing these RNAs prior to RNAseq. The PAXgene system does not remove globin mRNA, which can constitute up to 70% of the mRNA mass in whole blood total RNA. GlobinZero (Illumina) method and kit was utilized to remove globin mRNA from the samples. 4 ml heparinized blood was treated with 1 ug/ml LPS for one hour at 37° C. and 250 ul blood was placed into 250 μl PAXgene fixative in replicate microtainer tubes. After RNA extraction, samples were either treated with the globin zero depletion kit (globin and ribosomal depleted) or undepleted and then quantitative PCR was performed to test for hemoglobin A2, 18S RNA, or TNF alpha mRNA expression. FIG. 7 depicts Cycle Times for HbgA2, 18S RNA, and TNF alpha after GlobinZero depletion. GlobinZero kits depleted both hemoglobin A2 and 18S ribosomal RNA (increased mean cycle time from 11 to 28 and 10 to 30, respectively) with relative preservation of TNFalpha mRNA.

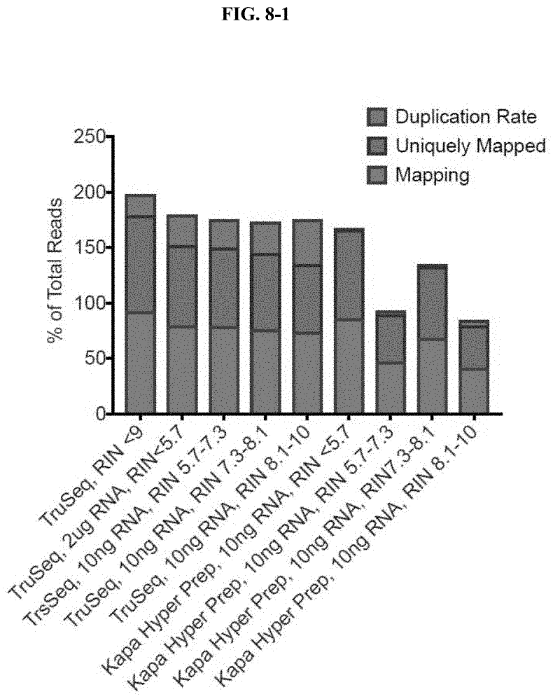

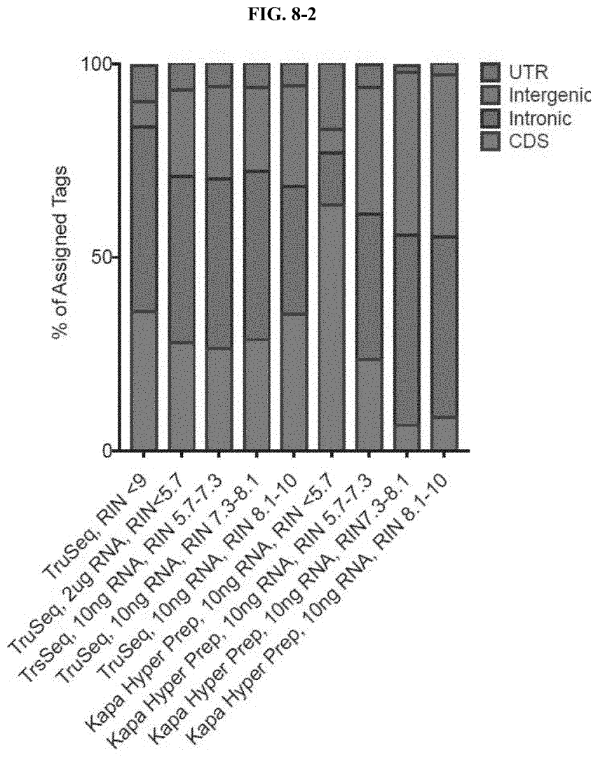

RNASeq QC metrics were assessed on RNA prepared with Illumina TruSeq or Kapa Hyper Prep Kits and having various RIN scores ranging from <5.7 to 8.1-10 (FIG. 8). Distribution of mapping, uniquely mapping, and duplicate reads was plotted for the TruSeq and Kapa Hyper Prep RNAs with various RIN scores. Distribution of tags assigned to UTR (untranslated region), intergenic, intronic, and CDS (coding sequence) of whole blood RNA samples prepared with Illumina TruSeq or Kapa Hyper Prep Kits with various input RNA quality and quantity was determined. The Illumina TruSeq library Prep demonstrated increased mapping to coding sequence and fewer intergenic reads and was ultimately used for downstream experiments.

To assess the validity of patient reported disease activity, we compared their RAPID3 scores with clinician collected DAS28. Significant correlations were evident between RAPID3 and DAS28 for each of the four patients (FIG. 1B). To assess the validity of fingerstick blood data, we compared RNAseq inferred white blood cell counts with clinical laboratory measurements of complete blood counts and again observed significant correlations (FIG. 1C). Taken together, these data indicate that patient reports of disease activity paired with fingerstick blood samples provide a high quality and robust means by which individuals can participate in longitudinal clinical research studies.

Clinical and Molecular Features of RA Flare Compared to Baseline

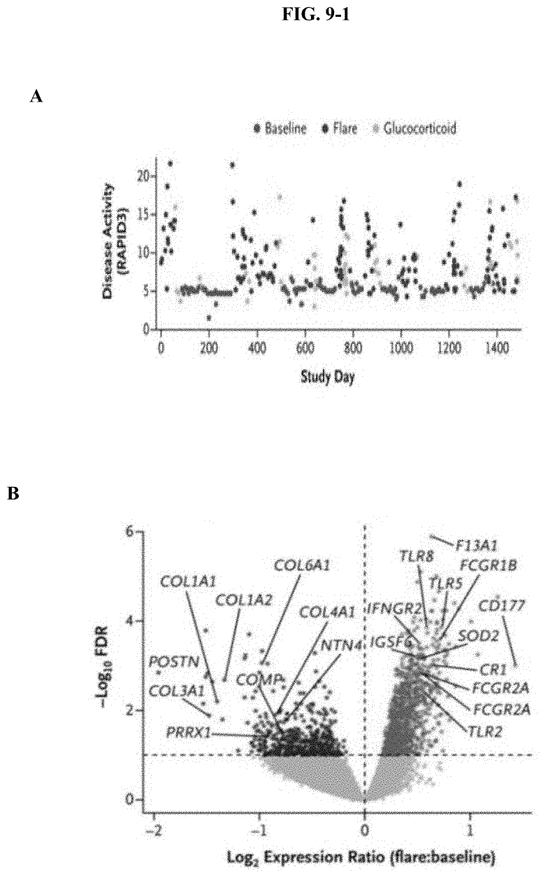

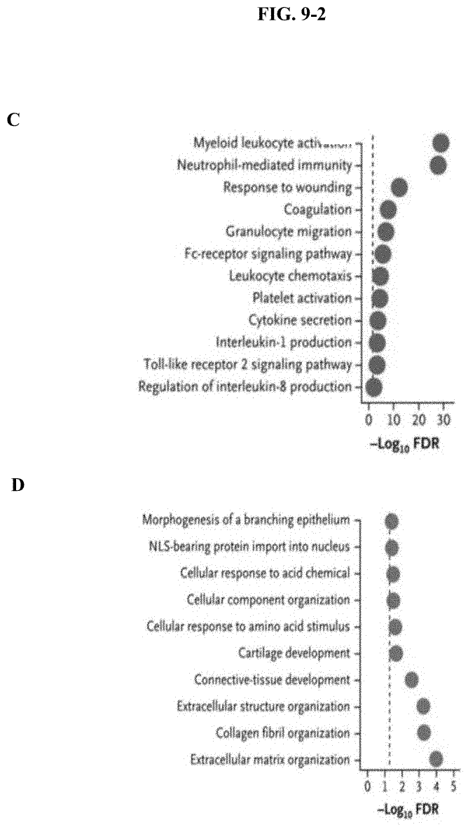

Flares were associated with increases in objective clinical and laboratory measures of RA related disease activity in the index patient (FIG. 9A). Fingerstick RNAseq identified 2613 genes differentially expressed at flare versus baseline (FDR<0.1), with 1437 increased during flare (logFC>0; FIG. 9B). Pathway analysis identified enrichment in myeloid, neutrophil, Fc receptor signaling and platelet activation (FIG. 9C), consistent with clinical CBC measurements during flares. Interestingly, 1176 genes were significantly decreased during flare, and pathway analysis of these genes were enriched for extracellular matrix, collagen and connective tissue development (FIG. 9D).

Time Series Analysis of Molecular Events Leading to RA Flares

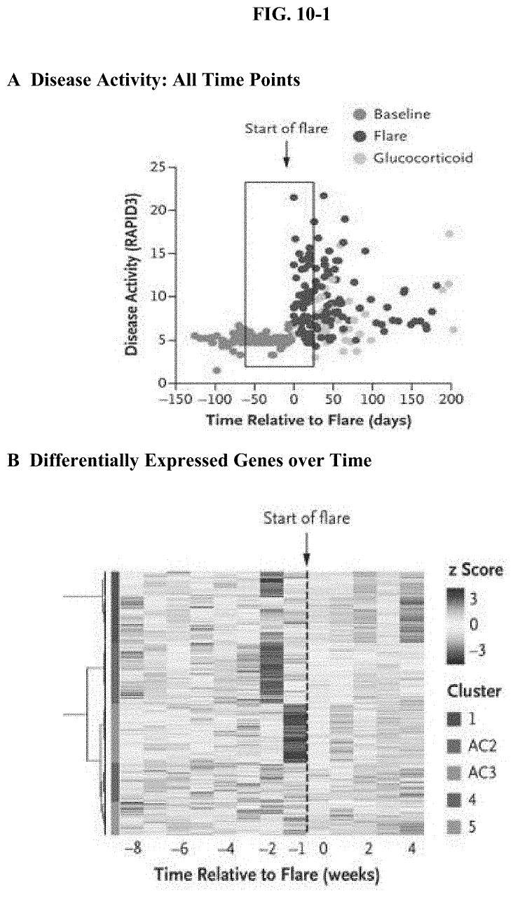

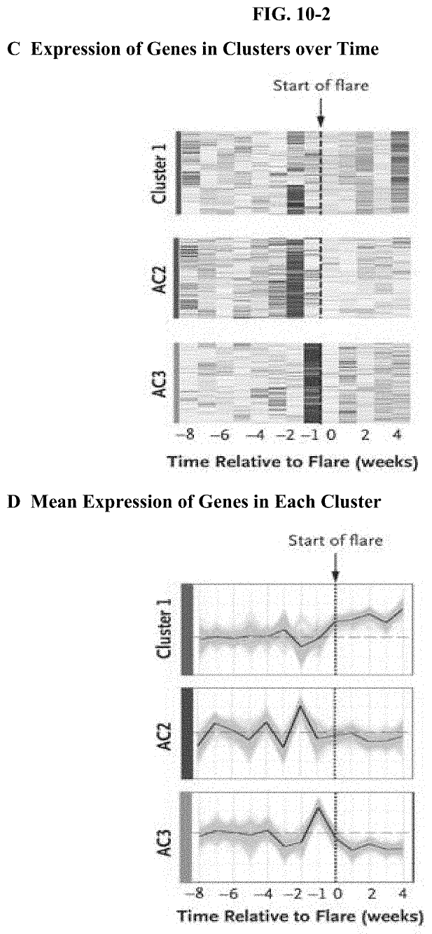

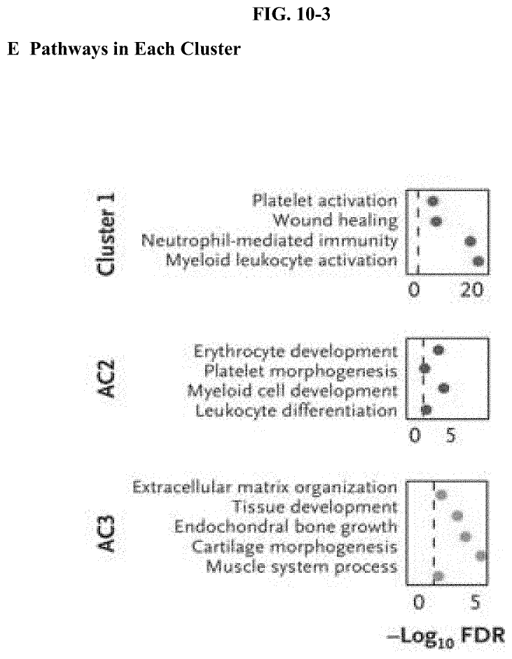

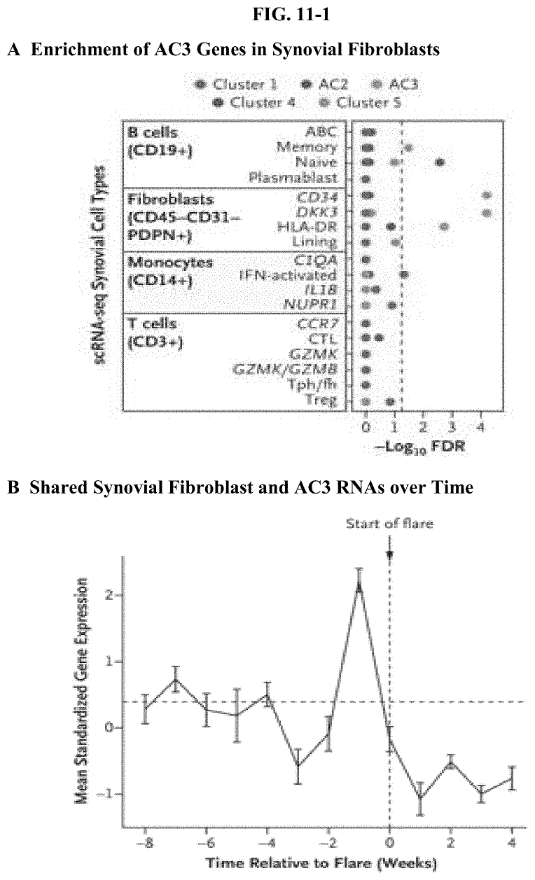

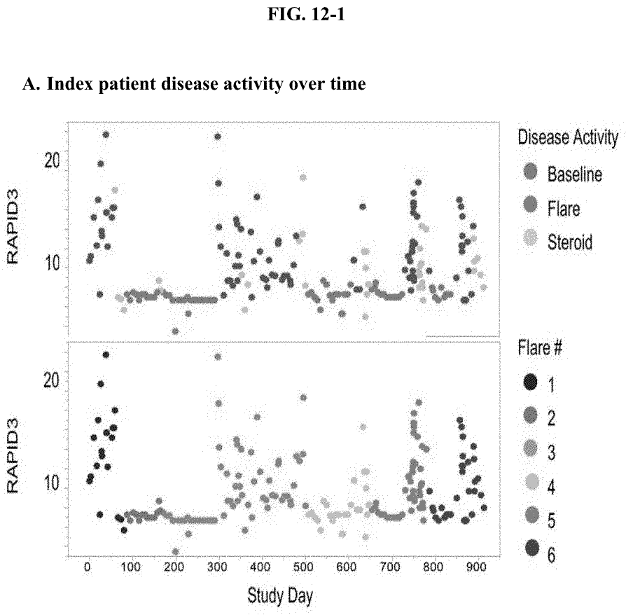

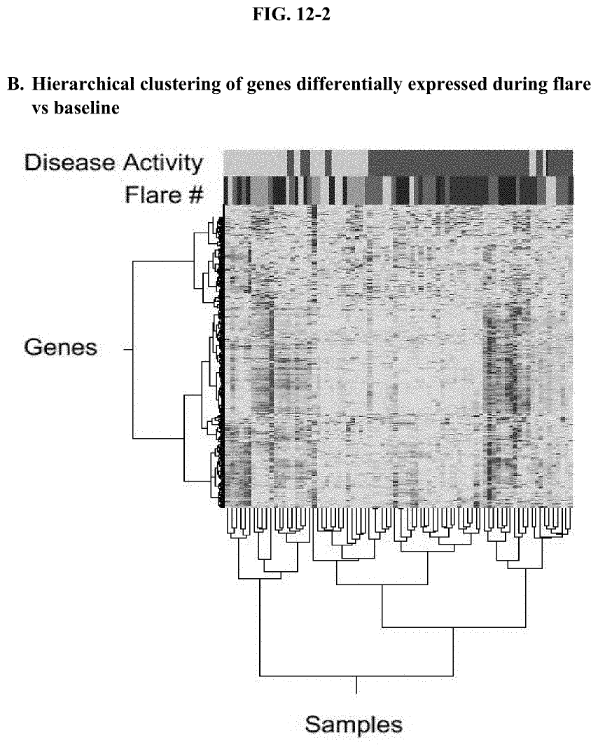

To analyze the trajectories of gene expression over time and identify potential antecedents to flare, we performed time series analysis of the RNAseq data (FIG. 10A). Notably, disease activity scores in the weeks just prior to flare were the same as baseline scores two months prior to flare, underscoring the challenges of identifying both a time frame and gene expression signature that is antecedent to flare. We focused the analysis on 65 samples acquired 8 weeks prior to flare and 4 weeks after flare initiation, binning samples according to the week they were drawn. This identified 2791 genes with significant differential expression over time to flare (FDR<0.05), and hierarchical clustering of gene expression identified five clusters (FIG. 10B). Cluster 1 represented a group of genes which increased after symptom onset (FIGS. 10C and 10D) and was highly overlapping (FIG. 10E) with genes increased in the flare versus baseline analysis (FIG. 9B). These gene expression clusters were reproducibly altered in 5 separate clinical flare events (FIG. 12).

We further focused on two clusters that were differentially expressed antecedent to flare (FIG. 10C-10D). Antecedent cluster 2 (AC2) (Table 2) transcripts increased two weeks prior to flare and were enriched with developmental pathways for naive B cells and leukocytes. Two additional means of deconvoluting the RNAseq data, CIBERSORTx and ABIS, independently confirmed evidence of B cell and T cell populations antecedent to flare, and all analyses showed evidence of innate inflammatory signatures (neutrophils and monocytes) during flare (data not shown).

Antecedent cluster 3 (AC3) (Table 3) transcripts increased the week prior to flare and then decreased for the duration of flare (FIGS. 10C and 10D). AC3 was enriched for pathways not typical of blood samples, including cartilage morphogenesis, endochondral bone growth, and extracellular matrix organization (FIG. 10E), suggesting the presence of an uncharacterized cell type.

Time Series Analysis of Synovial Cell Marker Genes in RA Flares

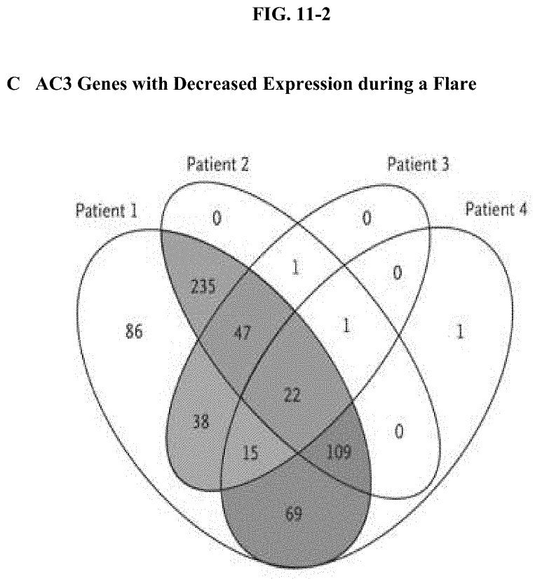

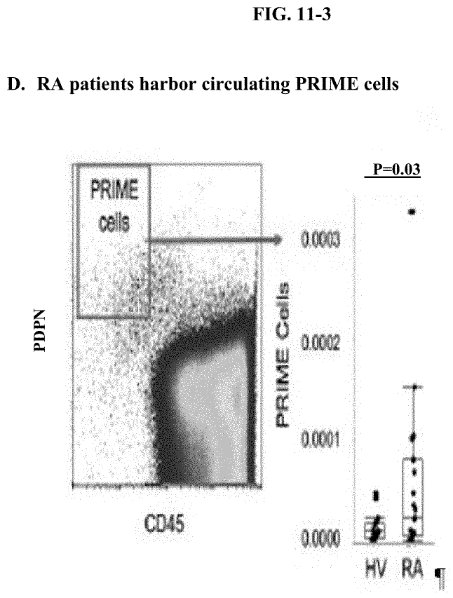

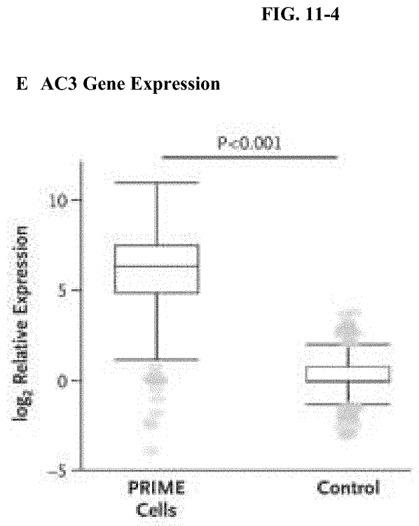

To better characterize the relevance of the clusters identified by the time series analysis to synovitis (FIG. 10C), we examined them for enrichment in synovial cell subtypes characterized by scRNAseq. This analysis of 5265 single RA and osteoarthritis patient synovial cells identified four fibroblast, four B cell, six T cell, and four monocyte subpopulations (FIG. 11A). We identified approximately 200 marker genes that best distinguished each of 18 synovial cell types. AC2 was enriched with naive B cell genes (FIG. 11A), and AC3 was enriched with three sublining fibroblast genes (CD34+, HLA-DR+, and DKK3+) (FIG. 11A). Two of these fibroblast subsets, CD34+ and HLA-DR+, are more abundant in inflamed synovium (20). We plotted expression of those transcripts that were common to both synovial sublining fibroblasts and AC3 over time and again noted their increased expression in blood one week prior to flare and decreased expression during flare (FIG. 11B and Table 1).

Overall, 622 of 625 AC3 genes decreased during flare in patient 1, and a subset (194 genes) also decreased in flares from at least 3 out of 4 RA patients (and 22 genes in 4 out of 4 patients; FIG. 11C), and permutation test indicated this overlap was greater than expected by chance (p=0.0001). Pathway analysis of the subset of 194 overlapping genes was again enriched for extracellular matrix and secreted glycoprotein.

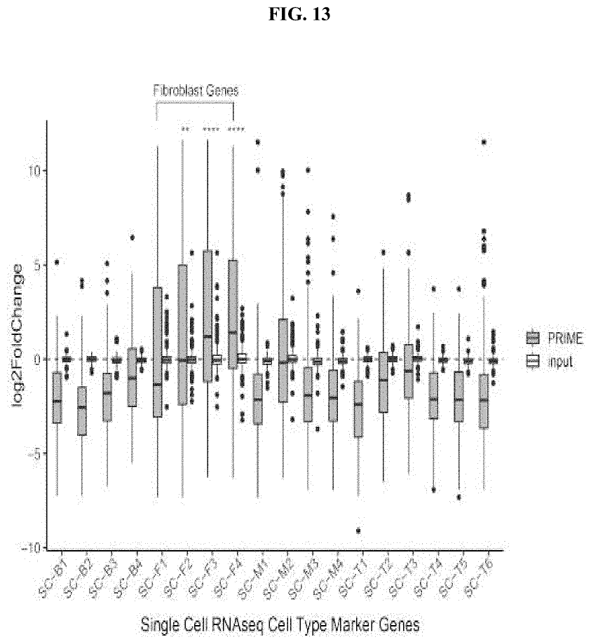

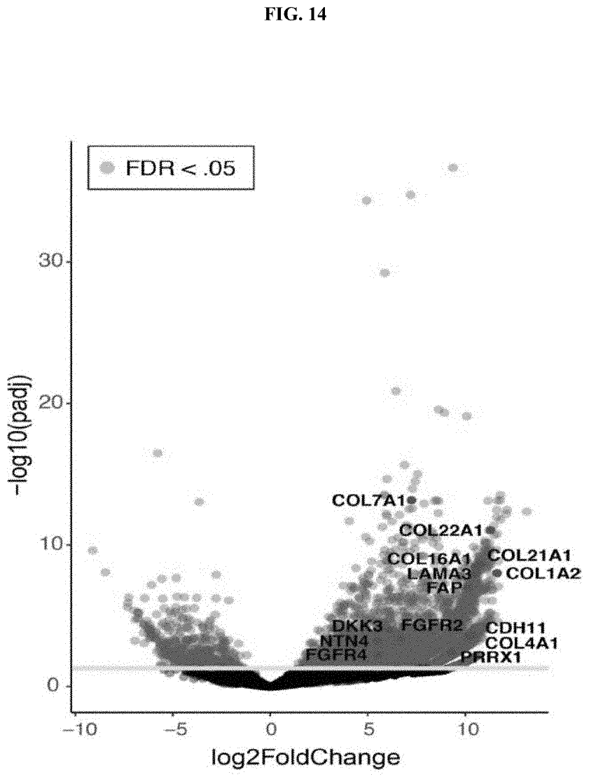

We further tested whether cells that expressed surface markers of synovial fibroblasts were detectable in RA blood by flow cytometry. CD45−/CD31−/PDPN+ cells were increased in 19 additional RA patient blood relative to healthy controls (FIG. 11D). RNAseq of these cells confirmed they were enriched with AC3 cluster genes (FIG. 11E), synovial fibroblast genes (FIG. 13), and expressed classic synovial fibroblast genes such as FAP, DKK3, CDH11, as well as collagens and laminins (FIG. 14). Given their expression of classical mesenchymal surface markers and genes, we refer to these as PRe-Inflammatory Mesenchymal Cells (PRIME cells). Taken together, our observations suggest a model in which sequential activation of B cells activate PRIME cells just prior to flares, which are then evident at flare in inflamed synovium as inflammatory sublining fibroblasts.

The referenced TABLE 1 is provided below:

TABLE 1
GENES COMMON TO SYNOVIAL SUBLINING FIBROBLASTS AND AC3

| cluster | geneset | Ensemble | symbol | auc | pct_nonzero | pct_nonzero_other |
|---|---|---|---|---|---|---|
| SC-F1 | Fibroblast-CD34 + sublining (SC-F1) | ENSG00000187955 | COL14A1 | 0.88699058 | 0.98347107 | 0.30535427 |
| SC-F1 | Fibroblast-CD34 + sublining (SC-F1) | ENSG00000164692 | COL1A2 | 0.84468179 | 1 | 0.53280998 |
| SC-F1 | Fibroblast-CD34 + sublining (SC-F1) | ENSG00000168542 | COL3A1 | 0.82353841 | 1 | 0.53301127 |
| SC-F1 | Fibroblast-CD34 + sublining (SC-F1) | ENSG00000130635 | COL5A1 | 0.76918996 | 0.80991736 | 0.26348631 |
| SC-F1 | Fibroblast-CD34 + sublining (SC-F1) | ENSG00000105664 | COMP | 0.72267741 | 0.5268595 | 0.09178744 |
| SC-F1 | Fibroblast-CD34 + sublining (SC-F1) | ENSG00000133083 | DCLK1 | 0.78673115 | 0.70041322 | 0.16807568 |
| SC-F1 | Fibroblast-CD34 + sublining (SC-F1) | ENSG00000146648 | EGFR | 0.76375622 | 0.72933884 | 0.23007246 |
| SC-F1 | Fibroblast-CD34 + sublining (SC-F1) | ENSG00000120738 | EGR1 | 0.75381699 | 0.95041322 | 0.6507649 |
| SC-F1 | Fibroblast-CD34 + sublining (SC-F1) | ENSG00000164694 | FNDC1 | 0.7526103 | 0.63842975 | 0.15116747 |
| SC-F1 | Fibroblast-CD34 + sublining (SC-F1) | ENSG00000131386 | GALNT15 | 0.7329481 | 0.59297521 | 0.14351852 |
| SC-F1 | Fibroblast-CD34 + sublining (SC-F1) | ENSG00000168079 | SCARA5 | 0.74916074 | 0.78512397 | 0.28965378 |
| SC-F1 | Fibroblast-CD34 + sublining (SC-F1) | ENSG00000137573 | SULF1 | 0.73367922 | 0.67561983 | 0.23389694 |
| SC-F1 | Fibroblast-CD34 + sublining (SC-F1) | ENSG00000091656 | ZFHX4 | 0.77838039 | 0.71487603 | 0.2071256 |
| SC-F2 | Fibroblast-HLA-DRAhi sublining (SC-F2) | ENSG00000187955 | COL14A1 | 0.91760718 | 0.99165508 | 0.27044158 |
| SC-F2 | Fibroblast-HLA-DRAhi sublining (SC-F2) | ENSG00000084636 | COL16A1 | 0.87323477 | 0.83588317 | 0.10585252 |
| SC-F2 | Fibroblast-HLA-DRAhi sublining (SC-F2) | ENSG00000164692 | COL1A2 | 0.92139483 | 1 | 0.50961335 |
| SC-F2 | Fibroblast-HLA-DRAhi sublining (SC-F2) | ENSG00000168542 | COL3A1 | 0.93074592 | 1 | 0.50982464 |
| SC-F2 | Fibroblast-HLA-DRAhi sublining (SC-F2) | ENSG00000134871 | COL4A2 | 0.84125266 | 0.86648122 | 0.18761885 |
| SC-F2 | Fibroblast-HLA-DRAhi sublining (SC-F2) | ENSG00000130635 | COL5A1 | 0.88489277 | 0.95132128 | 0.21487429 |
| SC-F2 | Fibroblast-HLA-DRAhi sublining (SC-F2) | ENSG00000133083 | DCLK1 | 0.83131283 | 0.79972184 | 0.12655821 |
| SC-F2 | Fibroblast-HLA-DRAhi sublining (SC-F2) | ENSG00000146648 | EGFR | 0.80327353 | 0.82058414 | 0.19142193 |
| SC-F2 | Fibroblast-HLA-DRAhi sublining (SC-F2) | ENSG00000120738 | EGR1 | 0.80507545 | 0.9930459 | 0.62941052 |
| SC-F2 | Fibroblast-HLA-DRAhi sublining (SC-F2) | ENSG00000130508 | PXDN | 0.84330642 | 0.83171071 | 0.16670188 |
| SC-F2 | Fibroblast-HLA-DRAhi sublining (SC-F2) | ENSG00000166444 | ST5 | 0.83349089 | 0.81641168 | 0.15867315 |
| SC-F3 | Fibroblast-DKK3 + sublining (SC-F3) | ENSG00000187955 | COL14A1 | 0.85231287 | 0.96929825 | 0.33920368 |
| SC-F3 | Fibroblast-DKK3 + sublining (SC-F3) | ENSG00000164692 | COL1A2 | 0.90670253 | 1 | 0.55570444 |
| SC-F3 | Fibroblast-DKK3 + sublining (SC-F3) | ENSG00000168542 | COL3A1 | 0.90184724 | 1 | 0.55589587 |
| SC-F3 | Fibroblast-DKK3 + sublining (SC-F3) | ENSG00000130635 | COL5A1 | 0.91728082 | 0.98245614 | 0.28273354 |
| SC-F3 | Fibroblast-DKK3 + sublining (SC-F3) | ENSG00000105664 | COMP | 0.81905082 | 0.71929825 | 0.10470904 |
| SC-F3 | Fibroblast-DKK3 + sublining (SC-F3) | ENSG00000164694 | FNDC1 | 0.8072182 | 0.76315789 | 0.16960184 |
| SC-F3 | Fibroblast-DKK3 + sublining (SC-F3) | ENSG00000131386 | GALNT15 | 0.81963055 | 0.76754386 | 0.15792496 |
| SC-F3 | Fibroblast-DKK3 + sublining (SC-F3) | ENSG00000164294 | GPX8 | 0.80951068 | 0.8245614 | 0.22396631 |
| SC-F3 | Fibroblast-DKK3 + sublining (SC-F3) | ENSG00000167779 | IGFBP6 | 0.83772643 | 0.78947368 | 0.16807044 |
| SC-F3 | Fibroblast-DKK3 + sublining (SC-F3) | ENSG00000130508 | PXDN | 0.83692715 | 0.88157895 | 0.2270291 |
| SC-F3 | Fibroblast-DKK3 + sublining (SC-F3) | ENSG00000168079 | SCARA5 | 0.81990132 | 0.89035088 | 0.3093415 |
| SC-F3 | Fibroblast-DKK3 + sublining (SC-F3) | ENSG00000137573 | SULF1 | 0.83014503 | 0.86403509 | 0.24732006 |
| SC-F3 | Fibroblast-DKK3 + sublining (SC-F3) | ENSG00000091656 | ZFHX4 | 0.78976712 | 0.80263158 | 0.22817764 |

Discussion

We present longitudinal genomics as a strategy to study the antecedents to RA flare that may be generalizable to autoimmune diseases associated with waxing/waning clinical courses. We developed easy-to-use tools for patients to acquire both quantifiable clinical symptoms and molecular data at home over many years. This allowed us to capture data prior to the onset of clinical flares and retrospectively analyze it, identifying different RNA signatures (AC2 (Table 2) and AC3 (Table 3) evident in peripheral blood 1-2 weeks prior to flare.

The RNA signature of AC3 and sorted CD45−/CD31−/PDPN+ circulating cells revealed enrichment for pathways including cartilage morphogenesis, endochondral bone growth, and extracellular matrix organization (FIG. 10E) and strongly overlapped with synovial sublining fibroblasts. We therefore propose antecedent PRIME cells are the precursors to inflammatory sublining fibroblasts previously found adjacent to blood vessels in inflamed RA synovium (21).

Significantly, inflamed sublining fibroblasts are pathogenic in an animal model of arthritis (22). Our discovery that human AC3 genes share molecular characteristics of sublining fibroblasts, together with the observation that these cells spike prior to flare but are less detectable in blood during flare (FIGS. 9 and 10) support a model in which PRIME cells immigrate acutely from blood to the synovium where they contribute to the inflammatory process. This model is consistent with the observation that RA synovial fibroblasts can traffic to cartilage implants and are sufficient to passively transfer synovial inflammation in mice (23). Together our data suggest the mesenchymal signal detected in AC3 prior to flares represent a previously uncharacterized type of trafficking fibroblast that circulates in blood.

In addition, we observed a second RNA signature, AC2, activated in blood prior to the spike in AC3. AC2 bear RNA hallmarks of naive B cells. This finding is reminiscent of recent studies demonstrating autoreactive naive B cells are specifically activated in RA patients (24). While the triggers of these are unknown, infectious (for example bacterial or viral antigens), environmental or endogenous toxins (25-27) could provide a source of either specific antigens or activate pattern recognition receptors.

In conclusion, we demonstrate methods for densely collecting longitudinal clinical and gene expression data that can be used to discover changes in transcriptional profiles in the blood weeks prior to symptom onset. The methods include means and procedures for stabilizing, isolating and analyzing RNA from small volume samples which can be collected by a patient or individual themselves such as by finger stick collection, without the need for medical personnel, and which are applicable to home or field collection, to patients which are compromised or otherwise wherein collection of blood by venipuncture is not reasonable or available, and wherein there is a need for rapid sampling or for periodic sampling over time. This approach led to the identification and characterization of RNA markers and indicators of disease or pathological conditions and also the discovery of PRIME cells, bearing hallmarks of synovial fibroblasts, which are more common in RA patients and increase in blood just prior to flares. In modeling all our data, we suggest that prior to clinical flare, systemic B cell immune activation (detected as AC2) acts on PRIME cells, which traffic to the blood (detected as AC3) and subsequently to the synovial sublining during flares of disease activity.

More generally, application of an efficient self-collection protocol and approach combined with quantitative and qualitative RNA isolation to work in RA and RA patient sampling demonstrates the effectiveness and usefulness of our system. This initial study provides an exemplar of an approach to isolating, evaluating and assessing markers and RNA or protein expression changes and cellular changes which are applicable to disease assessment and evaluation, including in waxing/waning inflammatory disease, suggesting a general strategy relevant to numerous diseases and conditions, including additional disorders such as lupus, multiple sclerosis, and vasculitis.

TABLE 2
AC2 GENES

| Ensembl | Symbol | Description | Score?/AUC? |
|---|---|---|---|
| ENSG00000204632 | HLA-G | “major histocompatibility complex, class I, G | 5.77E−05 |
| | | [Source: HGNC Symbol; Acc: HGNC: 4964]” | |
| ENSG00000184792 | OSBP2 | oxysterol binding protein 2 | 6.39E−05 |
| | | [Source: HGNC Symbol; Acc: HGNC: 8504] | |
| ENSG00000198892 | SHISA4 | shisa family member 4 | 0.00012762 |
| | | [Source: HGNC Symbol; Acc: HGNC: 27139] | |
| ENSG00000187017 | ESPN | espin | 0.000131036 |
| | | [Source: HGNC Symbol; Acc: HGNC: 13281] | |
| ENSG00000233762 | | | 0.000323289 |
| ENSG00000175130 | MARCKSL1 | MARCKS like 1 | 0.000358619 |
| | | [Source: HGNC Symbol; Acc: HGNC: 7142] | |
| ENSG00000125534 | PPDPF | pancreatic progenitor cell differentiation and | 0.00037025 |
| | | proliferation factor | |
| | | [Source: HGNC Symbol; Acc: HGNC: 16142] | |
| ENSG00000158856 | DMTN | dematin actin binding protein | 0.000376535 |
| | | [Source: HGNC Symbol; Acc: HGNC: 3382] | |
| ENSG00000121413 | ZSCAN18 | zinc finger and SCAN domain containing 18 | 0.000443873 |
| | | [Source: HGNC Symbol; Acc: HGNC: 21037] | |
| ENSG00000230715 | | | 0.000528797 |
| ENSG00000215030 | RPL13P12 | ribosomal protein L13 pseudogene 12 | 0.000588454 |
| | | [Source: HGNC Symbol; Acc: HGNC: 35701] | |
| ENSG00000146540 | C7orf50 | chromosome 7 open reading frame 50 | 0.000639272 |
| | | [Source: HGNC Symbol; Acc: HGNC: 22421] | |
| ENSG00000029534 | ANK1 | ankyrin 1 | 0.000697583 |
| | | [Source: HGNC Symbol; Acc: HGNC: 492] | |
| ENSG00000121104 | FAM117A | family with sequence similarity 117 member A | 0.000697583 |
| | | [Source: HGNC Symbol; Acc: HGNC: 24179] | |
| ENSG00000260231 | JHDM1D-AS1 | JHDM1D antisense RNA 1 (head to head) | 0.00070319 |
| | | [Source: HGNC Symbol; Acc: HGNC: 48959] | |
| ENSG00000211895 | IGHA1 | immunoglobulin heavy constant alpha 1 | 0.000707226 |
| | | [Source: HGNC Symbol; Acc: HGNC: 5478] | |
| ENSG00000173581 | CCDC106 | coiled-coil domain containing 106 | 0.000730947 |
| | | [Source: HGNC Symbol; Acc: HGNC: 30181] | |
| ENSG00000008441 | NFIX | nuclear factor I X | 0.000837162 |
| | | [Source: HGNC Symbol; Acc: HGNC: 7788] | |
| ENSG00000105701 | FKBP8 | FK506 binding protein 8 | 0.000994972 |
| | | [Source: HGNC Symbol; Acc: HGNC: 3724] | |
| ENSG00000079308 | TNS1 | tensin 1 | 0.00113151 |
| | | [Source: HGNC Symbol; Acc: HGNC: 11973] | |
| ENSG00000264063 | MIR3687-2 | microRNA 3687-2 | 0.001218296 |
| | | [Source: HGNC Symbol; Acc: HGNC: 50835] | |
| ENSG00000049089 | COL9A2 | collagen type IX alpha 2 chain | 0.001236713 |
| | | [Source: HGNC Symbol; Acc: HGNC: 2218] | |
| ENSG00000126461 | SCAF1 | SR-related CTD associated factor 1 | 0.00126185 |
| | | [Source: HGNC Symbol; Acc: HGNC: 30403] | |
| ENSG00000243679 | | | 0.001292673 |
| ENSG00000169136 | ATF5 | activating transcription factor 5 | 0.001421682 |
| | | [Source: HGNC Symbol; Acc: HGNC: 790] | |
| ENSG00000181588 | MEX3D | mex-3 RNA binding family member D | 0.001421682 |
| | | [Source: HGNC Symbol; Acc: HGNC: 16734] | |
| ENSG00000103257 | SLC7A5 | solute carrier family 7 member 5 | 0.001574737 |
| | | [Source: HGNC Symbol; Acc: HGNC: 11063] | |
| ENSG00000175931 | UBE2O | ubiquitin conjugating enzyme E2 O | 0.001669619 |
| | | [Source: HGNC Symbol; Acc: HGNC: 29554] | |
| ENSG00000065268 | WDR18 | WD repeat domain 18 | 0.001764407 |
| | | [Source: HGNC Symbol; Acc: HGNC: 17956] | |
| ENSG00000130300 | PLVAP | plasmalemma vesicle associated protein | 0.001779673 |
| | | [Source: HGNC Symbol; Acc: HGNC: 13635] | |
| ENSG00000232434 | AJM1 | apical junction component 1 homolog | 0.001937905 |
| | | [Source: HGNC Symbol; Acc: HGNC: 37284] | |
| ENSG00000197256 | KANK2 | KN motif and ankyrin repeat domains 2 | 0.001945859 |
| | | [Source: HGNC Symbol; Acc: HGNC: 29300] | |
| ENSG00000229809 | ZNF688 | zinc finger protein 688 | 0.002041059 |
| | | [Source: HGNC Symbol; Acc: HGNC: 30489] | |
| ENSG00000130433 | CACNG6 | calcium voltage-gated channel auxiliary subunit | 0.002327494 |
| | | gamma 6 | |
| | | [Source: HGNC Symbol; Acc: HGNC: 13625] | |
| ENSG00000126254 | RBM42 | RNA binding motif protein 42 | 0.002342133 |
| | | [Source: HGNC Symbol; Acc: HGNC: 28117] | |
| ENSG00000013306 | SLC25A39 | solute carrier family 25 member 39 | 0.002354141 |
| | | [Source: HGNC Symbol; Acc: HGNC: 24279] | |
| ENSG00000179837 | NA | NA | 0.002482976 |
| ENSG00000265714 | NA | NA | 0.002482976 |
| ENSG00000172460 | PRSS30P | “serine protease 30, pseudogene | 0.002490713 |
| | | [Source: HGNC Symbol; Acc: HGNC: 28753]” | |
| ENSG00000104983 | CCDC61 | coiled-coil domain containing 61 | 0.002565172 |
| | | [Source: HGNC Symbol; Acc: HGNC: 33629] | |
| ENSG00000211898 | IGHD | immunoglobulin heavy constant delta | 0.002565172 |
| | | [Source: HGNC Symbol; Acc: HGNC: 5480] | |
| ENSG00000055118 | KCNH2 | potassium voltage-gated channel subfamily H | 0.002637584 |
| | | member 2 | |
| | | [Source: HGNC Symbol; Acc: HGNC: 6251] | |
| ENSG00000260335 | | | 0.002639596 |
| ENSG00000104903 | LYL1 | “LYL1, basic helix-loop-helix family member | 0.002696079 |
| | | [Source: HGNC Symbol; Acc: HGNC: 6734]” | |
| ENSG00000099958 | DERL3 | derlin 3 | 0.002709927 |
| | | [Source: HGNC Symbol; Acc: HGNC: 14236] | |
| ENSG00000179526 | SHARPIN | SHANK associated RH domain interactor | 0.002709927 |
| | | [Source: HGNC Symbol; Acc: HGNC: 25321] | |
| ENSG00000133069 | TMCC2 | transmembrane and coiled-coil domain family 2 | 0.002940811 |
| | | [Source: HGNC Symbol; Acc: HGNC: 24239] | |
| ENSG00000240342 | RPS2P5 | ribosomal protein S2 pseudogene 5 | 0.002940811 |
| | | [Source: HGNC Symbol; Acc: HGNC: 31386] | |
| ENSG00000264462 | MIR3648-2 | microRNA 3648-2 | 0.003313878 |
| | | [Source: HGNC Symbol; Acc: HGNC: 50843] | |
| ENSG00000256576 | LINC02361 | long intergenic non-protein coding RNA 2361 | 0.003383286 |
| | | [Source: HGNC Symbol; Acc: HGNC: 53283] | |
| ENSG00000007968 | E2F2 | E2F transcription factor 2 [ | 0.003400809 |
| | | [Source: HGNC Symbol; Acc: HGNC: 3114] | |
| ENSG00000141858 | SAMD1 | sterile alpha motif domain containing 1 | 0.003623721 |
| | | [Source: HGNC Symbol; Acc: HGNC: 17958] | |
| ENSG00000126705 | AHDC1 | AT-hook DNA binding motif containing 1 | 0.004035577 |
| | | [Source: HGNC Symbol; Acc: HGNC: 25230] | |
| ENSG00000141456 | PELP1 | “proline, glutamate and leucine rich protein 1 | 0.00422876 |
| | | [Source: HGNC Symbol; Acc: HGNC: 30134]” | |
| ENSG00000159713 | TPPP3 | tubulin polymerization promoting protein family | 0.004525344 |
| | | member 3 | |
| | | [Source: HGNC Symbol; Acc: HGNC: 24162] | |
| ENSG00000104897 | SF3A2 | splicing factor 3a subunit 2 | 0.004539725 |
| | | [Source: HGNC Symbol; Acc: HGNC: 10766] | |
| ENSG00000063245 | EPN1 | epsin 1 | 0.004540184 |
| | | [Source: HGNC Symbol; Acc: HGNC: 21604] | |
| ENSG00000162783 | IER5 | immediate early response 5 | 0.004549021 |
| | | [Source: HGNC Symbol; Acc: HGNC: 5393] | |
| ENSG00000141582 | CBX4 | chromobox 4 | 0.004686524 |
| | | [Source: HGNC Symbol; Acc: HGNC: 1554] | |
| ENSG00000168159 | RNF187 | ring finger protein 187 | 0.004686524 |
| | | [Source: HGNC Symbol; Acc: HGNC: 27146] | |
| ENSG00000136826 | KLF4 | Kruppel like factor 4 | 0.004891089 |
| | | [Source: HGNC Symbol; Acc: HGNC: 6348] | |
| ENSG00000237214 | | | 0.004891089 |
| ENSG00000066336 | SPI1 | Spi-1 proto-oncogene | 0.005082358 |
| | | [Source: HGNC Symbol; Acc: HGNC: 11241] | |
| ENSG00000172270 | BSG | basigin (Ok blood group) | 0.005082358 |
| | | [Source: HGNC Symbol; Acc: HGNC: 1116] | |
| ENSG00000173868 | PHOSPHO1 | phosphoethanolamine/phosphocholine phosphatase | 0.005082358 |
| | | [Source: HGNC Symbol; Acc: HGNC: 16815] | |
| ENSG00000167182 | SP2 | Sp2 transcription factor | 0.005118791 |
| | | [Source: HGNC Symbol; Acc: HGNC: 11207] | |
| ENSG00000104805 | NUCB1 | nucleobindin 1 | 0.005119064 |
| | | [Source: HGNC Symbol; Acc: HGNC: 8043] | |
| ENSG00000099381 | SETD1A | SET domain containing 1A | 0.00514547 |
| | | [Source: HGNC Symbol; Acc: HGNC: 29010] | |
| ENSG00000185340 | GAS2L1 | growth arrest specific 2 like 1 | 0.00514547 |
| | | [Source: HGNC Symbol; AcctHGNC: 16955] | |
| ENSG00000007541 | PIGQ | phosphatidylinositol glycan anchor biosynthesis | 0.005166641 |
| | | class Q | |
| | | [Source: HGNC Symbol; Acc: HGNC: 14135] | |
| ENSG00000105610 | KLF1 | Kruppel like factor 1 | 0.005217627 |
| | | [Source: HGNC Symbol; Acc: HGNC: 6345] | |
| ENSG00000137193 | PIM1 | “Pim-1 proto-oncogene, serine/threonine kinase | 0.005247531 |
| | | [Source: HGNC Symbol; Acc: HGNC: 8986]” | |
| ENSG00000171552 | BCL2L1 | BCL2 like 1 | 0.005530506 |
| | | [Source: HGNC Symbol; Acc: HGNC: 992] | |
| ENSG00000172889 | EGFL7 | EGF like domain multiple 7 | 0.005530506 |
| | | [Source: HGNC Symbol; Acc: HGNC: 20594] | |
| ENSG00000213402 | PTPRCAP | “protein tyrosine phosphatase, receptor type C | 0.00560032 |
| | | associated protein | |
| | | [Source: HGNC Symbol; Acc: HGNC: 9667]” | |
| ENSG00000099330 | OCEL1 | occludin/ELL domain containing 1 | 0.005671418 |
| | | [Source: HGNC Symbol; Acc: HGNC: 26221] | |
| ENSG00000147443 | DOK2 | docking protein 2 | 0.005857886 |
| | | [Source: HGNC Symbol; Acc: HGNC: 2991] | |
| ENSG00000182240 | BACE2 | beta-site APP-cleaving enzyme 2 | 0.005857886 |
| | | [Source: HGNC Symbol; Acc: HGNC: 934] | |
| ENSG00000170128 | GPR25 | G protein-coupled receptor 25 | 0.006101348 |
| | | [Source: HGNC Symbol; Acc: HGNC: 4480] | |
| ENSG00000140406 | TLNRD1 | talin rod domain containing 1 | 0.0061541 |
| | | [Source: HGNC Symbol; Acc: HGNC: 13519] | |
| ENSG00000117394 | SLC2A1 | solute carrier family 2 member 1 | 0.006158727 |
| | | [Source: HGNC Symbol; Acc: HGNC: 11005] | |
| ENSG00000141854 | MISP3 | MISP family member 3 | 0.006204755 |
| | | [Source: HGNC Symbol; Acc: HGNC: 26963] | |
| ENSG00000129757 | CDKN1C | cyclin dependent kinase inhibitor 1C | 0.006380434 |
| | | [Source: HGNC Symbol; Acc: HGNC: 1786] | |
| ENSG00000186891 | TNFRSF18 | TNF receptor superfamily member 18 | 0.006421961 |
| | | [Source: HGNC Symbol; Acc: HGNC: 11914] | |
| ENSG00000184897 | H1FX | H1 histone family member X | 0.006465512 |
| | | [Source: HGNC Symbol; Acc: HGNC: 4722] | |
| ENSG00000185236 | RAB11B | “RAB11B, member RAS oncogene family | 0.006602734 |
| | | [Source: HGNC Symbol; Acc: HGNC: 9761]” | |
| ENSG00000030582 | GRN | granulin precursor | 0.006751675 |
| | | [Source: HGNC Symbol; Acc: HGNC: 4601] | |
| ENSG00000071564 | TCF3 | transcription factor 3 | 0.006772165 |
| | | [Source: HGNC Symbol; Acc: HGNC: 11633] | |
| ENSG00000267749 | | | 0.006848491 |
| ENSG00000105373 | NOP53 | NOP53 ribosome biogenesis factor | 0.006952696 |
| | | [Source: HGNC Symbol; Acc: HGNC: 4333] | |
| ENSG00000240445 | FOXO3B | forkhead box O3B pseudogene | 0.006952696 |
| | | [Source: HGNC Symbol; Acc: HGNC: 3822] | |
| ENSG00000127528 | KLF2 | Kruppel like factor 2 | 0.00698206 |
| | | [Source: HGNC Symbol; Acc: HGNC: 6347] | |
| ENSG00000254858 | MPV17L2 | MPV17 mitochondrial inner membrane protein | 0.006997113 |
| | | like 2 | |
| | | [Source: HGNC Symbol; Acc: HGNC: 28177] | |
| ENSG00000130595 | TNNT3 | “troponin T3, fast skeletal type | 0.007133196 |
| | | [Source: HGNC Symbol; Acc: HGNC: 11950]” | |
| ENSG00000130749 | ZC3H4 | zinc finger CCCH-type containing 4 | 0.007141116 |
| | | [Source: HGNC Symbol; Acc: HGNC: 17808] | |
| ENSG00000132819 | RBM38 | RNA binding motif protein 38 | 0.007141116 |
| | | [Source: HGNC Symbol; Acc: HGNC: 15818] | |
| ENSG00000135925 | WNT10A | Wnt family member 10A | 0.007141116 |
| | | [Source: HGNC Symbol; Acc: HGNC: 13829] | |
| ENSG00000205639 | MFSD2B | major facilitator superfamily domain containing 2B | 0.007141116 |
| | | [Source: HGNC Symbol; Acc: HGNC: 37207] | |
| ENSG00000213763 | ACTBP2 | “actin, beta pseudogene 2 | 0.007141116 |
| | | [Source: HGNC Symbol; Acc: HGNC: 135]” | |
| ENSG00000261221 | ZNF865 | zinc finger protein 865 | 0.007141116 |
| | | [Source: HGNC Symbol; Acc: HGNC: 38705] | |
| ENSG00000171611 | PTCRA | pre T cell antigen receptor alpha | 0.007255358 |
| | | [Source: HGNC Symbol; Acc: HGNC: 21290] | |
| ENSG00000161642 | ZNF385A | zinc finger protein 385A | 0.007304748 |
| | | [Source: HGNC Symbol; Acc: HGNC: 17521] | |
| ENSG00000226608 | FTLP3 | ferritin light chain pseudogene 3 | 0.007304748 |
| | | [Source: HGNC Symbol; Acc: HGNC: 4000] | |
| ENSG00000170684 | ZNF296 | zinc finger protein 296 | 0.007327997 |
| | | [Source: HGNC Symbol; Acc: HGNC: 15981] | |
| ENSG00000213638 | ADAT3 | “adenosine deaminase, tRNA specific 3 | 0.007343983 |
| | | [Source: HGNC Symbol; Acc: HGNC: 25151]” | |
| ENSG00000179262 | RAD23A | “RAD23 homolog A, nucleotide excision repair protein | 0.0073448 |
| | | [Source: HGNC Symbol; Acc: HGNC: 9812]” | |
| ENSG00000196126 | HLA-DRB1 | “major histocompatibility complex, class II, DR beta 1 | 0.00745996 |
| | | [Source: HGNC Symbol; Acc: HGNC: 4948]” | |
| ENSG00000197149 | | | 0.007535198 |
| ENSG00000213820 | RPL13P2 | ribosomal protein L13 pseudogene 2 | 0.007619923 |
| | | [Source: HGNC Symbol; Acc: HGNC: 16342] | |
| ENSG00000225331 | LINC01678 | long intergenic non-protein coding RNA 1678 | 0.007619923 |
| | | [Source: HGNC Symbol; Acc: HGNC: 52466] | |
| ENSG00000235605 | | | 0.007619923 |
| ENSG00000183092 | BEGAIN | brain enriched guanylate kinase associated | 0.007755013 |
| | | [Source: HGNC Symbol; Acc: HGNC: 24163] | |
| ENSG00000105369 | CD79A | CD79a molecule | 0.007835589 |
| | | [Source: HGNC Symbol; Acc: HGNC: 1698] | |
| ENSG00000160256 | FAM207A | family with sequence similarity 207 member A | 0.007901726 |
| | | [Source: HGNC Symbol; Acc: HGNC: 15811] | |
| ENSG00000105516 | DBP | D-box binding PAR bZIP transcription factor | 0.00795596 |
| | | [Source: HGNC Symbol; Acc: HGNC: 2697] | |
| ENSG00000179094 | PER1 | period circadian regulator 1 | 0.008010463 |
| | | [Source: HGNC Symbol; Acc: HGNC: 8845] | |
| ENSG00000154146 | NRGN | neurogranin | 0.008019602 |
| | | [Source: HGNC Symbol; Acc: HGNC: 8000] | |
| ENSG00000160813 | PPP1R35 | protein phosphatase 1 regulatory subunit 35 | 0.008019602 |
| | | [Source: HGNC Symbol; Acc: HGNC: 28320] | |
| ENSG00000152082 | MZT2B | mitotic spindle organizing protein 2B | 0.008149414 |
| | | [Source: HGNC Symbol; Acc: HGNC: 25886] | |
| ENSG00000115274 | INO80B | INO80 complex subunit B | 0.008388785 |
| | | [Source: HGNC Symbol; Acc: HGNC: 13324] | |
| ENSG00000185112 | FAM43A | family with sequence similarity 43 member A | 0.008409751 |
| | | [Source: HGNC Symbol; Acc: HGNC: 26888] | |
| ENSG00000130592 | LSP1 | lymphocyte-specific protein 1 | 0.00866593 |
| | | [Source: HGNC Symbol; Acc: HGNC: 6707] | |
| ENSG00000077348 | EXOSC5 | exosome component 5 | 0.008679008 |
| | | [Source: HGNC Symbol; Acc: HGNC: 24662] | |
| ENSG00000196498 | NCOR2 | nuclear receptor corepressor 2 | 0.008729326 |
| | | [Source: HGNC Symbol; Acc: HGNC: 7673] | |
| ENSG00000132382 | MYBBP1A | MYB binding protein 1a | 0.008886691 |
| | | [Source: HGNC Symbol; Acc: HGNC: 7546] | |
| ENSG00000104885 | DOT1L | DOT1 like histone lysine methyltransferase | 0.008912378 |
| | | [Source: HGNC Symbol; Acc: HGNC: 24948] | |
| ENSG00000153443 | UBALD1 | UBA like domain containing 1 | 0.008912378 |
| | | [Source: HGNC Symbol; Acc: HGNC: 29576] | |
| ENSG00000070182 | SPTB | “spectrin beta, erythrocytic | 0.008927471 |
| | | [Source: HGNC Symbol; Acc: HGNC: 11274]” | |
| ENSG00000168517 | HEXIM2 | hexamethylene bisacetamide inducible 2 | 0.008959412 |
| | | [Source: HGNC Symbol; Acc: HGNC: 28591] | |
| ENSG00000090674 | MCOLN1 | mucolipin 1 | 0.009327518 |
| | | [Source: HGNC Symbol; Acc: HGNC: 13356] | |
| ENSG00000198816 | ZNF358 | zinc finger protein 358 | 0.009506451 |
| | | [Source: HGNC Symbol; Acc: HGNC: 16838] | |
| ENSG00000175334 | BANF1 | barrier to autointegration factor 1 | 0.009647997 |
| | | [Source: HGNC Symbol; Acc: HGNC: 17397] | |
| ENSG00000125520 | SLC2A4RG | SLC2A4 regulator | 0.009686596 |
| | | [Source: HGNC Symbol; Acc: HGNC: 15930] | |
| ENSG00000141084 | RANBP10 | RAN binding protein 10 | 0.009715508 |
| | | [Source: HGNC Symbol; Acc: HGNC: 29285] | |
| ENSG00000149016 | TUT1 | “terminal uridylyl transferase 1, U6 snRNA-specific | 0.009768686 |
| | | [Source: HGNC Symbol; Acc: HGNC: 26184]” | |
| ENSG00000178951 | ZBTB7A | zinc finger and BTB domain containing 7A | 0.009810065 |
| | | [Source: HGNC Symbol; Acc: HGNC: 18078] | |
| ENSG00000186111 | PIP5K1C | phosphatidylinositol-4-phosphate 5-kinase type 1 gamma | 0.009810065 |
| | | [Source: HGNC Symbol; Acc: HGNC: 8996] | |
| ENSG00000184481 | FOXO4 | forkhead box 04 | 0.009820284 |
| | | [Source: HGNC Symbol; Acc: HGNC: 7139] | |
| ENSG00000064961 | HMG20B | high mobility group 20B | 0.009858965 |
| | | [Source: HGNC Symbol; Acc: HGNC: 5002] | |
| ENSG00000108309 | RUNDC3A | RUN domain containing 3A | 0.010055443 |
| | | [Source: HGNC Symbol; Acc: HGNC: 16984] | |
| ENSG00000130165 | ELOF1 | elongation factor 1 homolog | 0.010212535 |
| | | [Source: HGNC Symbol; Acc: HGNC: 28691] | |
| ENSG00000130159 | ECSIT | ECSIT signalling integrator | 0.010245658 |
| | | [Source: HGNC Symbol; Acc: HGNC: 29548] | |
| ENSG00000244560 | | | 0.010245658 |
| ENSG00000125148 | MT2A | metallothionein 2A | 0.01061889 |
| | | [Source: HGNC Symbol; Acc: HGNC: 7406] | |
| ENSG00000131116 | ZNF428 | zinc finger protein 428 | 0.010712491 |
| | | [Source: HGNC Symbol; Acc: HGNC: 20804] | |
| ENSG00000105617 | LENG1 | leukocyte receptor cluster member 1 | 0.010999325 |
| | | [Source: HGNC Symbol; Acc: HGNC: 15502] | |
| ENSG00000139718 | SETD1B | SET domain containing IB | 0.011038344 |
| | | [Source: HGNC Symbol; Acc: HGNC: 29187] | |
| ENSG00000106665 | CLIP2 | CAP-Gly domain containing linker protein 2 | 0.011064297 |
| | | [Source: HGNC Symbol; Acc: HGNC: 2586] | |
| ENSG00000130821 | SLC6A8 | solute carrier family 6 member 8 | 0.011213055 |
| | | [Source: HGNC Symbol; Acc: HGNC: 11055] | |
| ENSG00000184232 | OAF | out at first homolog | 0.011286635 |
| | | [Source: HGNC Symbol; Acc: HGNC: 28752] | |
| ENSG00000179820 | MYADM | myeloid associated differentiation marker | 0.011330192 |
| | | [Source: HGNC Symbol; Acc: HGNC: 7544] | |
| ENSG00000127580 | WDR24 | WD repeat domain 24 | 0.011570001 |
| | | [Source: HGNC Symbol; Acc: HGNC: 20852] | |
| ENSG00000004939 | SLC4A1 | solute carrier family 4 member 1 (Diego blood group) | 0.011732314 |
| | | [Source: HGNC Symbol; Acc: HGNC: 11027] | |
| ENSG00000130522 | JUND | “JunD proto-oncogene, AP-1 transcription factor | 0.011813745 |
| | | subunit | |
| | | [Source: HGNC Symbol; Acc: HGNC: 6206]” | |
| ENSG00000148362 | PAXX | “PAXX, non-homologous end joining factor | 0.011821074 |
| | | [Source: HGNC Symbol; Acc: HGNC: 27849]” | |
| ENSG00000262902 | MTCO1P40 | mitochondrially encoded cytochrome c oxidase | 0.011821074 |
| | | I pseudogene 40 | |
| | | [Source: HGNC Symbol; Acc: HGNC: 52105] | |
| ENSG00000167671 | UBXN6 | UBX domain protein 6 | 0.011831088 |
| | | [Source: HGNC Symbol; Acc: HGNC: 14928] | |
| ENSG00000125457 | MIF4GD | MIF4G domain containing | 0.011851589 |
| | | [Source: HGNC Symbol; Acc: HGNC: 24030] | |
| ENSG00000146066 | HIGD2A | HIG1 hypoxia inducible domain family member 2A | 0.011914767 |
| | | [Source: HGNC Symbol; Acc: HGNC: 28311] | |
| ENSG00000184221 | OLIG1 | oligodendrocyte transcription factor 1 | 0.011914767 |
| | | [Source: HGNC Symbol; Acc: HGNC: 16983] | |
| ENSG00000260316 | | | 0.0119947 |
| ENSG00000124762 | CDKN1A | cyclin dependent kinase inhibitor 1A | 0.012077375 |
| | | [Source: HGNC Symbol; Acc: HGNC: 1784] | |
| ENSG00000103148 | NPRL3 | “NPR3 like, GATOR1 complex subunit | 0.012137266 |
| | | [Source: HGNC Symbol; Acc: HGNC: 14124]” | |
| ENSG00000179115 | FARSA | phenylalanyl-tRNA synthetase alpha subunit | 0.012137266 |
| | | [Source: HGNC Symbol; Acc: HGNC: 3592] | |
| ENSG00000120896 | SORBS3 | sorbin and SH3 domain containing 3 | 0.012150664 |
| | | [Source: HGNC Symbol; Acc: HGNC: 30907] | |
| ENSG00000174886 | NDUFA11 | NADH: ubiquinone oxidoreductase subunit A11 | 0.012268883 |
| | | [Source: HGNC Symbol; Acc: HGNC: 20371] | |
| ENSG00000102145 | GATA1 | GATA binding protein 1 | 0.012285964 |
| | | [Source: HGNC Symbol; Acc: HGNC: 4170] | |
| ENSG00000166428 | PLD4 | phospholipase D family member 4 | 0.012401569 |
| | | [Source: HGNC Symbol; Acc: HGNC: 23792] | |
| ENSG00000213015 | ZNF580 | zinc finger protein 580 | 0.012630003 |
| | | [Source: HGNC Symbol; Acc: HGNC: 29473] | |
| ENSG00000142544 | CTU1 | cytosolic thiouridylase subunit 1 | 0.012676606 |
| | | [Source: HGNC Symbol; Acc: HGNC: 29590] | |
| ENSG00000085644 | ZNF213 | zinc finger protein 213 | 0.012935817 |
| | | [Source: HGNC Symbol; Acc: HGNC: 13005] | |
| ENSG00000003249 | DBNDD1 | dysbindin domain containing 1 | 0.013109402 |
| | | [Source: HGNC Symbol; Acc: HGNC: 28455] | |
| ENSG00000221288 | MIR663B | microRNA 663b | 0.013333238 |
| | | [Source: HGNC Symbol; Acc: HGNC: 35270] | |
| ENSG00000042062 | RIPOR3 | RIPOR family member 3 | 0.013356858 |
| | | [Source: HGNC Symbol; Acc: HGNC: 16168] | |
| ENSG00000105329 | TGFB1 | transforming growth factor beta 1 | 0.013356858 |
| | | [Source: HGNC Symbol; Acc: HGNC: 11766] | |
| ENSG00000116871 | MAP7D1 | MAP7 domain containing 1 | 0.013356858 |
| | | [Source: HGNC Symbol; Acc: HGNC: 25514] | |
| ENSG00000168298 | HIST1H1E | histone cluster 1 H1 family member e | 0.013400191 |
| | | [Source: HGNC Symbol; Acc: HGNC: 4718] | |
| ENSG00000127666 | TICAM1 | toll like receptor adaptor molecule 1 | 0.013447765 |
| | | [Source: HGNC Symbol; Acc: HGNC: 18348] | |
| ENSG00000166886 | NAB2 | NGFI-A binding protein 2 | 0.013572419 |
| | | [Source: HGNC Symbol; Acc: HGNC: 7627] | |
| ENSG00000112787 | FBRSL1 | fibrosin like 1 | 0.013760569 |
| | | [Source: HGNC Symbol; Acc: HGNC: 29308] | |
| ENSG00000100243 | CYB5R3 | cytochrome b5 reductase 3 | 0.013883197 |
| | | [Source: HGNC Symbol; Acc: HGNC: 2873] | |
| ENSG00000197457 | STMN3 | stathmin 3 | 0.013964273 |
| | | [Source: HGNC Symbol; Acc: HGNC: 15926] | |
| ENSG00000255441 | | | 0.013965194 |
| ENSG00000173801 | JUP | junction plakoglobin | 0.014437619 |
| | | [Source: HGNC Symbol; Acc: HGNC: 6207] | |
| ENSG00000224614 | TNK2-AS1 | TNK2 antisense RNA 1 | 0.014437619 |
| | | [Source: HGNC Symbol; Acc: HGNC: 49093] | |
| ENSG00000058453 | CROCC | “ciliary rootlet coiled-coil, rootletin | 0.014522615 |
| | | [Source: HGNC Symbol; Acc: HGNC: 21299]” | |
| ENSG00000079313 | REXO1 | RNA exonuclease 1 homolog | 0.014579462 |
| | | [Source: HGNC Symbol; Acc: HGNC: 24616] | |
| ENSG00000154102 | C16orf74 | chromosome 16 open reading frame 74 | 0.014732236 |
| | | [Source: HGNC Symbol; Acc: HGNC: 23362] | |
| ENSG00000172650 | AGAP5 | “ArfGAP with GTPase domain, ankyrin repeat | 0.014761715 |
| | | and PH domain 5 | |
| | | [Source: HGNC Symbol; Acc: HGNC: 23467]” | |
| ENSG00000159733 | ZFYVE28 | zinc finger FYVE-type containing 28 | 0.014792652 |
| | | [Source: HGNC Symbol; Acc: HGNC: 29334] | |
| ENSG00000019582 | CD74 | CD74 molecule | 0.014946895 |
| | | [Source: HGNC Symbol; Acc: HGNC: 1697] | |
| ENSG00000211771 | TRBJ2-7 | T cell receptor beta joining 2-7 | 0.014961068 |
| | | [Source: HGNC Symbol; Acc: HGNC: 12175] | |
| ENSG00000214309 | MBLAC1 | metallo-beta-lactamase domain containing 1 | 0.014976932 |
| | | [Source: HGNC Symbol; Acc: HGNC: 22180] | |
| ENSG00000187266 | EPOR | erythropoietin receptor | 0.015342955 |
| | | [Source: HGNC Symbol; Acc: HGNC: 3416] | |
| ENSG00000108106 | UBE2S | ubiquitin conjugating enzyme E2 S | 0.015459542 |
| | | [Source: HGNC Symbol; Acc: HGNC: 17895] | |
| ENSG00000185838 | GNB1L | G protein subunit beta 1 like | 0.015552452 |
| | | [Source: HGNC Symbol; Acc: HGNC: 4397] | |
| ENSG00000228594 | FNDC10 | fibronectin type III domain containing 10 | 0.015552452 |
| | | [Source: HGNC Symbol; Acc: HGNC: 42951] | |
| ENSG00000126464 | PRR12 | proline rich 12 | 0.015622838 |
| | | [Source: HGNC Symbol; Acc: HGNC: 29217] | |
| ENSG00000084092 | NOA1 | nitric oxide associated 1 | 0.015753463 |
| | | [Source: HGNC Symbol; Acc: HGNC: 28473] | |
| ENSG00000105227 | PRX | periaxin | 0.015787169 |
| | | [Source: HGNC Symbol; Acc: HGNC: 13797] | |
| ENSG00000260401 | | | 0.015787169 |
| ENSG00000159840 | ZYX | zyxin | 0.015829354 |
| | | [Source: HGNC Symbol; Acc: HGNC: 13200] | |
| ENSG00000197483 | ZNF628 | zinc finger protein 628 | 0.015834462 |
| | | [Source: HGNC Symbol; Acc: HGNC: 28054] | |
| ENSG00000182572 | NA | NA | 0.015908819 |
| ENSG00000154035 | NA | NA | 0.015942034 |
| ENSG00000161618 | ALDH16A1 | aldehyde dehydrogenase 16 family member A1 | 0.015942034 |
| | | [Source: HGNC Symbol; Acc: HGNC: 28114] | |
| ENSG00000124575 | HIST1H1D | histone cluster 1 H1 family member d | 0.015948868 |
| | | [Source: HGNC Symbol; Acc: HGNC: 4717] | |
| ENSG00000196092 | PAX5 | paired box 5 | 0.01597311 |
| | | [Source: HGNC Symbol; Acc: HGNC: 8619] | |
| ENSG00000105429 | MEGF8 | multiple EGF like domains 8 | 0.015986308 |
| | | [Source: HGNC Symbol; Acc: HGNC: 3233] | |
| ENSG00000213753 | CENPBD1P1 | CENPB DNA-binding domains containing 1 | 0.016008143 |
| | | pseudogene 1 | |
| | | [Source: HGNC Symbol; Acc: HGNC: 28421] | |
| ENSG00000179627 | ZBTB42 | zinc finger and BTB domain containing 42 | 0.016166469 |
| | | [Source: HGNC Symbol; Acc: HGNC: 32550] | |
| ENSG00000107816 | LZTS2 | leucine zipper tumor suppressor 2 | 0.016243979 |
| | | [Source: HGNC Symbol; Acc: HGNC: 29381] | |
| ENSG00000183779 | ZNF703 | zinc finger protein 703 | 0.016243979 |
| | | [Source: HGNC Symbol; Acc: HGNC: 25883] | |
| ENSG00000203950 | RTL8A | retrotransposon Gag like 8A | 0.01630694 |
| | | [Source: HGNC Symbol; Acc: HGNC: 24514] | |
| ENSG00000088826 | SMOX | spermine oxidase | 0.016416472 |
| | | [Source: HGNC Symbol; Acc: HGNC: 15862] | |
| ENSG00000105298 | CACTIN | “cactin, spliceosome C complex subunit | 0.016416472 |
| | | [Source: HGNC Symbol; Acc: HGNC: 29938]” | |
| ENSG00000137218 | FRS3 | fibroblast growth factor receptor substrate 3 | 0.016416472 |
| | | [Source: HGNC Symbol; Acc: HGNC: 16970] | |
| ENSG00000175550 | DRAP1 | DR1 associated protein 1 | 0.016587059 |
| | | [Source: HGNC Symbol; Acc: HGNC: 3019] | |
| ENSG00000166165 | CKB | creatine kinase B | 0.01659925 |
| | | [Source: HGNC Symbol; Acc: HGNC: 1991] | |
| ENSG00000162366 | PDZK1IP1 | PDZK1 interacting protein 1 | 0.016705327 |
| | | [Source: HGNC Symbol; Acc: HGNC: 16887] | |
| ENSG00000184428 | TOP1MT | DNA topoisomerase I mitochondrial | 0.016722415 |
| | | [Source: HGNC Symbol; Acc: HGNC: 29787] | |
| ENSG00000130479 | MAP1S | microtubule associated protein IS | 0.016796937 |
| | | [Source: HGNC Symbol; Acc: HGNC: 15715] | |
| ENSG00000171222 | SCAND1 | SCAN domain containing 1 | 0.016821415 |
| | | [Source: HGNC Symbol; Acc: HGNC: 10566] | |
| ENSG00000171223 | JUNB | “JunB proto-oncogene, AP-1 transcription | 0.016966042 |
| | | factor subunit | |
| | | [Source: HGNC Symbol; Acc: HGNC: 6205]” | |
| ENSG00000107902 | LHPP | phospholysine phosphohistidine inorganic | 0.017052413 |
| | | pyrophosphate phosphatase | |
| | | [Source: HGNC Symbol; Acc: HGNC: 30042] | |
| ENSG00000170271 | FAXDC2 | fatty acid hydroxylase domain containing 2 | 0.017052413 |
| | | [Source: HGNC Symbol; Acc: HGNC: 1334] | |
| ENSG00000100325 | ASCC2 | activating signal cointegrator 1 complex subunit 2 | 0.017132234 |
| | | [Source: HGNC Symbol; Acc: HGNC: 24103] | |
| ENSG00000142694 | EVA1B | eva-1 homolog B | 0.017132234 |
| | | [Source: HGNC Symbol; Acc: HGNC: 25558] | |
| ENSG00000064201 | TSPAN32 | tetraspanin 32 | 0.017210927 |
| | | [Source: HGNC Symbol; Acc: HGNC: 13410] | |
| ENSG00000157911 | PEX10 | peroxisomal biogenesis factor 10 | 0.017211726 |
| | | [Source: HGNC Symbol; Acc: HGNC: 8851] | |
| ENSG00000079432 | CIC | capicua transcriptional repressor | 0.017339051 |
| | | [Source: HGNC Symbol; Acc: HGNC: 14214] | |
| ENSG00000188825 | LINC00910 | long intergenic non-protein coding RNA 910 | 0.017339051 |
| | | [Source: HGNC Symbol; Acc: HGNC: 44361] | |
| ENSG00000196961 | AP2A1 | adaptor related protein complex 2 alpha 1 subunit | 0.017339051 |
| | | [Source: HGNC Symbol; Acc: HGNC: 561] | |
| ENSG00000214279 | SCART1 | scavenger receptor family member expressed on | 0.017339051 |
| | | T cells 1 | |
| | | [Source: HGNC Symbol; Acc: HGNC: 32411] | |
| ENSG00000272449 | | | 0.017339051 |
| ENSG00000104973 | MED25 | mediator complex subunit 25 | 0.017392388 |
| | | [Source: HGNC Symbol; Acc: HGNC: 28845] | |
| ENSG00000180767 | CHST13 | carbohydrate sulfotransferase 13 | 0.017392388 |
| | | [Source: HGNC Symbol; Acc: HGNC: 21755] | |
| ENSG00000227232 | WASH7P | WAS protein family homolog 7 pseudogene | 0.017392388 |
| | | [Source: HGNC Symbol; Acc: HGNC: 38034] | |
| ENSG00000162302 | RPS6KA4 | ribosomal protein S6 kinase A4 | 0.017767827 |
| | | [Source: HGNC Symbol; Acc: HGNC: 10433] | |
| ENSG00000136840 | ST6GALNAC4 | “ST6 N-acetylgalactosaminide | 0.017832974 |
| | | alpha-2,6-sialyltransferase 4 | |
| | | [Source: HGNC Symbol; Acc: HGNC: 17846]” | |
| ENSG00000160404 | TOR2A | torsin family 2 member A | 0.018020454 |
| | | [Source: HGNC Symbol; Acc: HGNC: 11996] | |
| ENSG00000233038 | | | 0.018071818 |
| ENSG00000243449 | C4orf48 | chromosome 4 open reading frame 48 | 0.018116836 |
| | | [Source: HGNC Symbol; Acc: HGNC: 34437] | |
| ENSG00000160050 | CCDC28B | coiled-coil domain containing 28B | 0.01812987 |
| | | [Source: HGNC Symbol; Acc: HGNC: 28163] | |
| ENSG00000138623 | SEMA7A | semaphorin 7A (John Milton Hagen blood group) | 0.01834001 |
| | | [Source: HGNC Symbol; Acc: HGNC: 10741] | |
| ENSG00000101439 | CST3 | cystatin C | 0.018421259 |
| | | [Source: HGNC Symbol; Acc: HGNC: 2475] | |
| ENSG00000100368 | CSF2RB | colony stimulating factor 2 receptor beta | 0.01865112 |
| | | common subunit | |
| | | [Source: HGNC Symbol; Acc: HGNC: 2436] | |
| ENSG00000006015 | REX1BD | required for excision 1-B domain containing | 0.01871477 |
| | | [Source: HGNC Symbol; Acc: HGNC: 26098] | |
| ENSG00000011451 | WIZ | widely interspaced zinc finger motifs | 0.018812736 |
| | | [Source: HGNC Symbol; Acc: HGNC: 30917] | |
| ENSG00000160888 | IER2 | immediate early response 2 | 0.018812736 |
| | | [Source: HGNC Symbol; Acc: HGNC: 28871] | |
| ENSG00000174807 | CD248 | CD248 molecule | 0.018812736 |
| | | [Source: HGNC Symbol; Acc: HGNC: 18219] | |
| ENSG00000099821 | POLRMT | RNA polymerase mitochondrial | 0.018832922 |
| | | [Source: HGNC Symbol; Acc: HGNC: 9200] | |
| ENSG00000211899 | IGHM | immunoglobulin heavy constant mu | 0.018832922 |
| | | [Source: HGNC Symbol; Acc: HGNC: 5541] | |
| ENSG00000130313 | PGLS | 6-phosphogluconolactonase | 0.019015294 |
| | | [Source: HGNC Symbol; Acc: HGNC: 8903] | |
| ENSG00000165702 | GFI1B | growth factor independent 1B transcriptional | 0.019015294 |
| | | repressor | |
| | | [Source: HGNC Symbol; Acc: HGNC: 4238] | |
| ENSG00000196557 | CACNA1H | calcium voltage-gated channel subunit alpha1 H | 0.019108077 |
| | | [Source: HGNC Symbol; Acc: HGNC: 1395] | |
| ENSG00000188486 | H2AFX | H2A histone family member X | 0.019120708 |
| | | [Source: HGNC Symbol; Acc: HGNC: 4739] | |
| ENSG00000103260 | METRN | “meteorin, glial cell differentiation regulator | 0.01915234 |
| | | [Source: HGNC Symbol; Acc: HGNC: 14151]” | |
| ENSG00000166925 | TSC22D4 | TSC22 domain family member 4 | 0.01920241 |
| | | [Source: HGNC Symbol; Acc: HGNC: 21696] | |
| ENSG00000106266 | SNX8 | sorting nexin 8 | 0.019236897 |
| | | [Source: HGNC Symbol; Acc: HGNC: 14972] | |
| ENSG00000110400 | NECTIN1 | nectin cell adhesion molecule 1 | 0.01926502 |
| | | [Source: HGNC Symbol; Acc: HGNC: 9706] | |
| ENSG00000088992 | TESC | tescalcin | 0.019687862 |
| | | [Source: HGNC Symbol; Acc: HGNC: 26065] | |
| ENSG00000126368 | NR1D1 | nuclear receptor subfamily 1 group D member 1 | 0.019740714 |
| | | [Source: HGNC Symbol; Acc: HGNC: 7962] | |
| ENSG00000103202 | NME4 | NME/NM23 nucleoside diphosphate kinase 4 | 0.019829586 |
| | | [Source: HGNC Symbol: Acc: HGNC: 7852] | |
| ENSG00000213626 | LBH | limb bud and heart development | 0.019900152 |
| | | [Source: HGNC Symbol; Acc: HGNC: 29532] | |
| ENSG00000138629 | UBL7 | ubiquitin like 7 | 0.019916102 |
| | | [Source: HGNC Symbol; Acc: HGNC: 28221] | |
| ENSG00000254614 | | | 0.019916102 |
| ENSG00000116521 | SCAMP3s | ecretory carrier membrane protein 3 | 0.019953714 |
| | | [Source: HGNC Symbol: Acc: HGNC: 10565] | |
| ENSG00000132481 | TRIM47 | tripartite motif containing 47 | 0.019989295 |
| | | [Source: HGNC Symbol: Acc: HGNC: 19020] | |
| ENSG00000105699 | LSR | lipolysis stimulated lipoprotein receptor | 0.019999965 |
| | | [Source: HGNC Symbol: Acc: HGNC: 29572] | |
| ENSG00000125503 | PPP1R12C | protein phosphatase 1 regulatory subunit 12C | 0.020063533 |
| | | [Source: HGNC Symbol: Acc: HGNC: 14947] | |
| ENSG00000103056 | SMPD3 | sphingomyelin phosphodiesterase 3 | 0.020115844 |
| | | [Source: HGNC Symbol: Acc: HGNC: 14240] | |
| ENSG00000156381 | ANKRD9 | ankyrin repeat domain 9 | 0.020225168 |
| | | [Source: HGNC Symbol; Acc: HGNC: 20096] | |
| ENSG00000197471 | SPN | sialophorin | 0.020225168 |
| | | [Source: HGNC Symbol; Acc: HGNC: 11249] | |
| ENSG00000197471 | SPN | sialophorin | 0.020225168 |
| | | [Source: HGNC Symbol; Acc: HGNC: 11249] | |
| ENSG00000063854 | HAGH | hydroxyacylglutathione hydrolase | 0.020247513 |
| | | [Source: HGNC Symbol; Acc: HGNC: 4805] | |
| ENSG00000130590 | SAMD10 | sterile alpha motif domain containing 10 | 0.020258063 |
| | | [Source: HGNC Symbol; Acc: HGNC: 16129] | |
| ENSG00000167664 | TMIGD2 | transmembrane and immunoglobulin domain | 0.020258063 |
| | | containing 2 | |
| | | [Source: HGNC Symbol; Acc: HGNC: 28324] | |
| ENSG00000146083 | RNF44 | ring finger protein 44 | 0.020327471 |
| | | [Source: HGNC Symbol; Acc: HGNC: 19180] | |
| ENSG00000231925 | TAPBP | TAP binding protein | 0.020387859 |
| | | [Source: HGNC Symbol; Acc: HGNC: 11566] | |
| ENSG00000198858 | R3HDM4 | R3H domain containing 4 | 0.020462672 |
| | | [Source: HGNC Symbol; Acc: HGNC: 28270] | |
| ENSG00000135924 | DNAJB2 | DnaJ heat shock protein family (Hsp40) member B2 | 0.020505106 |
| | | [Source: HGNC Symbol; Acc: HGNC: 5228] | |
| ENSG00000239732 | TLR9 | toll like receptor 9 | 0.02065324 |
| | | [Source: HGNC Symbol; Acc: HGNC: 15633] | |
| ENSG00000115268 | RPS15 | ribosomal protein S15 | 0.020839375 |
| | | [Source: HGNC Symbol; Acc: HGNC: 10388] | |
| ENSG00000108798 | ABI3 | ABI family member 3 | 0.02085252 |
| | | [Source: HGNC Symbol; Acc: HGNC: 29859] | |
| ENSG00000119669 | IRF2BPL | interferon regulatory factor 2 binding protein like | 0.02099734 |
| | | [Source: HGNC Symbol; Acc: HGNC: 14282] | |
| ENSG00000160446 | ZDHHC12 | zinc finger DHHC-type containing 12 | 0.02150253 |
| | | [Source: HGNC Symbol; Acc: HGNC: 19159] | |
| ENSG00000063169 | BICRA | BRD4 interacting chromatin remodeling | 0.021525887 |
| | | complex associated protein | |
| | | [Source: HGNC Symbol; Acc: HGNC: 4332] | |
| ENSG00000141933 | TPGS1 | tubulin polyglutamylase complex subunit 1 | 0.021539966 |
| | | [Source: HGNC Symbol; Acc: HGNC: 25058] | |
| ENSG00000088256 | GNA11 | G protein subunit alpha 11 | 0.021557835 |
| | | [Source: HGNC Symbol; Acc: HGNC: 4379] | |
| ENSG00000169583 | CLIC3 | chloride intracellular channel 3 | 0.021557835 |
| | | [Source: HGNC Symbol; Acc: HGNC: 2064] | |
| ENSG00000188511 | C22orf34 | chromosome 22 open reading frame 34 | 0.021557835 |
| | | [Source: HGNC Symbol; Acc: HGNC: 28010] | |
| ENSG00000165406 | 8-Mar | membrane associated ring-CH-type finger 8 | 0.021577416 |
| | | [Source: HGNC Symbol; Acc: HGNC: 23356] | |
| ENSG00000173762 | CD7 | CD7 molecule | 0.021879276 |
| | | [Source: HGNC Symbol; Acc: HGNC: 1695] | |
| ENSG00000188322 | SBK1 | SH3 domain binding kinase 1 | 0.021879276 |
| | | [Source: HGNC Symbol: Acc: HGNC: 17699] | |
| ENSG00000204310 | AGPAT1 | 1-acylglycerol-3-phosphate O-acyltransferase 1 | 0.021879276 |
| | | [Source: HGNC Symbol: Acc: HGNC: 324] | |
| ENSG00000167797 | CDK2AP2 | cyclin dependent kinase 2 associated protein 2 | 0.021895705 |
| | | [Source: HGNC Symbol: Acc: HGNC: 30833] | |
| ENSG00000142669 | SH3BGRL3 | SH3 domain binding glutamate rich protein like 3 | 0.022007819 |
| | | [Source: HGNC Symbol: Acc: HGNC: 15568] | |
| ENSG00000155034 | FBXL18 | F-box and leucine rich repeat protein 18 | 0.022133956 |
| | | [Source: HGNC Symbol: Acc: HGNC: 21874] | |
| ENSG00000187840 | EIF4EBP1 | eukaryotic translation initiation factor 4E | 0.02227554 |
| | | binding protein 1 | |
| | | [Source: HGNC Symbol; Acc: HGNC: 3288] | |
| ENSG00000185187 | SIGIRR | single Ig and TIR domain containing | 0.022615882 |
| | | [Source: HGNC Symbol: Acc: HGNC: 30575] | |
| ENSG00000158545 | ZC3H18 | zinc finger CCCH-type containing 18 | 0.022649486 |
| | | [Source: HGNC Symbol; Acc: HGNC: 25091] | |
| ENSG00000184730 | APOBR | polipoprotein B receptor | 0.022689972 |
| | | [Source: HGNC Symbol; Acc: HGNC: 24087] | |
| ENSG00000204463 | BAG6 | BCL2 associated athanogene 6 | 0.022689972 |
| | | [Source: HGNC Symbol; Acc: HGNC: 13919] | |
| ENSG00000071242 | RPS6KA2 | ribosomal protein S6 kinase A2 | 0.022776502 |
| | | [Source: HGNC Symbol; Acc: HGNC: 10431] | |
| ENSG00000146701 | MDH2 | malate dehydrogenase 2 | 0.02289018 |
| | | [Source: HGNC Symbol; Acc: HGNC: 6971] | |
| ENSG00000180155 | LYNX1 | Ly6/neurotoxin 1 | 0.02289018 |
| | | [Source: HGNC Symbol; Acc: HGNC: 29604] | |
| ENSG00000213563 | C8orf82 | chromosome 8 open reading frame 82 | 0.023112388 |
| | | [Source: HGNC Symbol: Acc: HGNC: 33826] | |
| ENSG00000105281 | SLC1A5 | solute carrier family 1 member 5 | 0.023356543 |
| | | [Source: HGNC Symbol: Acc: HGNC: 10943] | |
| ENSG00000162882 | HAAO | “3-hydroxyanthranilate 3,4-dioxygenase | 0.02337834 |
| | | [Source: HGNC Symbol: Acc: HGNC: 4796]” | |
| ENSG00000181513 | ACBD4 | acyl-CoA binding domain containing 4 | 0.02337834 |
| | | [Source: HGNC Symbol: Acc: HGNC: 23337] | |
| ENSG00000185730 | ZNF696 | zinc finger protein 696 | 0.02337834 |
| | | [Source: HGNC Symbol: Acc: HGNC: 25872] | |
| ENSG00000007520 | TSR3 | “TSR3, acp transferase ribosome maturation | 0.023637109 |
| | | factor | |
| | | [Source: HGNC Symbol: Acc: HGNC: 14175]” | |
| ENSG00000090006 | LTBP4 | latent transforming growth factor beta binding | 0.023637109 |
| | | protein 4 | |
| | | [Source: HGNC Symbol: Acc: HGNC: 6717] | |
| ENSG00000146535 | GNA12 | G protein subunit alpha 12 | 0.023652058 |
| | | [Source: HGNC Symbol: Acc: HGNC: 4380] | |
| ENSG00000141965 | FEM1A | fem-1 homolog A | 0.023707215 |
| | | [Source: HGNC Symbol: Acc: HGNC: 16934] | |
| ENSG00000160957 | RECQL4 | RecQ like helicase 4 | 0.023710452 |
| | | [Source: HGNC Symbol; Acc: HGNC: 9949] | |
| ENSG00000135916 | ITM2C | integral membrane protein 2C | 0.023733416 |
| | | [Source: HGNC Symbol; Acc: HGNC: 6175] | |
| ENSG00000177732 | SOX12 | SRY-box 12 | 0.023733416 |
| | | [Source: HGNC Symbol; Acc: HGNC: 11198] | |
| ENSG00000184508 | HDDC3 | HD domain containing 3 | 0.023802652 |
| | | [Source: HGNC Symbol; Acc: HGNC: 30522] | |
| ENSG00000175591 | P2RY2 | purinergic receptor P2Y2 | 0.023918507 |
| | | [Source: HGNC Symbol; Acc: HGNC: 8541] | |
| ENSG00000127903 | ZNF835 | zinc finger protein 835 | 0.023926586 |
| | | [Source: HGNC Symbol; Acc: HGNC: 34332] | |
| ENSG00000176022 | B3GALT6 | “beta-1,3-galactosyltransferase 6 | 0.023926586 |
| | | [Source: HGNC Symbol; Acc: HGNC: 17978]” | |
| ENSG00000255319 | ENPP7P8 | ectonucleotide pyrophosphatase/phosphodiesterase | 0.02392953 |
| | | 7 pseudogene 8 | |
| | | [Source: HGNC Symbol; Acc: HGNC: 48691] | |
| ENSG00000105479 | CCDC114 | coiled-coil domain containing 114 | 0.02398767 |
| | | [Source: HGNC Symbol; Acc: HGNC: 26560] | |
| ENSG00000130529 | TRPM4 | transient receptor potential cation channel | 0.024020687 |
| | | subfamily M member 4 | |
| | | [Source: HGNC Symbol; Acc: HGNC: 17993] | |
| ENSG00000133250 | ZNF414 | zinc finger protein 414 | 0.024020687 |
| | | [Source: HGNC Symbol; Acc: HGNC: 20630] | |
| ENSG00000215908 | CROCCP2 | “ciliary rootlet coiled-coil, rootletin pseudogene 2 | 0.024052859 |
| | | [Source: HGNC Symbol; Acc: HGNC: 28170]” | |
| ENSG00000118046 | STK11 | serine/threonine kinase 11 | 0.024056107 |
| | | [Source: HGNC Symbol; Acc: HGNC: 11389] | |
| ENSG00000034152 | MAP2K3 | mitogen-activated protein kinase kinase 3 | 0.024155164 |
| | | [Source: HGNC Symbol; Acc: HGNC: 6843] | |
| ENSG00000142453 | CARM1 | coactivator associated arginine methyltransferase 1 | 0.024155164 |
| | | [Source: HGNC Symbol; Acc: HGNC: 23393] | |
| ENSG00000256323 | NA | NA | 0.024155164 |
| ENSG00000160094 | ZNF362 | zinc finger protein 362 | 0.024171066 |
| | | [Source: HGNC Symbol; Acc: HGNC: 18079] | |
| ENSG00000104884 | ERCC2 | “ERCC excision repair 2, TFIIH core complex | 0.024345083 |
| | | helicase subunit | |
| | | [Source: HGNC Symbol; Acc: HGNC: 3434]” | |
| ENSG00000149257 | SERPINH1 | serpin family H member 1 | 0.024345083 |
| | | [Source: HGNC Symbol; Acc: HGNC: 1546] | |
| ENSG00000169635 | HIC2 | HIC ZBTB transcriptional repressor 2 | 0.024354637 |
| | | [Source: HGNC Symbol; Acc: HGNC: 18595] | |
| ENSG00000143416 | SELENBP1 | selenium binding protein 1 | 0.024421599 |
| | | [Source: HGNC Symbol; Acc: HGNC: 10719] | |
| ENSG00000148411 | NACC2 | NACC family member 2 | 0.02443315 |
| | | [Source: HGNC Symbol; Acc: HGNC: 23846] | |
| ENSG00000085872 | CHERP | calcium homeostasis endoplasmic reticulum protein | 0.024463522 |
| | | [Source: HGNC Symbol; Acc: HGNC: 16930] | |
| ENSG00000176182 | MYPOP | “Myb related transcription factor, partner of profiling | 0.024477311 |
| | | [Source: HGNC Symbol; Acc: HGNC: 20178]” | |
| ENSG00000160113 | NR2F6 | nuclear receptor subfamily 2 group F member 6 | 0.024505177 |
| | | [Source: HGNC Symbol; Acc: HGNC: 7977] | |
| ENSG00000108262 | GIT1 | GIT ArfGAP 1 | 0.024614621 |
| | | [Source: HGNC Symbol; Acc: HGNC: 4272] | |
| ENSG00000161395 | PGAP3 | post-GPI attachment to proteins 3 | 0.024705126 |
| | | [Source: HGNC Symbol; Acc: HGNC: 23719] | |
| ENSG00000142089 | IFITM3 | interferon induced transmembrane protein 3 | 0.024765171 |
| | | [Source: HGNC Symbol; Acc: HGNC: 5414] | |
| ENSG00000070444 | MNT | MAX network transcriptional repressor | 0.025148395 |
| | | [Source: HGNC Symbol; Acc: HGNC: 7188] | |
| ENSG00000112514 | CUTA | cutA divalent cation tolerance homolog | 0.025148395 |
| | | [Source: HGNC Symbol; Acc: HGNC: 21101] | |
| ENSG00000167394 | ZNF668 | zinc finger protein 668 | 0.025148395 |
| | | [Source: HGNC Symbol; Acc: HGNC: 25821] | |
| ENSG00000167965 | MLST8 | “MTOR associated protein, LST8 homolog | 0.025148395 |
| | | [Source: HGNC Symbol; Acc: HGNC: 24825]” | |
| ENSG00000244187 | TMEM141 | transmembrane protein 141 | 0.025148395 |
| | | [Source: HGNC Symbol; Acc: HGNC: 28211] | |
| ENSG00000218175 | | | 0.02527518 |
| ENSG00000110063 | DCPS | “decapping enzyme, scavenger | 0.025300524 |
| | | [Source: HGNC Symbol; Acc: HGNC: 29812]” | |
| ENSG00000128805 | ARHGAP22 | Rho GTPase activating protein 22 | 0.025318552 |
| | | [Source: HGNC Symbol; Acc: HGNC: 30320] | |
| ENSG00000148400 | NOTCH1 | notch 1 | 0.025506427 |
| | | [Source: HGNC Symbol; Acc; HGNC: 7881] | |
| ENSG00000186076 | | | 0.0257616 |
| ENSG00000167470 | MIDN | midnolin | 0.02580767 |
| | | [Source: HGNC Symbol; Acc: HGNC: 16298] | |
| ENSG00000188305 | PEAK3 | PEAK family member 3 | 0.02580767 |
| | | [Source: HGNC Symbol; Acc: HGNC: 24793] | |
| ENSG00000181396 | OGFOD3 | 2-oxoglutarate and iron dependent oxygenase | 0.026198035 |
| | | domain containing 3 | |
| | | [Source: HGNC Symbol; Acc: HGNC: 26174] | |
| ENSG00000240877 | RN7SL521P | “RNA, 7SL, cytoplasmic 521, pseudogene | 0.026492508 |
| | | [Source: HGNC Symbol; Acc: HGNC: 46537]” | |
| ENSG00000130511 | SSBP4 | single stranded DNA binding protein 4 | 0.026588317 |
| | | [Source: HGNC Symbol; Acc: HGNC: 15676] | |
| ENSG00000063177 | RPL18 | ribosomal protein L18 | 0.026698079 |
| | | [Source: HGNC Symbol; Acc: HGNC: 10310] | |
| ENSG00000172663 | TMEM134 | transmembrane protein 134 | 0.026698079 |
| | | [Source: HGNC Symbol; Acc: HGNC: 26142] | |
| ENSG00000130706 | ADRM1 | adhesion regulating molecule 1 | 0.026721536 |
| | | [Source: HGNC Symbol; Acc: HGNC: 15759] | |
| ENSG00000214063 | TSPAN4 | tetraspanin 4 | 0.026759131 |
| | | [Source: HGNC Symbol; Acc: HGNC: 11859] | |
| ENSG00000161677 | JOSD2 | Josephin domain containing 2 | 0.026798474 |
| | | [Source: HGNC Symbol; Acc: HGNC: 28853] | |
| ENSG00000189060 | H1F0 | H1 histone family member 0 | 0.027109095 |
| | | [Source: HGNC Symbol; Acc: HGNC: 4714] | |
| ENSG00000256811 | | | 0.027109095 |
| ENSG00000133317 | LGALS12 | galectin 12 | 0.027164347 |
| | | [Source: HGNC Symbol; Acc: HGNC: 15788] | |
| ENSG00000012061 | ERCC1 | “ERCC excision repair 1, endonuclease | 0.027196959 |
| | | non-catalytic subunit | |
| | | [Source: HGNC Symbol; Acc: HGNC: 3433]” | |
| ENSG00000007376 | RPUSD1 | RNA pseudouridylate synthase domain containing 1 | 0.027275974 |
| | | [Source: HGNC Symbol; Acc: HGNC: 14173] | |
| ENSG00000108175 | ZMIZ1 | zinc finger MIZ-type containing 1 | 0.027331142 |
| | | [Source: HGNC Symbol; Acc: HGNC: 16493] | |
| ENSG00000132003 | ZSWIM4 | zinc finger SWIM-type containing 4 | 0.027331142 |
| | | [Source: HGNC Symbol; Acc: HGNC: 25704] | |
| ENSG00000148296 | SURF6 | surfeit 6 | 0.027362837 |
| | | [Source: HGNC Symbol; Acc: HGNC: 11478] | |
| ENSG00000186056 | MATN1-AS1 | MATN1 antisense RNA 1 | 0.02742002 |
| | | [Source: HGNC Symbol; Acc: HGNC: 40364] | |
| ENSG00000115649 | CNPPD1 | cyclin Pas1/PHO80 domain containing 1 | 0.027574972 |
| | | [Source: HGNC Symbol; Acc: HGNC: 25220] | |
| ENSG00000065057 | NTHL1 | nth like DNA glycosylase 1 | 0.027763734 |
| | | [Source: HGNC Symbol; Acc: HGNC: 8028] | |
| ENSG00000272098 | NA | NA | 0.027825114 |
| ENSG00000011009 | LYPLA2 | lysophospholipase II | 0.028071412 |
| | | [Source: HGNC Symbol; Acc: HGNC: 6738] | |
| ENSG00000110025 | SNX15 | sorting nexin 15 | 0.028074538 |
| | | [Source: HGNC Symbol; Acc: HGNC: 14978] | |
| ENSG00000095321 | CRAT | carnitine O-acetyltransferase | 0.028133383 |
| | | [Source: HGNC Symbol; Acc: HGNC: 2342] | |
| ENSG00000108515 | ENO3 | enolase 3 | 0.028133383 |
| | | [Source: HGNC Symbol; Acc: HGNC: 3354] | |
| ENSG00000123064 | DDX54 | DEAD-box helicase 54 | 0.028358899 |
| | | [Source: HGNC Symbol; Acc: HGNC: 20084] | |
| ENSG00000169564 | PCBP1 | poly(rC) binding protein 1 | 0.028645846 |
| | | [Source: HGNC Symbol; Acc: HGNC: 8647] | |
| ENSG00000171045 | TSNARE1 | t-SNARE domain containing 1 | 0.028645846 |
| | | [Source: HGNC Symbol; Acc: HGNC: 26437] | |
| ENSG00000225978 | HAR1A | highly accelerated region 1A (non-protein coding) | 0.028645846 |
| | | [Source: HGNC Symbol; Acc: HGNC: 33117] | |
| ENSG00000128283 | CDC42EP1 | CDC42 effector protein 1 | 0.028675863 |
| | | [Source: HGNC Symbol; Acc: HGNC: 17014] | |
| ENSG00000174282 | ZBTB4 | zinc finger and BTB domain containing 4 | 0.028871519 |
| | | [Source: HGNC Symbol; Acc: HGNC: 23847] | |
| ENSG00000167685 | ZNF444 | zinc finger protein 444 | 0.028919683 |
| | | [Source: HGNC Symbol; Acc: HGNC: 16052] | |
| ENSG00000110104 | CCDC86 | coiled-coil domain containing 86 | 0.028929404 |
| | | [Source: HGNC Symbol; Acc: HGNC: 28359] | |
| ENSG00000171703 | TCEA2 | transcription elongation factor A2 | 0.029117009 |
| | | [Source: HGNC Symbol; Acc: HGNC: 11614] | |
| ENSG00000177600 | RPLP2 | ribosomal protein lateral stalk subunit P2 | 0.029298722 |
| | | [Source: HGNC Symbol; Acc: HGNC: 10377] | |
| ENSG00000182095 | TNRC18 | trinucleotide repeat containing 18 | 0.029299181 |
| | | [Source: HGNC Symbol; Acc: HGNC: 11962] | |
| ENSG00000167106 | FAM102A | family with sequence similarity 102 member A | 0.029415219 |
| | | [Source: HGNC Symbol; Acc: HGNC: 31419] | |
| ENSG00000126458 | RRAS | RAS related | 0.02952737 |
| | | [Source: HGNC Symbol; Acc: HGNC: 10447] | |
| ENSG00000105063 | PPP6R1 | protein phosphatase 6 regulatory subunit 1 | 0.02959944 |
| | | [Source: HGNC Symbol; Acc: HGNC: 29195] | |
| ENSG00000125730 | C3 | complement C3 | 0.029997906 |
| | | [Source: HGNC Symbol; Acc: HGNC: 1318] | |
| ENSG00000237973 | MTCO1P12 | mitochondrially encoded cytochrome c oxidase | 0.030144659 |
| | | I pseudogene 12 | |
| | | [Source: HGNC Symbol; Acc: HGNC: 52014] | |
| ENSG00000267412 | | | 0.030144659 |
| ENSG00000185813 | PCYT2 | “phosphate cytidylyltransferase 2, ethanolamine | 0.030317293 |
| | | [Source: HGNC Symbol; Acc: HGNC: 8756]” | |
| ENSG00000163462 | TRIM46 | tripartite motif containing 46 | 0.030614393 |
| | | [Source: HGNC Symbol; Acc: HGNC: 19019] | |
| ENSG00000157933 | SKI | SKI proto-oncogene | 0.030701411 |
| | | [Source: HGNC Symbol; Acc: HGNC: 10896] | |
| ENSG00000161091 | MFSD12 | major facilitator superfamily domain containing 12 | 0.030704292 |
| | | [Source: HGNC Symbol; Acc: HGNC: 28299] | |
| ENSG00000185163 | DDX51 | DEAD-box helicase 51 | 0.030735268 |
| | | [Source: HGNC Symbol; Acc: HGNC: 20082] | |
| ENSG00000171813 | PWWP2B | PWWP domain containing 2B | 0.030810478 |
| | | [Source: HGNC Symbol; Acc: HGNC: 25150] | |
| ENSG00000137267 | TUBB2A | tubulin beta 2A class IIa | 0.030847037 |
| | | [Source: HGNC Symbol; Acc: HGNC: 12412] | |
| ENSG00000188747 | NOXA1 | NADPH oxidase activator 1 | 0.030853611 |
| | | [Source: HGNC Symbol; Acc: HGNC: 10668] | |
| ENSG00000108557 | RAI1 | retinoic acid induced 1 | 0.030977697 |
| | | [Source: HGNC Symbol; Acc: HGNC: 9834] | |
| ENSG00000137166 | FOXP4 | forkhead box P4 | 0.030977697 |
| | | [Source: HGNC Symbol; Acc: HGNC: 20842] | |
| ENSG00000204420 | MPIG6B | megakaryocyte and platelet inhibitory receptor G6b | 0.031369168 |
| | | [Source: HGNC Symbol; Acc: HGNC: 13937] | |
| ENSG00000133265 | HSPBP1 | HSPA (Hsp70) binding protein 1 | 0.031442624 |
| | | [Source: HGNC Symbol; Acc: HGNC: 24989] | |
| ENSG00000008710 | PKD1 | “polycystin 1, transient receptor potential | 0.031566582 |
| | | channel interacting | |
| | | [Source: HGNC Symbol; Acc: HGNC: 9008]” | |
| ENSG00000099624 | ATP5F1D | ATP synthase F1 subunit delta | 0.031657927 |
| | | [Source: HGNC Symbol; Acc: HGNC: 837] | |
| ENSG00000108819 | PPP1R9B | protein phosphatase 1 regulatory subunit 9B | 0.031695534 |
| | | [Source: HGNC Symbol; Acc: HGNC: 9298] | |
| ENSG00000158292 | GPR153 | G protein-coupled receptor 153 | 0.031750316 |
| | | [Source: HGNC Symbol; Acc: HGNC: 23618] | |
| ENSG00000130382 | MLLT1 | “MLLT1, super elongation complex subunit | 0.031828022 |
| | | [Source: HGNC Symbol; Acc: HGNC: 7134]” | |
| ENSG00000269352 | PTOV1-AS2 | PTOV1 antisense RNA 2 | 0.031850457 |
| | | [Source: HGNC Symbol; Acc: HGNC: 51284] | |
| ENSG00000162585 | FAAP20 | Fanconi anemia core complex associated protein 20 | 0.0320507 |
| | | [Source: HGNC Symbol; Acc: HGNC: 26428] | |
| ENSG00000157240 | FZD1 | frizzled class receptor 1 | 0.032151732 |
| | | [Source: HGNC Symbol; Acc: HGNC: 4038] | |
| ENSG00000135736 | CCDC102A | coiled-coil domain containing 102A | 0.032302006 |
| | | [Source: HGNC Symbol; Acc: HGNC: 28097] | |
| ENSG00000020181 | ADGRA2 | adhesion G protein-coupled receptor A2 | 0.032424937 |
| | | [Source: HGNC Symbol; Acc: HGNC: 17849] | |
| ENSG00000198546 | ZNF511 | zinc finger protein 511 | 0.032576231 |
| | | [Source: HGNC Symbol; Acc: HGNC: 28445] | |
| ENSG00000123144 | TRIR | telomerase RNA component interacting RNase | 0.032671184 |
| | | [Source: HGNC Symbol; Acc: HGNC: 28424] | |
| ENSG00000156860 | FBRS | fibrosin | 0.032671184 |
| | | [Source: HGNC Symbol; Acc: HGNC: 20442] | |
| ENSG00000162910 | MRPL55 | mitochondrial ribosomal protein L55 | 0.032697662 |
| | | [Source: HGNC Symbol; Acc: HGNC: 16686] | |
| ENSG00000130731 | METTL26 | methyltransferase like 26 | 0.032833764 |
| | | [Source: HGNC Symbol; Acc: HGNC: 14141] | |
| ENSG00000101986 | ABCD1 | ATP binding cassette subfamily D member 1 | 0.032886018 |
| | | [Source: HGNC Symbol; Acc: HGNC: 61] | |
| ENSG00000020633 | RUNX3 | runt related transcription factor 3 | 0.033016587 |
| | | [Source: HGNC Symbol; Acc: HGNC: 10473] | |
| ENSG00000184640 | 9-Sep | septin 9 | 0.033101928 |
| | | [Source: HGNC Symbol; Acc: HGNC: 7323] | |
| ENSG00000260521 | NA | NA | 0.033101928 |
| ENSG00000125787 | GNRH2 | gonadotropin releasing hormone 2 | 0.033349502 |
| | | [Source: HGNC Symbol; Acc: HGNC: 4420] | |
| ENSG00000229391 | HLA-DRB6 | “major histocompatibility complex, class II, | 0.033349502 |
| | | DR beta 6 (pseudogene) | |
| | | [Source: HGNC Symbol; Acc: HGNC: 4954]” | |
| ENSG00000160223 | ICOSLG | inducible T cell costimulator ligand | 0.033394348 |
| | | [Source: HGNC Symbol; Acc: HGNC: 17087] | |
| ENSG00000105204 | DYRK1B | dual specificity tyrosine phosphorylation | 0.033465567 |
| | | regulated kinase 1B | |
| | | [Source: HGNC Symbol; Acc: HGNC: 3092] | |
| ENSG00000142173 | COL6A2 | collagen type VI alpha 2 chain | 0.033483825 |
| | | [Source: HGNC Symbol; Acc: HGNC: 2212] | |
| ENSG00000169710 | FASN | fatty acid synthase | 0.033483825 |
| | | [Source: HGNC Symbol; Acc: HGNC: 3594] | |
| ENSG00000176533 | GNG7 | G protein subunit gamma 7 | 0.033483825 |
| | | [Source: HGNC Symbol; Acc: HGNC: 4410] | |
| ENSG00000179253 | | | 0.033483825 |
| ENSG00000169972 | PUSL1 | pseudouridylate synthase-like 1 | 0.033509544 |
| | | [Source: HGNC Symbol; Acc: HGNC: 26914] | |
| ENSG00000160360 | GPSM1 | G protein signaling modulator 1 | 0.033771567 |
| | | [Source: HGNC Symbol; Acc: HGNC: 17858] | |
| ENSG00000171159 | C9orf16 | chromosome 9 open reading frame 16 | 0.033853634 |
| | | [Source: HGNC Symbol; Acc: HGNC: 17823] | |
| ENSG00000215375 | MYL5 | myosin light chain 5 | 0.033853634 |
| | | [Source: HGNC Symbol; Acc: HGNC: 7586] | |
| ENSG00000105402 | NAPA | NSF attachment protein alpha | 0.034044441 |
| | | [Source: HGNC Symbol; Acc: HGNC: 7641] | |
| ENSG00000038532 | CLEC16A | C-type lectin domain containing 16A | 0.034108157 |
| | | [Source: HGNC Symbol; Acc: HGNC: 29013] | |
| ENSG00000165175 | MID1IP1 | MID1 interacting protein 1 | 0.03418982 |
| | | [Source: HGNC Symbol; Acc: HGNC: 20715] | |
| ENSG00000166947 | EPB42 | erythrocyte membrane protein band 4.2 | 0.03418982 |
| | | [Source: HGNC Symbol; Acc: HGNC: 3381] | |
| ENSG00000168286 | THAP11 | THAP domain containing 11 | 0.03418982 |
| | | [Source: HGNC Symbol; Acc: HGNC: 23194] | |
| ENSG00000168476 | REEP4 | receptor accessory protein 4 | 0.034591261 |
| | | [Source: HGNC Symbol; Acc: HGNC: 26176] | |
| ENSG00000107521 | HPS1 | “HPS1, biogenesis of lysosomal organelles | 0.034688533 |
| | | complex 3 subunit 1 | |
| | | [Source: HGNC Symbol; Acc: HGNC: 5163]” | |
| ENSG00000267436 | | | 0.034821851 |
| ENSG00000099991 | CABIN1 | calcineurin binding protein 1 | 0.034858474 |
| | | [Source: HGNC Symbol; Acc: HGNC: 24187] | |
| ENSG00000169718 | DUS1L | dihydrouridine synthase 1 like | 0.034980702 |
| | | [Source: HGNC Symbol; Acc: HGNC: 30086] | |
| ENSG00000105325 | FZR1 | fizzy and cell division cycle 20 related 1 | 0.035034499 |
| | | [Source: HGNC Symbol; Acc: HGNC: 24824] | |
| ENSG00000167291 | TBC1D16 | TBC1 domain family member 16 | 0.035082388 |
| | | [Source: HGNC Symbol; Acc: HGNC: 28356] | |
| ENSG00000213399 | | | 0.035085538 |
| ENSG00000175040 | CHST2 | carbohydrate sulfotransferase 2 | 0.035106231 |
| | | [Source: HGNC Symbol; Acc: HGNC: 1970] | |
| ENSG00000228544 | CCDC183-AS1 | CCDC183 antisense RNA 1 | 0.035154264 |
| | | [Source: HGNC Symbol; Acc: HGNC: 44105] | |
| ENSG00000167658 | EEF2 | eukaryotic translation elongation factor 2 | 0.03521308 |
| | | [Source: HGNC Symbol; Acc: HGNC: 3214] | |
| ENSG00000090238 | YPEL3 | yippee like 3 | 0.035223199 |
| | | [Source: HGNC Symbol; Acc: HGNC: 18327] | |
| ENSG00000172508 | CARNS1 | carnosine synthase 1 | 0.03555645 |
| | | [Source: HGNC Symbol; Acc: HGNC: 29268] | |
| ENSG00000173272 | MZT2A | mitotic spindle organizing protein 2A | 0.035664739 |
| | | [Source: HGNC Symbol; Acc: HGNC: 33187] | |
| ENSG00000141522 | ARHGDIA | Rho GDP dissociation inhibitor alpha | 0.03575577 |
| | | [Source: HGNC Symbol; Acc: HGNC: 678] | |
| ENSG00000149541 | B3GAT3 | “beta-1,3-glucuronyltransferase 3 | 0.03575577 |
| | | [Source: HGNC Symbol; Acc: HGNC: 923]” | |
| ENSG00000171206 | TRIM8 | tripartite motif containing 8 | 0.035790036 |
| | | [Source: HGNC Symbol; Acc: HGNC: 15579] | |
| ENSG00000027869 | SH2D2A | SH2 domain containing 2A | 0.035871776 |
| | | [Source: HGNC Symbol; Acc: HGNC: 10821] | |
| ENSG00000149823 | VPS51 | “VPS51, GARP complex subunit | 0.035906645 |
| | | [Source: HGNC Symbol; Acc: HGNC: 1172]” | |
| ENSG00000196355 | NA | NA | 0.035938935 |
| ENSG00000165804 | ZNF219 | zinc finger protein 219 | 0.035996581 |
| | | [Source: HGNC Symbol; Acc: HGNC: 13011] | |
| ENSG00000177542 | SLC25A22 | solute carrier family 25 member 22 | 0.035996581 |
| | | [Source: HGNC Symbol; Acc: HGNC: 19954] | |
| ENSG00000130202 | NECTIN2 | nectin cell adhesion molecule 2 | 0.036025366 |
| | | [Source: HGNC Symbol; Acc: HGNC: 9707] | |
| ENSG00000006638 | TBXA2R | thromboxane A2 receptor | 0.036065767 |
| | | [Source: HGNC Symbol; Acc: HGNC: 11608] | |
| ENSG00000025770 | NCAPH2 | non-SMC condensin II complex subunit H2 | 0.036065767 |
| | | [Source: HGNC Symbol; Acc: HGNC: 25071] | |
| ENSG00000100316 | RPL3 | ribosomal protein L3 | 0.036065767 |
| | | [Source: HGNC Symbol; Acc: HGNC: 10332] | |
| ENSG00000110665 | C11orf21 | chromosome 11 open reading frame 21 | 0.036065767 |
| | | [Source: HGNC Symbol; Acc: HGNC: 13231] | |
| ENSG00000160445 | ZER1 | zyg-11 related cell cycle regulator | 0.036065767 |
| | | [Source: HGNC Symbol; Acc: HGNC: 30960] | |
| ENSG00000173786 | CNP | “2′,3′-cyclic nucleotide 3′ phosphodiesterase | 0.036065767 |
| | | [Source: HGNC Symbol; Acc: HGNC: 2158]” | |
| ENSG00000229368 | | | 0.036065767 |
| ENSG00000160789 | LMNA | lamin A/C | 0.036188381 |
| | | [Source: HGNC Symbol; Acc: HGNC: 6636] | |
| ENSG00000166189 | HPS6 | “HPS6, biogenesis of lysosomal organelles | 0.036188381 |
| | | complex 2 subunit 3 | |
| | | [Source: HGNC Symbol: Acc: HGNC: 18817]” | |
| ENSG00000261226 | | | 0.036342968 |
| ENSG00000185049 | NELFA | negative elongation factor complex member A | 0.036383164 |
| | | [Source: HGNC Symbol: Acc: HGNC: 12768] | |
| ENSG00000005882 | PDK2 | pyruvate dehydrogenase kinase 2 | 0.036423123 |
| | | [Source: HGNC Symbol: Acc: HGNC: 8810] | |
| ENSG00000163050 | COQ8A | coenzyme Q8A | 0.036459828 |
| | | [Source: HGNC Symbol: Acc: HGNC: 16812] | |
| ENSG00000060138 | YBX3 | Y-box binding protein 3 | 0.03651176 |
| | | [Source: HGNC Symbol: Acc: HGNC: 2428] | |
| ENSG00000122971 | ACADS | acyl-CoA dehydrogenase short chain | 0.036511776 |
| | | [Source: HGNC Symbol: Acc: HGNC: 90] | |
| ENSG00000205927 | OLIG2 | oligodendrocyte transcription factor 2 | 0.036582868 |
| | | [Source: HGNC Symbol; Acc: HGNC: 9398] | |
| ENSG00000092096 | SLC22A17 | solute carrier family 22 member 17 | 0.036619295 |
| | | [Source: HGNC Symbol; Acc: HGNC: 23095] | |
| ENSG00000090554 | FLT3LG | fms related tyrosine kinase 3 ligand | 0.036780281 |
| | | [Source: HGNC Symbol; Acc: HGNC: 3766] | |
| ENSG00000078902 | TOLLIP | toll interacting protein | 0.036796695 |
| | | [Source: HGNC Symbol; Acc: HGNC: 16476] | |
| ENSG00000136802 | LRRC8A | leucine rich repeat containing 8 VRAC subunit A | 0.036860551 |
| | | [Source: HGNC Symbol; Acc: HGNC: 19027] | |
| ENSG00000236976 | | | 0.036972133 |
| ENSG00000100908 | EMC9 | ER membrane protein complex subunit 9 | 0.037054085 |
| | | [Source: HGNC Symbol; Acc: HGNC: 20273] | |
| ENSG00000105364 | MRPL4 | mitochondrial ribosomal protein L4 | 0.03712252 |
| | | [Source: HGNC Symbol; Acc: HGNC: 14276] | |
| ENSG00000157184 | CPT2 | carnitine palmitoyltransferase 2 | 0.03712252 |
| | | [Source: HGNC Symbol; Acc: HGNC: 2330] | |
| ENSG00000168056 | LTBP3 | latent transforming growth factor beta binding | 0.037135417 |
| | | protein 3 | |
| | | [Source: HGNC Symbol; Acc: HGNC: 6716] | |
| ENSG00000196313 | POM121 | POM121 transmembrane nucleoporin | 0.037140956 |
| | | [Source: HGNC Symbol; Acc: HGNC: 19702] | |
| ENSG00000170604 | IRF2BP1 | nterferon regulatory factor 2 binding protein 1 | 0.037222007 |
| | | [Source: HGNC Symbol; Acc: HGNC: 21728] | |
| ENSG00000110697 | PITPNM1 | phosphatidylinositol transfer protein membrane | 0.037374978 |
| | | associated 1 | |
| | | [Source: HGNC Symbol; Acc: HGNC: 9003] | |
| ENSG00000100348 | TXN2 | thioredoxin 2 | 0.037546969 |
| | | [Source: HGNC Symbol; Acc: HGNC: 17772] | |
| ENSG00000102007 | PLP2 | proteolipid protein 2 | 0.037630556 |
| | | [Source: HGNC Symbol: Acc: HGNC: 9087] | |
| ENSG00000132005 | RFX1 | regulatory factor X1 | 0.037671165 |
| | | [Source: HGNC Symbol: Acc: HGNC: 9982] | |
| ENSG00000141499 | WRAP53 | WD repeat containing antisense to TP53 | 0.037678229 |
| | | [Source: HGNC Symbol; Acc: HGNC: 25522] | |
| ENSG00000189114 | BLOC1S3 | biogenesis of lysosomal organelles complex 1 | 0.037963219 |
| | | subunit 3 | |
| | | [Source: HGNC Symbol; Acc: HGNC: 20914] | |
| ENSG00000123154 | WDR83 | WD repeat domain 83 | 0.038059131 |
| | | [Source: HGNC Symbol; Acc: HGNC: 32672] | |
| ENSG00000127663 | KDM4B | lysine demethylase 4B | 0.038059131 |
| | | [Source: HGNC Symbol; Acc: HGNC: 29136] | |
| ENSG00000175274 | TP53I11 | tumor protein p53 inducible protein 11 | 0.038059131 |
| | | [Source: HGNC Symbol; Acc: HGNC: 16842] | |
| ENSG00000249115 | HAUS5 | HAUS augmin like complex subunit 5 | 0.038059131 |
| | | [Source: HGNC Symbol; Acc: HGNC: 29130] | |
| ENSG00000130764 | LRRC47 | leucine rich repeat containing 47 | 0.038286929 |
| | | [Source: HGNC Symbol; Acc: HGNC: 29207] | |
| ENSG00000176946 | THAP4 | THAP domain containing 4 | 0.038286929 |
| | | [Source: HGNC Symbol; Acc: HGNC: 23187] | |
| ENSG00000137497 | NUMA1 | nuclear mitotic apparatus protein 1 | 0.038678067 |
| | | [Source: HGNC Symbol; Acc: HGNC: 8059] | |
| ENSG00000143761 | ARF1 | ADP ribosylation factor 1 | 0.038678067 |
| | | [Source: HGNC Symbol; Acc: HGNC: 652] | |
| ENSG00000198931 | APRT | adenine phosphoribosyltransferase | 0.038678067 |
| | | [Source: HGNC Symbol; Acc: HGNC: 626] | |
| ENSG00000186174 | BCL9L | B cell CLL/lymphoma 9 like | 0.039375488 |
| | | [Source: HGNC Symbol; Acc: HGNC: 23688] | |
| ENSG00000104894 | CD37 | CD37 molecule | 0.039523734 |
| | | [Source: HGNC Symbol; Acc: HGNC: 1666] | |
| ENSG00000235314 | LINC00957 | long intergenic non-protein coding RNA 957 | 0.039534059 |
| | | [Source: HGNC Symbol; Acc: HGNC: 22332] | |
| ENSG00000076864 | RAP1GAP | RAP1 GTPase activating protein | 0.039534171 |
| | | [Source: HGNC Symbol; Acc: HGNC: 9858] | |
| ENSG00000179348 | GATA2 | GATA binding protein 2 | 0.039644975 |
| | | [Source: HGNC Symbol; Acc: HGNC: 4171] | |
| ENSG00000223496 | EXOSC6 | exosome component 6 | 0.039646513 |
| | | [Source: HGNC Symbol; Acc: HGNC: 19055] | |
| ENSG00000174004 | NRROS | negative regulator of reactive oxygen species | 0.039697731 |
| | | [Source: HGNC Symbol; Acc: HGNC: 24613] | |
| ENSG00000185736 | ADARB2 | “adenosine deaminase, RNA specific B2 (inactive) | 0.039813584 |
| | | [Source: HGNC Symbol; Acc: HGNC: 227]” | |
| ENSG00000177595 | PIDD1 | p53-induced death domain protein 1 | 0.039841437 |
| | | [Source: HGNC Symbol; Acc: HGNC: 16491] | |
| ENSG00000114767 | RRP9 | “ribosomal RNA processing 9, U3 small | 0.039852243 |
| | | nucleolar RNA binding protein | |
| | | [Source: HGNC Symbol; Acc: HGNC: 16829]” | |
| ENSG00000198336 | MYL4 | myosin light chain 4 | 0.040061329 |
| | | [Source: HGNC Symbol; Acc: HGNC: 7585] | |
| ENSG00000267427 | NA | NA | 0.040061329 |
| ENSG00000123159 | GIPC1 | GIPC PDZ domain containing family member 1 | 0.040207239 |
| | | [Source: HGNC Symbol; Acc: HGNC: 1226] | |
| ENSG00000139405 | RITA1 | RBPJ interacting and tubulin associated 1 | 0.040571361 |
| | | [Source: HGNC Symbol; Acc: HGNC: 25925] | |
| ENSG00000149929 | HIRIP3 | HIRA interacting protein 3 | 0.040590193 |
| | | [Source: HGNC Symbol; Acc: HGNC: 4917] | |
| ENSG00000198517 | MAFK | MAF bZIP transcription factor K | 0.040590193 |
| | | [Source: HGNC Symbol; Acc: HGNC: 6782] | |
| ENSG00000164897 | TMUB1 | transmembrane and ubiquitin like domain | 0.040938395 |
| | | containing 1 | |
| | | [Source: HGNC Symbol; Acc: HGNC: 21709] | |
| ENSG00000070047 | PHRF1 | PHD and ring finger domains 1 | 0.041015111 |
| | | [Source: HGNC Symbol; Acc: HGNC: 24351] | |
| ENSG00000100403 | ZC3H7B | zinc finger CCCH-type containing 7B | 0.041151359 |
| | | [Source: HGNC Symbol; Acc: HGNC: 30869] | |
| ENSG00000205147 | NA | NA | 0.041207854 |
| ENSG00000184470 | TXNRD2 | thioredoxin reductase 2 | 0.04134465 |
| | | [Source: HGNC Symbol; Acc: HGNC: 18155] | |
| ENSG00000103145 | HCFC1R1 | host cell factor C1 regulator 1 | 0.041366672 |
| | | [Source: HGNC Symbol; Acc: HGNC: 21198] | |
| ENSG00000087086 | FTL | ferritin light chain | 0.041631474 |
| | | [Source: HGNC Symbol; Acc: HGNC: 3999] | |
| ENSG00000102870 | ZNF629 | zinc finger protein 629 | 0.041631474 |
| | | [Source: HGNC Symbol; Acc: HGNC: 29008] | |
| ENSG00000181444 | ZNF467 | zinc finger protein 467 | 0.041868067 |
| | | [Source: HGNC Symbol; Acc: HGNC: 23154] | |
| ENSG00000142444 | TIMM29 | translocase of inner mitochondrial membrane 29 | 0.041933219 |
| | | [Source: HGNC Symbol; Acc: HGNC: 25152] | |
| ENSG00000204252 | HLA-DOA | “major histocompatibility complex, class II, DO alpha | 0.041944166 |
| | | [Source: HGNC Symbol; Acc: HGNC: 4936]” | |
| ENSG00000224051 | CPTP | ceramide-1-phosphate transfer protein | 0.041944166 |
| | | [Source: HGNC Symbol; Acc: HGNC: 28116] | |
| ENSG00000103253 | HAGHL | hydroxyacylglutathione hydrolase like | 0.042027801 |
| | | [Source: HGNC Symbol; Acc: HGNC: 14177] | |
| ENSG00000011590 | ZBTB32 | zinc finger and BTB domain containing 32 | 0.042101327 |
| | | [Source: HGNC Symbol; Acc: HGNC: 16763] | |
| ENSG00000182566 | CLEC4G | C-type lectin domain family 4 member G | 0.042114834 |
| | | [Source: HGNC Symbol; Acc: HGNC: 24591] | |
| ENSG00000244165 | P2RY11 | purinergic receptor P2Y11 | 0.042114834 |
| | | [Source: HGNC Symbol; Acc: HGNC: 8540] | |
| ENSG00000267275 | | | 0.042114834 |
| ENSG00000173327 | MAP3K11 | mitogen-activated protein kinase kinase kinase 11 | 0.042395519 |
| | | [Source: HGNC Symbol; Acc: HGNC: 6850] | |
| ENSG00000149418 | ST14 | suppression of tumorigenicity 14 | 0.04243773 |
| | | [Source: HGNC Symbol; Acc: HGNC: 11344] | |
| ENSG00000112658 | SRF | serum response factor | 0.042440717 |
| | | [Source: HGNC Symbol; Acc: HGNC: 11291] | |
| ENSG00000106003 | LFNG | LFNG O-fucosylpeptide | 0.042450091 |
| | | 3-beta-N-acetylglucosaminyltransferase | |
| | | [Source: HGNC Symbol; Acc: HGNC: 6560] | |
| ENSG00000164896 | FASTK | Fas activated serine/threonine kinase | 0.042450091 |
| | | [Source: HGNC Symbol; Acc: HGNC: 24676] | |
| ENSG00000196544 | BORCS6 | BLOC-1 related complex subunit 6 | 0.042514351 |
| | | [Source: HGNC Symbol; Acc: HGNC: 25939] | |
| ENSG00000134107 | BHLHE40 | basic helix-loop-helix family member e40 | 0.042517218 |
| | | [Source: HGNC Symbol; Acc: HGNC: 1046] | |
| ENSG00000151176 | PLBD2 | phospholipase B domain containing 2 | 0.042582368 |
| | | [Source: HGNC Symbol; Acc: HGNC: 27283] | |
| ENSG00000183397 | C19orf71 | chromosome 19 open reading frame 71 | 0.042902315 |
| | | [Source: HGNC Symbol; Acc: HGNC: 34496] | |
| ENSG00000105738 | SIPA1L3 | signal induced proliferation associated 1 like 3 | 0.043052047 |
| | | [Source: HGNC Symbol; Acc: HGNC: 23801] | |
| ENSG00000157353 | FUK | fucokinase | 0.043381308 |
| | | [Source: HGNC Symbol; Acc: HGNC: 29500] | |
| ENSG00000126062 | TMEM115 | transmembrane protein 115 | 0.043382452 |
| | | [Source: HGNC Symbol; Acc: HGNC: 30055] | |
| ENSG00000179632 | MAF1 | “MAF1 homolog, negative regulator of RNA | 0.043386409 |
| | | polymerase III | |
| | | [Source: HGNC Symbol; Acc: HGNC: 24966]” | |
| ENSG00000254910 | | | 0.043544967 |
| ENSG00000073111 | MCM2 | minichromosome maintenance complex component 2 | 0.043556269 |
| | | [Source: HGNC Symbol; Acc: HGNC: 6944] | |
| ENSG00000105698 | USF2 | “upstream transcription factor 2, c-fos interacting | 0.043737282 |
| | | [Source: HGNC Symbol; Acc: HGNC: 12594]” | |
| ENSG00000261043 | | | 0.043870446 |
| ENSG00000078808 | SDF4 | stromal cell derived factor 4 | 0.043914575 |
| | | [Source: HGNC Symbol; Acc: HGNC: 24188] | |
| ENSG00000182154 | MRPL41 | mitochondrial ribosomal protein L41 | 0.044022575 |
| | | [Source: HGNC Symbol; Acc: HGNC: 14492] | |
| ENSG00000237476 | LINC01637 | long intergenic non-protein coding RNA 1637 | 0.044022575 |
| | | [Source: HGNC Symbol; Acc: HGNC: 52424] | |
| ENSG00000264577 | | | 0.044022575 |
| ENSG00000160877 | NACC1 | nucleus accumbens associated 1 | 0.044046233 |
| | | [Source: HGNC Symbol; Acc: HGNC: 20967] | |
| ENSG00000162032 | SPSB3 | splA/ryanodine receptor domain and SOCS box | 0.044046233 |
| | | containing 3 | |
| | | [Source: HGNC Symbol; Acc: HGNC: 30629] | |
| ENSG00000123933 | MXD4 | MAX dimerization protein 4 | 0.044146857 |
| | | [Source: HGNC Symbol; Acc: HGNC: 13906] | |
| ENSG00000149260 | CAPN5 | calpain 5 | 0.044146857 |
| | | [Source: HGNC Symbol; Acc: HGNC: 1482] | |
| ENSG00000197324 | LRP10 | LDL receptor related protein 10 | 0.044146857 |
| | | [Source: HGNC Symbol; Acc: HGNC: 14553] | |
| ENSG00000128011 | LRFN1 | leucine rich repeat and fibronectin type III | 0.044424476 |
| | | domain containing 1 | |
| | | [Source: HGNC Symbol; Acc: HGNC: 29290] | |
| ENSG00000103126 | AXIN1 | axin 1 | 0.044490553 |
| | | [Source: HGNC Symbol; Acc: HGNC: 903] | |
| ENSG00000183134 | PTGDR2 | prostaglandin D2 receptor 2 | 0.044490553 |
| | | [Source: HGNC Symbol; Acc: HGNC: 4502] | |
| ENSG00000185905 | C16orf54 | chromosome 16 open reading frame 54 | 0.044574634 |
| | | [Source: HGNC Symbol; Acc: HGNC: 26649] | |
| ENSG00000185905 | C16orf54 | chromosome 16 open reading frame 54 | 0.044574634 |
| | | [Source: HGNC Symbol; Acc: HGNC: 26649] | |
| ENSG00000176974 | SHMT1 | serine hydroxymethyltransferase 1 | 0.044717969 |
| | | [Source: HGNC Symbol; Acc: HGNC: 10850] | |
| ENSG00000105327 | BBC3 | BCL2 binding component 3 | 0.044899327 |
| | | [Source: HGNC Symbol; Acc: HGNC: 17868] | |
| ENSG00000212123 | PRR22 | proline rich 22 | 0.044899327 |
| | | [Source: HGNC Symbol; Acc: HGNC: 28354] | |
| ENSG00000171798 | KNDC1 | kinase non-catalytic C-lobe domain containing 1 | 0.045139854 |
| | | [Source: HGNC Symbol; Acc: HGNC: 29374] | |
| ENSG00000073150 | PANX2 | pannexin 2 | 0.045237232 |
| | | [Source: HGNC Symbol; Acc: HGNC: 8600] | |
| ENSG00000126453 | BCL2L12 | BCL2 like 12 | 0.04527356 |
| | | [Source: HGNC Symbol; Acc: HGNC: 13787] | |
| ENSG00000159692 | CTBP1 | C-terminal binding protein 1 | 0.04527356 |
| | | [Source: HGNC Symbol; Acc: HGNC: 2494] | |
| ENSG00000105248 | CCDC94 | coiled-coil domain containing 94 | 0.045450088 |
| | | [Source: HGNC Symbol; Acc: HGNC: 25518] | |
| ENSG00000101220 | C20orf27 | chromosome 20 open reading frame 27 | 0.045474144 |
| | | [Source: HGNC Symbol; Acc: HGNC: 15873] | |
| ENSG00000188735 | TMEM120B | transmembrane protein 120B | 0.045474144 |
| | | [Source: HGNC Symbol; Acc: HGNC: 32008] | |
| ENSG00000149476 | TKFC | triokinase and FMN cyclase | 0.045497488 |
| | | [Source: HGNC Symbol; Acc: HGNC: 24552] | |
| ENSG00000100105 | PATZ1 | POZ/BTB and AT hook containing zinc finger 1 | 0.045500961 |
| | | [Source: HGNC Symbol; Acc: HGNC: 13071] | |
| ENSG00000136295 | TTYH3 | tweety family member 3 | 0.045528908 |
| | | [Source: HGNC Symbol; Acc: HGNC: 22222] | |
| ENSG00000104823 | ECH1 | enoyl-CoA hydratase 1 | 0.045555559 |
| | | [Source: HGNC Symbol; Acc: HGNC: 3149] | |
| ENSG00000167930 | FAM234A | family with sequence similarity 234 member A | 0.045584551 |
| | | [Source: HGNC Symbol; Acc: HGNC: 14163] | |
| ENSG00000125505 | MBOAT7 | membrane bound O-acyltransferase domain | 0.045601783 |
| | | containing 7 | |
| | | [Source: HGNC Symbol; Acc: HGNC: 15505] | |
| ENSG00000136720 | HS6ST1 | heparan sulfate 6-O-sulfotransferase 1 | 0.045601783 |
| | | [Source: HGNC Symbol; Acc: HGNC: 5201] | |
| ENSG00000156853 | ZNF689 | zinc finger protein 689 | 0.045601783 |
| | | [Source: HGNC Symbol; Acc: HGNC: 25173] | |
| ENSG00000176108 | CHMP6 | charged multivesicular body protein 6 | 0.045601783 |
| | | [Source: HGNC Symbol; Acc: HGNC: 25675] | |
| ENSG00000205336 | ADGRG1 | adhesion G protein-coupled receptor G1 | 0.045601783 |
| | | [Source: HGNC Symbol; Acc: HGNC: 4512] | |
| ENSG00000186350 | RXRA | retinoid X receptor alpha | 0.045691236 |
| | | [Source: HGNC Symbol; Acc: HGNC: 10477] | |
| ENSG00000164849 | GPR146 | G protein-coupled receptor 146 | 0.045781 |
| | | [Source: HGNC Symbol; Acc: HGNC: 21718] | |
| ENSG00000124313 | IQSEC2 | IQ motif and Sec7 domain 2 | 0.045921742 |
| | | [Source: HGNC Symbol; Acc: HGNC: 29059] | |
| ENSG00000148341 | SH3GLB2 | “SH3 domain containing GRB2 like, endophilin B2 | 0.045931004 |
| | | [Source: HGNC Symbol; Acc: HGNC: 10834]” | |
| ENSG00000213654 | GPSM3 | G protein signaling modulator 3 | 0.045963746 |
| | | [Source: HGNC Symbol; Acc: HGNC: 13945] | |
| ENSG00000171219 | CDC42BPG | CDC42 binding protein kinase gamma | 0.046017697 |
| | | [Source: HGNC Symbol; Acc: HGNC: 29829] | |
| ENSG00000125741 | OPA3 | “OPA3, outer mitochondrial membrane lipid | 0.046210538 |
| | | metabolism regulator | |
| | | [Source: HGNC Symbol; Acc: HGNC: 8142]” | |
| ENSG00000089693 | MLF2 | myeloid leukemia factor 2 | 0.046269835 |
| | | [Source: HGNC Symbol; Acc: HGNC: 7126] | |
| ENSG00000267283 | | | 0.046536749 |
| ENSG00000238164 | TNFRSF14-AS1 | TNFRSF14 antisense RNA 1 | 0.046584081 |
| | | [Source: HGNC Symbol; Acc: HGNC: 26966] | |
| ENSG00000167815 | PRDX2 | peroxiredoxin 2 | 0.046640005 |
| | | [Source: HGNC Symbol; Acc: HGNC: 9353] | |
| ENSG00000140564 | FURIN | “furin, paired basic amino acid cleaving enzyme | 0.046653045 |
| | | [Source: HGNC Symbol; Acc: HGNC: 8568]” | |
| ENSG00000059122 | FLYWCH1 | FLYWCH-type zinc finger 1 | 0.046934685 |
| | | [Source: HGNC SymbohAcc: HGNC: 25404] | |
| ENSG00000070423 | RNF126 | ring finger protein 126 | 0.047070015 |
| | | [Source: HGNC Symbol: Acc: HGNC: 21151] | |
| ENSG00000027847 | B4GALT7 | “beta-1,4-galactosyltransferase 7 | 0.047130774 |
| | | [Source: HGNC Symbol: Acc: HGNC: 930]” | |
| ENSG00000108518 | PFN1 | profilin 1 | 0.047157356 |
| | | [Source: HGNC Symbol: Acc: HGNC: 8881] | |
| ENSG00000110711 | AIP | aryl hydrocarbon receptor interacting protein | 0.04721490 |
| | | [Source: HGNC Symbol; Acc; HGNC: 358] | |
| ENSG00000164068 | RNF123 | ring finger protein 123 | 0.047214902 |
| | | [Source: HGNC Symbol: Acc: HGNC: 21148] | |
| ENSG00000137216 | TMEM63B | transmembrane protein 63 B | 0.047244731 |
| | | [Source: HGNC Symbol: Acc: HGNC: 17735] | |
| ENSG00000179588 | ZFPM1 | “zinc finger protein, FOG family member 1 | 0.047461433 |
| | | [Source: HGNC Symbol: Acc: HGNC: 19762]” | |
| ENSG00000188070 | C11orf95 | chromosome 11 open reading frame 95 | 0.047461433 |
| | | [Source: HGNC Symbol; Acc: HGNC: 28449] | |
| ENSG00000162722 | TRIM58 | tripartite motif containing 58 | 0.047475759 |
| | | [Source: HGNC Symbol; Acc: HGNC: 24150] | |
| ENSG00000068724 | TTC7A | tetratricopeptide repeat domain 7A | 0.0476766 |
| | | [Source: HGNC Symbol; Acc: HGNC: 19750] | |
| ENSG00000142227 | EMP3 | epithelial membrane protein 3 | 0.0476766 |
| | | [Source: HGNC Symbol; Acc: HGNC: 3335] | |
| ENSG00000159714 | ZDHHC1 | zinc finger DHHC-type containing 1 | 0.04772167 |
| | | [Source: HGNC Symbol; Acc: HGNC: 17916] | |
| ENSG00000196182 | STK40 | serine/threonine kinase 40 | 0.047779011 |
| | | [Source: HGNC Symbol; Acc: HGNC: 21373] | |
| ENSG00000099875 | MKNK2 | MAP kinase interacting serine/threonine kinase 2 | 0.047824795 |
| | | [Source: HGNC Symbol; Acc: HGNC: 7111] | |
| ENSG00000132514 | CLEC10A | C-type lectin domain containing 10A | 0.047824795 |
| | | [Source: HGNC Symbol; Acc: HGNC: 16916] | |
| ENSG00000197982 | C1orf122 | chromosome 1 open reading frame 122 | 0.047824795 |
| | | [Source: HGNC Symbol; Acc: HGNC: 24789] | |
| ENSG00000204348 | DXO | decapping exoribonuclease | 0.047913021 |
| | | [Source: HGNC Symbol; Acc: HGNC: 2992] | |
| ENSG00000259856 | RAB43P1 | RAB43 pseudogene 1 | 0.048194625 |
| | | [Source: HGNC Symbol; Acc: HGNC: 33153] | |
| ENSG00000103254 | FAM173A | family with sequence similarity 173 member A | 0.048206847 |
| | | [Source: HGNC Symbol; Acc: HGNC: 14152] | |
| ENSG00000196453 | ZNF777 | zinc finger protein 777 | 0.048246269 |
| | | [Source: HGNC Symbol; Acc: HGNC: 22213] | |
| ENSG00000167173 | C15orf39 | chromosome 15 open reading frame 39 | 0.048259142 |
| | | [Source: HGNC Symbol; Acc: HGNC: 24497] | |
| ENSG00000172534 | HCFC1 | host cell factor C1 | 0.04840783 |
| | | [Source: HGNC Symbol; Acc: HGNC: 4839] | |
| ENSG00000198804 | MT-CO1 | mitochondrially encoded cytochrome c oxidase I | 0.048409706 |
| | | [Source: HGNC Symbol; Acc: HGNC: 7419] | |
| ENSG00000244486 | SCARF2 | scavenger receptor class F member 2 | 0.048617065 |
| | | [Source: HGNC Symbol; Acc: HGNC: 19869] | |
| ENSG00000125652 | ALKBH7 | alkB homolog 7 | 0.048746819 |
| | | [Source: HGNC Symbol; Acc: HGNC: 21306] | |
| ENSG00000176101 | SSNA1 | SS nuclear autoantigen 1 | 0.048934402 |
| | | [Source: HGNC Symbol; Acc: HGNC: 11321] | |
| ENSG00000129103 | SUMF2 | sulfatase modifying factor 2 | 0.048950286 |
| | | [Source: HGNC Symbol; Acc: HGNC: 20415] | |
| ENSG00000115756 | HPCAL1 | hippocalcin like 1 | 0.049011448 |
| | | [Source: HGNC Symbol; Acc: HGNC: 5145] | |
| ENSG00000243566 | UPK3B | uroplakin 3B | 0.049012128 |
| | | [Source: HGNC Symbol; Acc: HGNC: 21444] | |
| ENSG00000149150 | SLC43A1 | solute carrier family 43 member 1 | 0.049128197 |
| | | [Source: HGNC Symbol; Acc: HGNC: 9225] | |
| ENSG00000103227 | LMF1 | lipase maturation factor 1 | 0.049193782 |
| | | [Source: HGNC Symbol; Acc: HGNC: 14154] | |
| ENSG00000271959 | | | 0.049311473 |
| ENSG00000261222 | | | 0.049471704 |
| ENSG00000126217 | MCF2L | MCF.2 cell line derived transforming sequence like | 0.049648177 |
| | | [Source: HGNC Symbol; Acc: HGNC: 14576] | |
| ENSG00000104964 | AES | amino-terminal enhancer of split | 0.049726662 |
| | | [Source: HGNC Symbol; Acc: HGNC: 307] | |
| ENSG00000127831 | VIL1 | villin 1 | 0.049726662 |
| | | [Source: HGNC Symbol; Acc: HGNC: 12690] | |
| ENSG00000169738 | DCXR | dicarbonyl and L-xylulose reductase | 0.049760492 |
| | | [Source: HGNC Symbol; Acc: HGNC: 18985] | |
| ENSG00000086015 | MAST2 | microtubule associated serine/threonine kinase 2 | 0.04998097 |
| | | [Source: HGNC Symbol; Acc: HGNC: 19035] | |

TABLE 3
AC3 GENES

| Ensembl | Symbol | Description | Score?/AUC? |
|---|---|---|---|
| ENSG00000251705 | RNA5-8SP6 | “RNA, 5.8S ribosomal pseudogene 6 | 5.29E−67 |
| | | [Source: HGNC Symbol; Acc: HGNC: 41960]” | |
| ENSG00000133110 | POSTN | periostin | 1.78E−06 |
| | | [Source: HGNC Symbol; Acc: HGNC: 16953] | |
| ENSG00000142449 | FBN3 | fibrillin 3 | 2.85E−05 |
| | | [Source: HGNC Symbol; Acc: HGNC: 18794] | |
| ENSG00000183668 | PSG9 | pregnancy specific beta-1-glycoprotein 9 | 9.26E−05 |
| | | [Source: HGNC Symbol; Acc: HGNC: 9526] | |
| ENSG00000258628 | | | 0.000107247 |
| ENSG00000260290 | | | 0.000163871 |
| ENSG00000224367 | OACYLP | “O-acyltransferase like, pseudogene | 0.000275943 |
| | | [Source: HGNC Symbol; Acc: HGNC: 44362]” | |
| ENSG00000108018 | SORCS1 | sortilin related VPS 10 domain containing receptor 1 | 0.000366056 |
| | | [Source: HGNC Symbol; Acc: HGNC: 16697] | |
| ENSG00000135454 | B4GALNT1 | “beta-1,4-N-acetyl-galactosaminyltransferase 1 | 0.000429017 |
| | | [Source: HGNC Symbol; Acc: HGNC: 4117]” | |
| ENSG00000266172 | NA | NA | 0.000574378 |
| ENSG00000173769 | TOPAZ1 | testis and ovary specific PAZ domain containing 1 | 0.00059023 |
| | | [Source: HGNC Symbol; Acc: HGNC: 24746] | |
| ENSG00000181378 | CFAP65 | cilia and flagella associated protein 65 | 0.000602774 |
| | | [Source: HGNC Symbol; Acc: HGNC: 25325] | |
| ENSG00000174498 | IGDCC3 | immunoglobulin superfamily DCC subclass member 3 | 0.00062895 |
| | | [Source: HGNC Symbol; Acc: HGNC: 9700] | |
| ENSG00000089116 | LHX5 | LIM homeobox 5 | 0.000639272 |
| | | [Source: HGNC Symbol; Acc: HGNC: 14216] | |
| ENSG00000161270 | NPHS1 | “NPHS1, nephrin | 0.000697583 |
| | | [Source: HGNC Symbol; Acc: HGNC: 7908]” | |
| ENSG00000006210 | CX3CL1 | C-X3-C motif chemokine ligand 1 | 0.000730947 |
| | | [Source: HGNC Symbol; Acc: HGNC: 10647] | |
| ENSG00000249618 | LINC02465 | long intergenic non-protein coding RNA 2465 | 0.000730947 |
| | | [Source: HGNC Symbol; Acc: HGNC: 53403] | |
| ENSG00000253871 | | | 0.000789684 |
| ENSG00000130540 | SULT4A1 | sulfotransferase family 4A member 1 | 0.000791019 |
| | | [Source: HGNC Symbol; Acc: HGNC: 14903] | |
| ENSG00000148942 | SLC5A12 | solute carrier family 5 member 12 | 0.000819378 |
| | | [Source: HGNC Symbol; Acc: HGNC: 28750] | |
| ENSG00000226790 | HNRNPA3P1 | heterogeneous nuclear ribonucleoprotein A3 | 0.000826721 |
| | | pseudogene 1 | |
| | | [Source: HGNC Symbol; Acc: HGNC: 13729] | |
| ENSG00000160460 | SPTBN4 | “spectrin beta, non-erythrocytic 4 | 0.000837162 |
| | | [Source: HGNC Symbol; Acc: HGNC: 14896]” | |
| ENSG00000243130 | PSG11 | pregnancy specific beta-1-glycoprotein 11 | 0.000837162 |
| | | [Source: HGNC Symbol; Acc: HGNC: 9516] | |
| ENSG00000244694 | PTCHD4 | patched domain containing 4 | 0.000837162 |
| | | [Source: HGNC Symbol; Acc: HGNC: 21345] | |
| ENSG00000249464 | LINC01091 | long intergenic non-protein coding RNA 1091 | 0.000837162 |
| | | [Source: HGNC Symbol; Acc: HGNC: 27721] | |
| ENSG00000011677 | GABRA3 | gamma-aminobutyric acid type A receptor | 0.000896018 |
| | | alpha3 subunit | |
| | | [Source: HGNC Symbol; Acc: HGNC: 4077] | |
| ENSG00000256343 | | | 0.00097441 |
| ENSG00000039139 | DNAH5 | dynein axonemal heavy chain 5 | 0.001236713 |
| | | [Source: HGNC Symbol; Acc: HGNC: 2950] | |
| ENSG00000130635 | COL5A1 | collagen type V alpha 1 chain | 0.001236713 |
| | | [Source: HGNC Symbol; Acc: HGNC: 2209] | |
| ENSG00000242512 | LINC01206 | long intergenic non-protein coding RNA 1206 | 0.001253551 |
| | | [Source: HGNC Symbol; Acc: HGNC: 49637] | |
| ENSG00000164692 | COL1A2 | collagen type I alpha 2 chain | 0.00126185 |
| | | [Source: HGNC Symbol; Acc: HGNC: 2198] | |
| ENSG00000259841 | LINC01566 | long intergenic non-protein coding RNA 1566 | 0.001274332 |
| | | [Source: HGNC Symbol; Acc: HGNC: 27555] | |
| ENSG00000170777 | TPD52L3 | tumor protein D52 like 3 | 0.001484087 |
| | | [Source: HGNC Symbol; Acc: HGNC: 23382] | |
| ENSG00000186487 | MYT1L | myelin transcription factor 1 like | 0.001774703 |
| | | [Source: HGNC Symbol; Acc: HGNC: 7623] | |
| ENSG00000082175 | PGR | progesterone receptor | 0.001879127 |
| | | [Source: HGNC Symbol; Acc: HGNC: 8910] | |
| ENSG00000205922 | ONECUT3 | one cut homeobox 3 | 0.001993667 |
| | | [Source: HGNC Symbol; Acc: HGNC: 13399] | |
| ENSG00000249341 | | | 0.002126073 |
| ENSG00000139865 | TTC6 | tetratricopeptide repeat domain 6 | 0.002268239 |
| | | [Source: HGNC Symbol; Acc: HGNC: 19739] | |
| ENSG00000154478 | GPR26 | G protein-coupled receptor 26 | 0.002268239 |
| | | [Source: HGNC Symbol; Acc: HGNC: 4481] | |
| ENSG00000174358 | SLC6A19 | solute carrier family 6 member 19 | 0.002268239 |
| | | [Source: HGNC Symbol; Acc: HGNC: 27960] | |
| ENSG00000235711 | ANKRD34C | ankyrin repeat domain 34C | 0.002268239 |
| | | [Source: HGNC Symbol; Acc: HGNC: 33888] | |
| ENSG00000170381 | SEMA3E | semaphorin 3E | 0.002327494 |
| | | [Source: HGNC Symbol; Acc: HGNC: 10727] | |
| ENSG00000142611 | PRDM16 | PR/SET domain 16 | 0.002350766 |
| | | [Source: HGNC Symbol; Acc: HGNC: 14000] | |
| ENSG00000205396 | LINC00661 | long intergenic non-protein coding RNA 661 | 0.002366966 |
| | | [Source: HGNC Symbol: Acc: HGNC: 27002] | |
| ENSG00000253288 | | | 0.00239082 |
| ENSG00000171435 | KSR2 | kinase suppressor of ras 2 | 0.002607962 |
| | | [Source: HGNC Symbol: Acc: HGNC: 18610] | |
| ENSG00000256616 | | | 0.002639596 |
| ENSG00000171804 | WDR87 | WD repeat domain 87 | 0.002806683 |
| | | [Source: HGNC Symbol: Acc: HGNC: 29934] | |
| ENSG00000237125 | HAND2-AS1 | HAND2 antisense RNA 1 (head to head) | 0.00289999 |
| | | [Source: HGNC Symbol: Acc: HGNC: 48872] | |
| ENSG00000240694 | PNMA2 | PNMA family member 2 | 0.002940811 |
| | | [Source: HGNC Symbol: Acc: HGNC: 9159] | |
| ENSG00000102452 | NALCN | sodium leak channel, non-selective | 0.003094645 |
| | | [Source: HGNC Symbol; Acc: HGNC: 19082]” | |
| ENSG00000214929 | SPATA31D1 | SPATA31 subfamily D member 1 | 0.003264148 |
| | | [Source: HGNC Symbol; Acc: HGNC: 37283] | |
| ENSG00000115041 | KCNIP3 | potassium voltage-gated channel interacting protein 3 | 0.00328034 |
| | | [Source: HGNC Symbol; Acc: HGNC: 15523] | |
| ENSG00000185038 | MROH2A | maestro heat like repeat family member 2A | 0.003313878 |
| | | [Source: HGNC Symbol; Acc: HGNC: 27936] | |
| ENSG00000138892 | TTLL8 | tubulin tyrosine ligase like 8 | 0.003497603 |
| | | [Source: HGNC Symbol; Acc: HGNC: 34000] | |
| ENSG00000147573 | TRIM55 | tripartite motif containing 55 | 0.003573936 |
| | | [Source: HGNC Symbol; Acc: HGNC: 14215] | |
| ENSG00000165323 | FAT3 | FAT atypical cadherin 3 | 0.003629544 |
| | | [Source: HGNC Symbol; Acc: HGNC: 23112] | |
| ENSG00000142623 | PADI1 | peptidyl arginine deiminase 1 | 0.003697796 |
| | | [Source: HGNC Symbol; Acc: HGNC: 18367] | |
| ENSG00000146521 | LINC01558 | long intergenic non-protein coding RNA 1558 | 0.003779666 |
| | | [Source: HGNC Symbol; Acc: HGNC: 21235] | |
| ENSG00000125255 | SLC10A2 | solute carrier family 10 member 2 | 0.003857127 |
| | | [Source: HGNC Symbol; Acc: HGNC: 10906] | |
| ENSG00000103855 | CD276 | CD276 molecule | 0.004037668 |
| | | [Source: HGNC Symbol; Acc: HGNC: 19137] | |
| ENSG00000168907 | PLA2G4F | phospholipase A2 group IVF | 0.00413675 |
| | | [Source: HGNC Symbol; Acc: HGNC: 27396] | |
| ENSG00000141668 | CBLN2 | cerebellin 2 precursor | 0.004198501 |
| | | [Source: HGNC Symbol; Acc: HGNC: 1544] | |
| ENSG00000197991 | | | 0.004252179 |
| ENSG00000149633 | KIAA1755 | KIAA1755 | 0.004331797 |
| | | [Source: HGNC Symbol; Acc: HGNC: 29372] | |
| ENSG00000157927 | RADIL | Rap associating with DEL domain | 0.004332718 |
| | | [Source: HGNC Symbol; Acc: HGNC: 22226] | |
| ENSG00000138759 | FRAS1 | Fraser extracellular matrix complex subunit 1 | 0.004539725 |
| | | [Source: HGNC Symbol; Acc: HGNC: 19185] | |
| ENSG00000174963 | ZIC4 | Zic family member 4 | 0.004539725 |
| | | [Source: HGNC Symbol; Acc: HGNC: 20393] | |
| ENSG00000177551 | NHLH2 | nescient helix-loop-helix 2 | 0.004560802 |
| | | [Source: HGNC Symbol; Acc: HGNC: 7818] | |
| ENSG00000250230 | | | 0.004576049 |
| ENSG00000204929 | | | 0.00461274 |
| ENSG00000163975 | MELTF | melanotransferrin | 0.004655716 |
| | | [Source: HGNC Symbol; Acc: HGNC: 7037] | |
| ENSG00000095587 | TLL2 | tolloid like 2 | 0.004686524 |
| | | [Source: HGNC Symbol: Acc: HGNC: 11844] | |
| ENSG00000221826 | PSG3 | pregnancy specific beta-1-glycoprotein 3 | 0.004686524 |
| | | [Source: HGNC Symbol; Acc: HGNC: 9520] | |
| ENSG00000105392 | CRX | cone-rod homeobox | 0.004730282 |
| | | [Source: HGNC Symbol; Acc: HGNC: 2383] | |
| ENSG00000188338 | SLC38A3 | solute carrier family 38 member 3 | 0.004737254 |
| | | [Source: HGNC Symbol; Acc: HGNC: 18044] | |
| ENSG00000167654 | ATCAY | “ATCAY, caytaxin | 0.004891089 |
| | | [Source: HGNC Symbol; Acc: HGNC: 779]” | |
| ENSG00000177511 | ST8SIA3 | “ST8 alpha-N-acetyl-neuraminide | 0.00498476 |
| | | alpha-2,8-sialyltransferase 3 | |
| | | [Source: HGNC Symbol; Acc: HGNC: 14269]” | |
| ENSG00000215895 | | | 0.005091461 |
| ENSG00000124466 | LYPD3 | LY6/PLAUR domain containing 3 | 0.005118791 |
| | | [Source: HGNC Symbol; Acc: HGNC: 24880] | |
| ENSG00000084636 | COL16A1 | collagen type XVI alpha 1 chain | 0.00516436 |
| | | [Source: HGNC Symbol; Acc: HGNC: 2193] | |
| ENSG00000104537 | ANXA13 | annexin A13 | 0.005166641 |
| | | [Source: HGNC Symbol; Acc: HGNC: 536] | |
| ENSG00000145526 | CDH18 | cadherin 18 | 0.005245896 |
| | | [Source: HGNC Symbol; Acc: HGNC: 1757] | |
| ENSG00000161103 | | | 0.005294938 |
| ENSG00000168484 | SFTPC | surfactant protein C | 0.005473496 |
| | | [Source: HGNC Symbol; Acc: HGNC: 10802] | |
| ENSG00000188886 | ASTL | astacin like metalloendopeptidase | 0.005530506 |
| | | [Source: HGNC Symbol; Acc: HGNC: 31704] | |
| ENSG00000198765 | SYCP1 | synaptonemal complex protein 1 | 0.005554714 |
| | | [Source: HGNC Symbol; Acc: HGNC: 11487] | |
| ENSG00000234177 | LINC01114 | long intergenic non-protein coding RNA 1114 | 0.005808942 |
| | | [Source: HGNC Symbol; Acc: HGNC: 49245] | |
| ENSG00000091656 | ZFHX4 | zinc finger homeobox 4 | 0.005809417 |
| | | [Source: HGNC Symbol; Acc: HGNC: 30939] | |
| ENSG00000151572 | ANO4 | anoctamin 4 | 0.005857886 |
| | | [Source: HGNC Symbol; Acc: HGNC: 23837] | |
| ENSG00000178965 | ERICH3 | glutamate rich 3 | 0.005890875 |
| | | [Source: HGNC Symbol; Acc: HGNC: 25346] | |
| ENSG00000248587 | GDNF-AS1 | GDNF antisense RNA 1 (head to head) | 0.005890875 |
| | | [Source: HGNC Symbol; Acc: HGNC: 43592] | |
| ENSG00000144908 | ALDH1L1 | aldehyde dehydrogenase 1 family member L1 | 0.006015103 |
| | | [Source: HGNC Symbol; Acc: HGNC: 3978] | |
| ENSG00000152822 | GRM1 | glutamate metabotropic receptor 1 | 0.006033529 |
| | | [Source: HGNC Symbol; Acc: HGNC: 4593] | |
| ENSG00000138675 | FGF5 | fibroblast growth factor 5 | 0.006101348 |
| | | [Source: HGNC Symbol; Acc: HGNC: 3683] | |
| ENSG00000187772 | LIN28B | lin-28 homolog B | 0.006111478 |
| | | [Source: HGNC Symbol; Acc: HGNC: 32207] | |
| ENSG00000227471 | AKR1B15 | aldo-keto reductase family 1 member B15 | 0.006366933 |
| | | [Source: HGNC Symbol; Acc: HGNC: 37281] | |
| ENSG00000174502 | SLC26A9 | solute carrier family 26 member 9 | 0.006716425 |
| | | [Source: HGNC Symbol; Acc: HGNC: 14469] | |
| ENSG00000078549 | ADCYAP1R1 | ADCYAP receptor type I | 0.006848491 |
| | | [Source: HGNC Symbol; Acc: HGNC: 242] | |
| ENSG00000159650 | UROC1 | urocanate hydratase 1 | 0.006848491 |
| | | [Source: HGNC Symbol; Acc: HGNC: 26444] | |
| ENSG00000217094 | PPIAP31 | peptidylprolyl isomerase A pseudogene 31 | 0.006866623 |
| | | [Source: HGNC Symbol; Acc: HGNC: 44962] | |
| ENSG00000006128 | TAC1 | tachykinin precursor 1 | 0.006929673 |
| | | [Source: HGNC Symbol; Acc: HGNC: 11517] | |
| ENSG00000158077 | NLRP14 | NLR family pyrin domain containing 14 | 0.006952696 |
| | | [Source: HGNC Symbol; Acc: HGNC: 22939] | |
| ENSG00000223414 | LINC00473 | long intergenic non-protein coding RNA 473 | 0.006952696 |
| | | [Source: HGNC Symbol; Acc: HGNC: 21160] | |
| ENSG00000144488 | ESPNL | espin like | 0.007039881 |
| | | [Source: HGNC Symbol; Acc: HGNC: 27937] | |
| ENSG00000144730 | IL17RD | interleukin 17 receptor D | 0.007141116 |
| | | [Source: HGNC Symbol; Acc: HGNC: 17616] | |
| ENSG00000137819 | PAQR5 | progestin and adipoQ receptor family member 5 | 0.007179081 |
| | | [Source: HGNC Symbol; Acc: HGNC: 29645] | |
| ENSG00000162631 | NTNG1 | netrin G1 | 0.007255358 |
| | | [Source: HGNC Symbol: Acc: HGNC: 23319] | |
| ENSG00000185974 | GRK1 | G protein-coupled receptor kinase 1 | 0.007327997 |
| | | [Source: HGNC Symbol; Acc: HGNC: 10013] | |
| ENSG00000261275 | | | 0.007327997 |
| ENSG00000249267 | LINC00939 | long intergenic non-protein coding RNA 939 | 0.007349367 |
| | | [Source: HGNC Symbol; Acc: HGNC: 48631] | |
| ENSG00000227827 | | | 0.007403828 |
| ENSG00000100065 | CARD10 | caspase recruitment domain family member 10 | 0.007527421 |
| | | [Source: HGNC Symbol: Acc: HGNC: 16422] | |
| ENSG00000119125 | GDA | guanine deaminase | 0.007619923 |
| | | [Source: HGNC Symbol: Acc: HGNC: 4212] | |
| ENSG00000106304 | SPAM1 | sperm adhesion molecule 1 | 0.00781679 |
| | | [Source: HGNC Symbol: Acc: HGNC: 11217] | |
| ENSG00000250493 | | | 0.007835589 |
| ENSG00000158258 | CLSTN2 | calsyntenin 2 | 0.008149414 |
| | | [Source: HGNC Symbol; Acc: HGNC: 17448] | |
| ENSG00000175329 | ISX | intestine specific homeobox | 0.008233566 |
| | | [Source: HGNC Symbol; Acc: HGNC: 28084] | |
| ENSG00000188488 | SERPINA5 | serpin family A member 5 | 0.008534971 |
| | | [Source: HGNC Symbol; Acc: HGNC: 8723] | |
| ENSG00000249584 | LINC02225 | long intergenic non-protein coding RNA 2225 | 0.00866593 |
| | | Source: HGNC Symbol; Acc: HGNC: 53094] | |
| ENSG00000147655 | RSPO2 | R-spondin 2 | 0.008823914 |
| | | [Source: HGNC Symbol; Acc: HGNC: 28583] | |
| ENSG00000171587 | DSCAM | DS cell adhesion molecule | 0.008841283 |
| | | [Source: HGNC Symbol; Acc: HGNC: 3039] | |
| ENSG00000120738 | EGR1 | early growth response 1 | 0.008960131 |
| | | [Source: HGNC Symbol; Acc: HGNC: 3238] | |
| ENSG00000127129 | EDN2 | endothelin 2 | 0.009244272 |
| | | [Source: HGNC Symbol; Acc: HGNC: 3177] | |
| ENSG00000157423 | HYDIN | “HYDIN, axonemal central pair apparatus protein | 0.009244272 |
| | | [Source: HGNC Symbol; Acc: HGNC: 19368]” | |
| ENSG00000196565 | HBG2 | hemoglobin subunit gamma 2 | 0.009327518 |
| | | [Source: HGNC Symbol; Acc: HGNC: 4832] | |
| ENSG00000235881 | | | 0.009327518 |
| ENSG00000111262 | KCNA1 | potassium voltage-gated channel subfamily A member 1 | 0.009418575 |
| | | [Source: HGNC Symbol; Acc: HGNC: 6218] | |
| ENSG00000187527 | ATP13A5 | ATPase 13A5 | 0.009514824 |
| | | [Source: HGNC Symbol; Acc: HGNC: 31789] | |
| ENSG00000188803 | SHISA6 | shisa family member 6 | 0.009514824 |
| | | [Source: HGNC Symbol; Acc: HGNC: 34491] | |
| ENSG00000175535 | PNLIP | pancreatic lipase | 0.009619326 |
| | | [Source: HGNC Symbol; Acc: HGNC: 9155] | |
| ENSG00000225953 | SATB2-AS1 | SATB2 antisense RNA 1 | 0.009647997 |
| | | [Source: HGNC Symbol; Acc: HGNC: 26490] | |
| ENSG00000136695 | IL36RN | interleukin 36 receptor antagonist | 0.009810065 |
| | | [Source: HGNC Symbol; Acc: HGNC: 15561] | |
| ENSG00000259790 | ANP32BP1 | acidic nuclear phosphoprotein 32 family member | 0.009820284 |
| | | B pseudogene 1 | |
| | | [Source: HGNC Symbol; Acc: HGNC: 24267] | |
| ENSG00000225813 | | | 0.009894409 |
| ENSG00000179008 | C14orf39 | chromosome 14 open reading frame 39 | 0.009896903 |
| | | [Source: HGNC Symbol; Acc: HGNC: 19849] | |
| ENSG00000150893 | FREM2 | FRAS1 related extracellular matrix protein 2 | 0.009945757 |
| | | [Source: HGNC Symbol; Acc: HGNC: 25396] | |
| ENSG00000197079 | KRT35 | keratin 35 | 0.009945757 |
| | | [Source: HGNC Symbol; Acc: HGNC: 6453] | |
| ENSG00000231131 | LINC01468 | long intergenic non-protein coding RNA 1468 | 0.010123625 |
| | | [Source: HGNC Symbol; Acc: HGNC: 50913] | |
| ENSG00000268388 | FENDRR | FOXF1 adjacent non-coding developmental | 0.010151023 |
| | | regulatory RNA | |
| | | [Source: HGNC Symbol; Acc: HGNC: 43894] | |
| ENSG00000159251 | ACTC1 | “actin, alpha, cardiac muscle 1 | 0.010212535 |
| | | [Source: HGNC Symbol; Acc: HGNC: 143]” | |
| ENSG00000158125 | XDH | xanthine dehydrogenase | 0.010258334 |
| | | [Source: HGNC Symbol; Acc: HGNC: 12805] | |
| ENSG00000156222 | SLC28A1 | solute carrier family 28 member 1 | 0.010392835 |
| | | [Source: HGNC Symbol; Acc: HGNC: 11001] | |
| ENSG00000260759 | | | 0.010741484 |
| ENSG00000110975 | SYT10 | synaptotagmin 10 | 0.010787993 |
| | | [Source: HGNC SymboliAcc: HGNC: 19266] | |
| ENSG00000186185 | KIF18B | kinesin family member 18B | 0.010844893 |
| | | [Source: HGNC Symbol; Acc: HGNC: 27102] | |
| ENSG00000110887 | DAO | D-amino acid oxidase | 0.011064297 |
| | | [Source: HGNC Symbol; Acc: HGNC: 2671] | |
| ENSG00000132297 | HHLA1 | HERV-H LTR-associating 1 | 0.011064297 |
| | | [Source: HGNC Symbol; Acc: HGNC: 4904] | |
| ENSG00000146839 | ZAN | zonadhesin (gene/pseudogene) | 0.011064297 |
| | | [Source: HGNC Symbol; Acc: HGNC: 12857] | |
| ENSG00000215864 | NBPF7 | NBPF member 7 | 0.01113278 |
| | | [Source: HGNC Symbol; Acc: HGNC: 31989] | |
| ENSG00000233395 | LINC00841 | long intergenic non-protein coding RNA 841 | 0.011213055 |
| | | [Source: HGNC Symbol; Acc: HGNC: 27430] | |
| ENSG00000177354 | C10orf71 | chromosome 10 open reading frame 71 | 0.011268372 |
| | | [Source: HGNC Symbol; Acc: HGNC: 26973] | |
| ENSG00000148357 | HMCN2 | hemicentin 2 | 0.01150342 |
| | | [Source: HGNC Symbol; Acc: HGNC: 21293] | |
| ENSG00000215405 | NA | NA | 0.011599767 |
| ENSG00000203900 | | | 0.011732314 |
| ENSG00000218672 | | | 0.011732314 |
| ENSG00000261104 | | | 0.011732314 |
| ENSG00000123243 | ITIH5 | inter-alpha-trypsin inhibitor heavy chain family | 0.011851589 |
| | | member 5 | |
| | | [Source: HGNC Symbol; Acc: HGNC: 21449] | |
| ENSG00000213467 | HMGB1P37 | high mobility group box 1 pseudogene 37 | 0.011876979 |
| | | [Source: HGNC Symbol; Acc: HGNC: 39184] | |
| ENSG00000119283 | TRIM67 | tripartite motif containing 67 | 0.011900585 |
| | | [Source: HGNC Symbol; Acc: HGNC: 31859] | |
| ENSG00000166984 | TCP10L2 | t-complex 10 like 2 | 0.012009181 |
| | | [Source: HGNC Symbol; Acc: HGNC: 21254] | |
| ENSG00000204941 | PSG5 | pregnancy specific beta-1-glycoprotein 5 | 0.012022323 |
| | | [Source: HGNC Symbol; Acc: HGNC: 9522] | |
| ENSG00000230552 | | | 0.012137266 |
| ENSG00000115155 | OTOF | otoferlin | 0.012228668 |
| | | [Source: HGNC Symbol; Acc: HGNC: 8515] | |
| ENSG00000163395 | IGFN1 | immunoglobulin-like and fibronectin type III | 0.012230791 |
| | | domain containing 1 | |
| | | [Source: HGNC Symbol; Acc: HGNC: 24607] | |
| ENSG00000122778 | KIAA1549 | KIAA1549 | 0.012393242 |
| | | [Source: HGNC Symbol; Acc: HGNC: 22219] | |
| ENSG00000169169 | CPT1C | carnitine palmitoyltransferase 1C | 0.012437719 |
| | | [Source: HGNC Symbol; Acc: HGNC: 18540] | |
| ENSG00000160994 | CCDC105 | coiled-coil domain containing 105 | 0.012486932 |
| | | [Source: HGNC Symbol; Acc: HGNC: 26866] | |
| ENSG00000237515 | SHISA9 | shisa family member 9 | 0.012486932 |
| | | [Source: HGNC Symbol; Acc: HGNC: 37231] | |
| ENSG00000105605 | CACNG7 | calcium voltage-gated channel auxiliary subunit | 0.012676606 |
| | | gamma 7 | |
| | | [Source: HGNC Symbol; Acc: HGNC: 13626] | |
| ENSG00000185739 | SRL | sarcalumenin | 0.012676606 |
| | | [Source: HGNC Symbol; Acc: HGNC: 11295] | |
| ENSG00000101680 | LAMA1 | laminin subunit alpha 1 | 0.012767139 |
| | | [Source: HGNC Symbol: Acc: HGNC: 6481] | |
| ENSG00000240021 | TEX35 | testis expressed 35 | 0.012795538 |
| | | [Source: HGNC Symbol: Acc: HGNC: 25366] | |
| ENSG00000250423 | KIAA1210 | KIAA1210 | 0.012935817 |
| | | [Source: HGNC Symbol: Acc: HGNC: 29218] | |
| ENSG00000198788 | MUC2 | “mucin 2, oligomeric mucus/gel-forming | 0.012968601 |
| | | [Source: HGNC Symbol; Acc: HGNC: 7512]” | |
| ENSG00000205312 | KRT17P4 | keratin 17 pseudogene 4 | 0.013016002 |
| | | [Source: HGNC Symbol; Acc: HGNC: 50722] | |
| ENSG00000214128 | TMEM213 | transmembrane protein 213 | 0.013016002 |
| | | [Source: HGNC Symbol; Acc: HGNC: 27220] | |
| ENSG00000178568 | ERBB4 | erb-b2 receptor tyrosine kinase 4 | 0.013045986 |
| | | [Source: HGNC Symbol; Acc: HGNC: 3432] | |
| ENSG00000175084 | DES | desmin | 0.013296119 |
| | | [Source: HGNC Symbol; Acc: HGNC: 2770] | |
| ENSG00000078295 | ADCY2 | adenylate cyclase 2 | 0.013400191 |
| | | [Source: HGNC Symbol; Acc: HGNC: 233] | |
| ENSG00000132639 | SNAP25 | synaptosome associated protein 25 | 0.013440348 |
| | | [Source: HGNC Symbol; Acc: HGNC: 11132] | |
| ENSG00000187094 | CCK | cholecystokinin | 0.013447765 |
| | | [Source: HGNC Symbol; Acc: HGNC: 1569] | |
| ENSG00000018625 | ATP1A2 | ATPase Na+/K+ transporting subunit alpha 2 | 0.013509365 |
| | | [Source: HGNC Symbol; Acc: HGNC: 800] | |
| ENSG00000168542 | COL3A1 | collagen type III alpha 1 chain | 0.013843652 |
| | | [Source: HGNC Symbol; Acc: HGNC: 2201] | |
| ENSG00000239921 | LINC01471 | long intergenic non-protein coding RNA 1471 | 0.013848913 |
| | | [Source: HGNC Symbol; Acc: HGNC: 51106] | |
| ENSG00000233183 | | | 0.013964273 |
| ENSG00000167798 | C3P1 | complement component 3 precursor pseudogene | 0.013987555 |
| | | [Source: HGNC Symbol; Acc: HGNC: 34414] | |
| ENSG00000183778 | B3GALT5 | “beta-1,3-galactosyltransferase 5 | 0.01405326 |
| | | [Source: HGNC Symbol; Acc: HGNC: 920]” | |
| ENSG00000168481 | LGI3 | leucine rich repeat LGI family member 3 | 0.014132769 |
| | | [Source: HGNC Symbol; Acc: HGNC: 18711] | |
| ENSG00000227744 | LINC01940 | long intergenic non-protein coding RNA 1940 | 0.014149077 |
| | | [Source: HGNC Symbol; Acc: HGNC: 52763] | |
| ENSG00000138162 | TACC2 | transforming acidic coiled-coil containing protein 2 | 0.014184675 |
| | | [Source: HGNC Symbol; Acc: HGNC: 11523] | |
| ENSG00000250049 | | | 0.014522615 |
| ENSG00000236445 | LINC00608 | long intergenic non-protein coding RNA 608 | 0.014606813 |
| | | Source: HGNC Symbol; Acc: HGNC: 27179] | |
| ENSG00000165966 | PDZRN4 | PDZ domain containing ring finger 4 | 0.014718872 |
| | | [Source: HGNC Symbol; Acc: HGNC: 30552] | |
| ENSG00000169876 | MUC17 | “mucin 17, cell surface associated | 0.014749092 |
| | | [Source: HGNC Symbol; Acc: HGNC: 16800]” | |
| ENSG00000078898 | BPIFB2 | BPI fold containing family B member 2 | 0.014831419 |
| | | [Source: HGNC Symbol; Acc: HGNC: 16177] | |
| ENSG00000130528 | HRC | histidine rich calcium binding protein | 0.014902982 |
| | | [Source: HGNC Symbol; Acc: HGNC: 5178] | |
| ENSG00000111799 | COL12A1 | collagen type XII alpha 1 chain | 0.015069239 |
| | | [Source: HGNC Symbol; Acc: HGNC: 2188] | |
| ENSG00000185303 | SFTPA2 | surfactant protein A2 | 0.015347263 |
| | | [Source: HGNC Symbol; Acc: HGNC: 10799] | |
| ENSG00000146648 | EGFR | epidermal growth factor receptor | 0.015466899 |
| | | [Source: HGNC Symbol; Acc: HGNC: 3236] | |
| ENSG00000205592 | MUC19 | “mucin 19, oligomeric | 0.015539471 |
| | | [Source: HGNC Symbol; Acc: HGNC: 14362]” | |
| ENSG00000198597 | ZNF536 | zinc finger protein 536 | 0.015552452 |
| | | [Source: HGNC Symbol; Acc: HGNC: 29025] | |
| ENSG00000120332 | TNN | tenascin N | 0.015559411 |
| | | [Source: HGNC Symbol; Acc: HGNC: 22942] | |
| ENSG00000197406 | DIO3 | iodothyronine deiodinase 3 | 0.015755832 |
| | | [Source: HGNC Symbol; Acc: HGNC: 2885] | |
| ENSG00000204283 | LINC01973 | long intergenic non-protein coding RNA 1973 | 0.015755832 |
| | | [Source: HGNC Symbol; Acc: HGNC: 52800] | |
| ENSG00000151224 | MAT1A | methionine adenosyltransferase 1A | 0.015829354 |
| | | [Source: HGNC Symbol; Acc: HGNC: 6903] | |
| ENSG00000257008 | GPR142 | G protein-coupled receptor 142 | 0.015942034 |
| | | [Source: HGNC Symbol; Acc: HGNC: 20088] | |
| ENSG00000139220 | PPFIA2 | PTPRF interacting protein alpha 2 | 0.015986308 |
| | | [Source: HGNC Symbol; Acc: HGNC: 9246] | |
| ENSG00000141946 | ZIM3 | zinc finger imprinted 3 | 0.015986308 |
| | | [Source: HGNC Symbol; Acc: HGNC: 16366] | |
| ENSG00000178171 | AMER3 | APC membrane recruitment protein 3 | 0.015986308 |
| | | [Source: HGNC Symbol; Acc: HGNC: 26771] | |
| ENSG00000232756 | | | 0.015986308 |
| ENSG00000130477 | UNC13A | unc-13 homolog A | 0.016007101 |
| | | [Source: HGNC Symbol; Acc: HGNC: 23150] | |
| ENSG00000070886 | EPHA8 | EPH receptor A8 | 0.016008143 |
| | | [Source: HGNC Symbol; Acc: HGNC: 3391] | |
| ENSG00000253301 | LINC01606 | long intergenic non-protein coding RNA 1606 | 0.016008143 |
| | | [Source: HGNC Symbol; Acc: HGNC: 51656] | |
| ENSG00000006788 | MYH13 | myosin heavy chain 13 | 0.01606756 |
| | | [Source: HGNC Symbol; Acc: HGNC: 7571] | |
| ENSG00000183287 | CCBE1 | collagen and calcium binding EGF domains 1 | 0.016243979 |
| | | [Source: HGNC Symbol; Acc: HGNC: 29426] | |
| ENSG00000262691 | | | 0.016243979 |
| ENSG00000125740 | FOSB | “FosB proto-oncogene, AP-1 transcription | 0.016263717 |
| | | factor subunit | |
| | | [Source: HGNC Symbol; Acc: HGNC: 3797]” | |
| ENSG00000133083 | DCLK1 | doublecortin like kinase 1 | 0.01630694 |
| | | [Source: HGNC Symbol; Acc: HGNC: 2700] | |
| ENSG00000144820 | ADGRG7 | adhesion G protein-coupled receptor G7 | 0.01630694 |
| | | [Source: HGNC Symbol; Acc: HGNC: 19241] | |
| ENSG00000178031 | ADAMTSL1 | ADAMTS like 1 | 0.016324743 |
| | | [Source: HGNC Symbol; Acc: HGNC: 14632] | |
| ENSG00000187905 | LRRC74B | leucine rich repeat containing 74B | 0.016416472 |
| | | [Source: HGNC Symbol; Acc: HGNC: 34301] | |
| ENSG00000221878 | PSG7 | pregnancy specific beta-1-glycoprotein 7 | 0.016416472 |
| | | (gene/pseudogene) | |
| | | [Source: HGNC Symbol; Acc: HGNC: 9524] | |
| ENSG00000254101 | LINC02055 | long intergenic non-protein coding RNA 2055 | 0.016416472 |
| | | [Source: HGNC Symbol; Acc: HGNC: 52895] | |
| ENSG00000120251 | GRIA2 | glutamate ionotropic receptor AMPA type subunit 2 | 0.016488676 |
| | | [Source: HGNC Symbol; Acc: HGNC: 4572] | |
| ENSG00000233991 | NA | NA | 0.016488676 |
| ENSG00000214402 | LCNL1 | lipocalin like 1 | 0.016554945 |
| | | [Source: HGNC Symbol; Acc: HGNC: 34436] | |
| ENSG00000224271 | | | 0.016611527 |
| ENSG00000257576 | HSPD1P4 | heat shock protein family D (Hsp60) member | 0.016611527 |
| | | 1 pseudogene 4 | |
| | | [Source: HGNC Symbol; Acc: HGNC: 35146] | |
| ENSG00000228549 | | | 0.016653065 |
| ENSG00000178645 | C10orf53 | chromosome 10 open reading frame 53 | 0.016654478 |
| | | [Source: HGNC Symbol; Acc: HGNC: 27421] | |
| ENSG00000100078 | PLA2G3 | phospholipase A2 group III | 0.016825197 |
| | | [Source: HGNC Symbol; Acc: HGNC: 17934] | |
| ENSG00000154099 | DNAAF1 | dynein axonemal assembly factor 1 | 0.016918546 |
| | | [Source: HGNC Symbol; Acc: HGNC: 30539] | |
| ENSG00000183242 | WT1-AS | WT1 antisense RNA | 0.016918546 |
| | | [Source: HGNC Symbol; Acc: HGNC: 18135] | |
| ENSG00000124253 | PCK1 | phosphoenolpyruvate carboxykinase 1 | 0.016968016 |
| | | [Source: HGNC Symbol; Acc: HGNC: 8724] | |
| ENSG00000183304 | FAM9A | family with sequence similarity 9 member A | 0.016968016 |
| | | [Source: HGNC SymbokAcc: HGNC: 18403] | |
| ENSG00000210127 | MT-TA | mitochondrially encoded tRNA alanine | 0.016968016 |
| | | [Source: HGNC Symbol; Acc: HGNC: 7475] | |
| ENSG00000258679 | | | 0.016968016 |
| ENSG00000130287 | NCAN | neurocan | 0.016985672 |
| | | [Source: HGNC Symbol; Acc: HGNC: 2465] | |
| ENSG00000088340 | FER1L4 | “fer-1 like family member 4, pseudogene | 0.017112355 |
| | | [Source: HGNC Symbol; Acc: HGNC: 15801]” | |
| ENSG00000196415 | PRTN3 | proteinase 3 | 0.017180036 |
| | | [Source: HGNC Symbol; Acc: HGNC: 9495] | |
| ENSG00000135917 | SLC19A3 | solute carrier family 19 member 3 | 0.017339051 |
| | | [Source: HGNC Symbol; Acc: HGNC: 16266] | |
| ENSG00000233539 | | | 0.017342649 |
| ENSG00000176584 | DMBT1P1 | deleted in malignant brain tumors 1 pseudogene 1 | 0.017392388 |
| | | [Source: HGNC Symbol; Acc: HGNC: 49497] | |
| ENSG00000135097 | MSI1 | musashi RNA binding protein 1 | 0.017394805 |
| | | [Source: HGNC Symbol; Acc: HGNC: 7330] | |
| ENSG00000091128 | LAMB4 | laminin subunit beta 4 | 0.017415673 |
| | | [Source: HGNC Symbol; Acc: HGNC: 6491] | |
| ENSG00000168367 | LINC00917 | long intergenic non-protein coding RNA 917 | 0.017415673 |
| | | [Source: HGNC Symbol; Acc: HGNC: 48607] | |
| ENSG00000224668 | IPO8P1 | importin 8 pseudogene 1 | 0.017704945 |
| | | [Source: HGNC Symbol; Acc: HGNC: 41955] | |
| ENSG00000165757 | JCAD | junctional cadherin 5 associated | 0.017712329 |
| | | [Source: HGNC Symbol; Acc: HGNC: 29283] | |
| ENSG00000166558 | SLC38A8 | solute carrier family 38 member 8 | 0.017722732 |
| | | [Source: HGNC Symbol; Acc: HGNC: 32434] | |
| ENSG00000185467 | KPNA7 | karyopherin subunit alpha 7 | 0.017767827 |
| | | [Source: HGNC Symbol; Acc: HGNC: 21839] | |
| ENSG00000247699 | | | 0.017813935 |
| ENSG00000248975 | | | 0.017824111 |
| ENSG00000179813 | FAM216B | family with sequence similarity 216 member B | 0.01797203 |
| | | [Source: HGNC Symbol; Acc: HGNC: 26883] | |
| ENSG00000188706 | ZDHHC9 | zinc finger DHHC-type containing 9 | 0.018020454 |
| | | [Source: HGNC Symbol: Acc: HGNC: 18475] | |
| ENSG00000135472 | FAIM2 | Fas apoptotic inhibitory molecule 2 | 0.018071818 |
| | | [Source: HGNC Symbol; Acc: HGNC: 17067] | |
| ENSG00000173572 | NLRP13 | NLR family pyrin domain containing 13 | 0.018071818 |
| | | [Source: HGNC Symbol; Acc: HGNC: 22937] | |
| ENSG00000089199 | CHGB | chromogranin B | 0.018179173 |
| | | [Source: HGNC Symbol; Acc: HGNC: 1930] | |
| ENSG00000188112 | C6orf132 | chromosome 6 open reading frame 132 | 0.018577564 |
| | | [Source: HGNC Symbol; Acc: HGNC: 21288] | |
| ENSG00000187068 | C3orf70 | chromosome 3 open reading frame 70 | 0.018587013 |
| | | [Source: HGNC Symbol; Acc: HGNC: 33731] | |
| ENSG00000233973 | LINC01360 | long intergenic non-protein coding RNA 1360 | 0.018588752 |
| | | [Source: HGNC Symbol; Acc: HGNC: 50593] | |
| ENSG00000164265 | SCGB3A2 | secretoglobin family 3 A member 2 | 0.018614288 |
| | | [Source: HGNC Symbol; Acc: HGNC: 18391] | |
| ENSG00000176769 | TCERG1L | transcription elongation regulator 1 like | 0.018783363 |
| | | [Source: HGNC Symbol; Acc: HGNC: 23533] | |
| ENSG00000179709 | NLRP8 | NLR family pyrin domain containing 8 | 0.018812736 |
| | | [Source: HGNC Symbol; Acc: HGNC: 22940] | |
| ENSG00000251557 | HNRNPKP3 | heterogeneous nuclear ribonucleoprotein K | 0.018866754 |
| | | pseudogene 3 | |
| | | [Source: HGNC Symbol; Acc: HGNC: 42376] | |
| ENSG00000149654 | CDH22 | cadherin 22 | 0.018978105 |
| | | [Source: HGNC Symbol; Acc: HGNC: 13251] | |
| ENSG00000170426 | SDR9C7 | short chain dehydrogenase/reductase family | 0.018978105 |
| | | 9C member 7 | |
| | | [Source: HGNC Symbol; Acc: HGNC: 29958] | |
| ENSG00000225637 | | | 0.019088297 |
| ENSG00000142408 | CACNG8 | calcium voltage-gated channel auxiliary subunit | 0.019108077 |
| | | gamma 8 | |
| | | [Source: HGNC Symbol; Acc: HGNC: 13628] | |
| ENSG00000230873 | STMND1 | stathmin domain containing 1 | 0.01920241 |
| | | [Source: HGNC Symbol; Acc: HGNC: 44668] | |
| ENSG00000236404 | VLDLR-AS1 | VLDLR antisense RNA 1 | 0.01920241 |
| | | [Source: HGNC Symbol; Acc: HGNC: 49621] | |
| ENSG00000170927 | PKHD1 | “PKHD1, fibrocystin/polyductin | 0.019345013 |
| | | [Source: HGNC Symbol; Acc: HGNC: 9016]” | |
| ENSG00000237289 | CKMT1B | “creatine kinase, mitochondrial 1B | 0.019345013 |
| | | [Source: HGNC Symbol; Acc: HGNC: 1995]” | |
| ENSG00000229817 | | | 0.019542531 |
| ENSG00000259176 | NA | NA | 0.019829586 |
| ENSG00000124092 | CTCFL | CCCTC-binding factor like | 0.019839425 |
| | | [Source: HGNC Symbol; Acc: HGNC: 16234] | |
| ENSG00000259156 | CHEK2P2 | checkpoint kinase 2 pseudogene 2 | 0.019859771 |
| | | [Source: HGNC Symbol; Acc: HGNC: 43578] | |
| ENSG00000203805 | PLPP4 | phospholipid phosphatase 4 | 0.019917239 |
| | | [Source: HGNC Symbol; Acc: HGNC: 23531] | |
| ENSG00000163914 | RHO | rhodopsin | 0.019927103 |
| | | [Source: HGNC Symbol; Acc: HGNC: 10012] | |
| ENSG00000224435 | NF1P6 | neurofibromin 1 pseudogene 6 | 0.019927103 |
| | | [Source: HGNC Symbol; Acc: HGNC: 7771] | |
| ENSG00000240707 | LINC01168 | long intergenic non-protein coding RNA 1168 | 0.019935944 |
| | | [Source: HGNC Symbol: Acc: HGNC: 49537] | |
| ENSG00000130045 | NXNL2 | nucleoredoxin like 2 | 0.020063533 |
| | | [Source: HGNC Symbol: Acc: HGNC: 30482] | |
| ENSG00000162062 | TEDC2 | tubulin epsilon and delta complex 2 | 0.020213465 |
| | | [Source: HGNC Symbol; Acc: HGNC: 25849] | |
| ENSG00000172752 | COL6A5 | collagen type VI alpha 5 chain | 0.020225168 |
| | | [Source: HGNC Symbol; Acc: HGNC: 26674] | |
| ENSG00000101871 | MID1 | midline 1 | 0.020247513 |
| | | [Source: HGNC Symbol; Acc: HGNC: 7095] | |
| ENSG00000137648 | TMPRSS4 | transmembrane serine protease 4 | 0.020387859 |
| | | [Source: HGNC Symbol; Acc: HGNC: 11878] | |
| ENSG00000166473 | PKD1L2 | polycystin 1 like 2 (gene/pseudogene) | 0.020387859 |
| | | [Source: HGNC Symbol; Acc: HGNC: 21715] | |
| ENSG00000257907 | EEF1A1P17 | eukaryotic translation elongation factor 1 alpha | 0.020486161 |
| | | 1 pseudogene 17 | |
| | | [Source: HGNC Symbol; Acc: HGNC: 37890] | |
| ENSG00000128917 | DLL4 | delta like canonical Notch ligand 4 | 0.020505106 |
| | | [Source: HGNC Symbol; Acc: HGNC: 2910] | |
| ENSG00000259380 | | | 0.020626924 |
| ENSG00000179766 | ATP8B5P | “ATPase phospholipid transporting 8B5, pseudogene | 0.02065324 |
| | | [Source: HGNC Symbol; Acc: HGNC: 27245]” | |
| ENSG00000204624 | DISP3 | dispatched RND transporter family member 3 | 0.02065324 |
| | | [Source: HGNC Symbol; Acc: HGNC: 29251] | |
| ENSG00000163689 | C3orf67 | chromosome 3 open reading frame 67 | 0.020743462 |
| | | [Source: HGNC Symbol; Acc: HGNC: 24763] | |
| ENSG00000132321 | IQCA1 | IQ motif containing with AAA domain 1 | 0.020807093 |
| | | [Source: HGNC Symbol; Acc: HGNC: 26195] | |
| ENSG00000249119 | MTND6P4 | mitochondrially encoded NADH: ubiquinone | 0.020807093 |
| | | oxidoreductase core subunit 6 pseudogene 4 | |
| | | [Source: HGNC Symbol; Acc: HGNC: 39467] | |
| ENSG00000019505 | SYT13 | synaptotagmin 13 | 0.020829887 |
| | | [Source: HGNC Symbol; Acc: HGNC: 14962] | |
| ENSG00000143469 | SYT14 | synaptotagmin 14 | 0.020885035 |
| | | [Source: HGNC Symbol; Acc: HGNC: 23143] | |
| ENSG00000196136 | SERPINA3 | serpin family A member 3 | 0.02099734 |
| | | [Source: HGNC Symbol; Acc: HGNC: 16] | |
| ENSG00000165816 | VWA2 | von Willebrand factor A domain containing 2 | 0.021329095 |
| | | [Source: HGNC Symbol; Acc: HGNC: 24709] | |
| ENSG00000183317 | EPHA10 | EPH receptor A10 | 0.021329095 |
| | | [Source: HGNC Symbol; Acc: HGNC: 19987] | |
| ENSG00000072041 | SLC6A15 | solute carrier family 6 member 15 | 0.021369466 |
| | | [Source: HGNC SymboliAcc: HGNC: 13621] | |
| ENSG00000009709 | PAX7 | paired box 7 | 0.021525887 |
| | | [Source: HGNC Symbol; Acc: HGNC: 8621] | |
| ENSG00000172350 | ABCG4 | ATP binding cassette subfamily G member 4 | 0.021525887 |
| | | [Source: HGNC SymboliAcc: HGNC: 13884] | |
| ENSG00000183876 | ARSI | arylsulfatase family member I | 0.021711339 |
| | | [Source: HGNC Symbol; Acc: HGNC: 32521] | |
| ENSG00000213934 | HBG1 | hemoglobin subunit gamma 1 | 0.02185053 |
| | | [Source: HGNC Symbol; Acc: HGNC: 4831] | |
| ENSG00000186526 | CYP4F8 | cytochrome P450 family 4 subfamily F member 8 | 0.021913633 |
| | | [Source: HGNC Symbol: Acc: HGNC: 2648] | |
| ENSG00000161940 | BCL6B | B cell CLL/lymphoma 6B | 0.021959359 |
| | | [Source: HGNC Symbol; Acc: HGNC: 1002] | |
| ENSG00000164093 | PITX2 | paired like homeodomain 2 | 0.02227554 |
| | | [Source: HGNC Symbol; Acc: HGNC: 9005] | |
| ENSG00000110786 | PTPN5 | “protein tyrosine phosphatase, non-receptor type 5 | 0.022304898 |
| | | [Source: HGNC Symbol; Acc: HGNC: 9657]” | |
| ENSG00000145642 | SHISAL2B | shisa like 2B | 0.022689972 |
| | | [Source: HGNC Symbol; Acc: HGNC: 34236] | |
| ENSG00000260411 | NA | NA | 0.022689972 |
| ENSG00000135409 | AMHR2 | anti-Mullerian hormone receptor type 2 | 0.022721606 |
| | | [Source: HGNC Symbol; Acc: HGNC: 465] | |
| ENSG00000259458 | | | 0.022776502 |
| ENSG00000068078 | FGFR3 | fibroblast growth factor receptor 3 | 0.022896021 |
| | | [Source: HGNC Symbol; Acc: HGNC: 3690] | |
| ENSG00000161243 | FBXO27 | F-box protein 27 | 0.023440646 |
| | | [Source: HGNC Symbol; Acc: HGNC: 18753] | |
| ENSG00000101004 | NENL | ninein like | 0.023637109 |
| | | [Source: HGNC Symbol; Acc: HGNC: 29163] | |
| ENSG00000121207 | LRAT | lecithin retinol acyltransferase | 0.023637109 |
| | | [Source: HGNC Symbol; Acc: HGNC: 6685] | |
| ENSG00000140527 | WDR93 | WD repeat domain 93 | 0.023652058 |
| | | [Source: HGNC Symbol; Acc: HGNC: 26924] | |
| ENSG00000236824 | BCYRN1 | brain cytoplasmic RNA 1 | 0.023652058 |
| | | [Source: HGNC SymboliAcc: HGNC: 1022] | |
| ENSG00000101203 | COL20A1 | collagen type XX alpha 1 chain | 0.023671204 |
| | | [Source: HGNC SymboliAcc: HGNC: 14670] | |
| ENSG00000233977 | | | 0.023671204 |
| ENSG00000148408 | CACNA1B | calcium voltage-gated channel subunit alpha1 B | 0.02389629 |
| | | [Source: HGNC Symbol; Acc: HGNC: 1389] | |
| ENSG00000134240 | EEMGCS2 | 3-hydroxy-3-methylglutaryl-CoA synthase 2 | 0.023926586 |
| | | [Source: HGNC Symbol; Acc: HGNC: 5008] | |
| ENSG00000112186 | CAP2 | cyclase associated actin cytoskeleton regulatory | 0.024121501 |
| | | protein 2 | |
| | | [Source: HGNC Symbol; Acc: HGNC: 20039] | |
| ENSG00000182256 | GABRG3 | gamma-aminobutyric acid type A receptor | 0.024155164 |
| | | gamma3 subunit | |
| | | [Source: HGNC Symbol; Acc: HGNC: 4088] | |
| ENSG00000166159 | LRTM2 | leucine rich repeats and transmembrane domains 2 | 0.024214399 |
| | | [Source: HGNC Symbol; Acc: HGNC: 32443] | |
| ENSG00000132972 | RNF17 | ring finger protein 17 | 0.024283188 |
| | | [Source: HGNC Symbol; Acc: HGNC: 10060] | |
| ENSG00000156076 | WIF1 | WNT inhibitory factor 1 | 0.024283188 |
| | | [Source: HGNC Symbol; Acc: HGNC: 18081] | |
| ENSG00000261649 | GOLGA6L7 | golgin A6 family like 7 | 0.024421599 |
| | | [Source: HGNC Symbol; Acc: HGNC: 37442] | |
| ENSG00000112238 | PRDM13 | PR/SET domain 13 | 0.02443315 |
| | | [Source: HGNC Symbol; Acc: HGNC: 13998] | |
| ENSG00000166391 | MOGAT2 | monoacylglycerol O-acyltransferase 2 | 0.024463522 |
| | | [Source: HGNC Symbol; Acc: HGNC: 23248] | |
| ENSG00000166869 | CHP2 | calcineurin like EF-hand protein 2 | 0.024463522 |
| | | [Source: HGNC Symbol; Acc: HGNC: 24927] | |
| ENSG00000218823 | PAPOLB | poly(A) polymerase beta | 0.024463522 |
| | | [Source: HGNC Symbol; Acc: HGNC: 15970] | |
| ENSG00000265041 | | | 0.024463522 |
| ENSG00000133124 | IRS4 | insulin receptor substrate 4 | 0.024526611 |
| | | [Source: HGNC Symbol; Acc: HGNC: 6128] | |
| ENSG00000118733 | OLFM3 | olfactomedin 3 | 0.024577949 |
| | | [Source: HGNC Symbol; Acc: HGNC: 17990] | |
| ENSG00000196091 | MYBPC1 | “myosin binding protein C, slow type | 0.024577949 |
| | | [Source: HGNC Symbol; Acc: HGNC: 7549]” | |
| ENSG00000105357 | MYH14 | myosin heavy chain 14 | 0.025148395 |
| | | [Source: HGNC Symbol; Acc: HGNC: 23212] | |
| ENSG00000167757 | KLK11 | kallikrein related peptidase 11 | 0.025148395 |
| | | [Source: HGNC Symbol; Acc: HGNC: 6359] | |
| ENSG00000226068 | HNRNPA3P4 | heterogeneous nuclear ribonucleoprotein A3 | 0.025148395 |
| | | pseudogene 4 | |
| | | [Source: HGNC Symbol; Acc: HGNC: 39773] | |
| ENSG00000260072 | | | 0.025148395 |
| ENSG00000130226 | DPP6 | dipeptidyl peptidase like 6 | 0.02515224 |
| | | [Source: HGNC Symbol; Acc: HGNC: 3010] | |
| ENSG00000144648 | ACKR2 | atypical chemokine receptor 2 | 0.02527518 |
| | | [Source: HGNC Symbol; Acc: HGNC: 1565] | |
| ENSG00000169862 | CTNND2 | catenin delta 2 | 0.025318552 |
| | | [Source: HGNC Symbol; Acc: HGNC: 2516] | |
| ENSG00000137766 | UNC13C | unc-13 homolog C | 0.025345898 |
| | | [Source: HGNC Symbol; Acc: HGNC: 23149] | |
| ENSG00000261177 | | | 0.025565995 |
| ENSG00000060656 | PTPRU | “protein tyrosine phosphatase, receptor type U | 0.025652607 |
| | | [Source: HGNC Symbol; Acc: HGNC: 9683]” | |
| ENSG00000260305 | NTRK3-AS1 | NTRK3 antisense RNA 1 | 0.02580767 |
| | | [Source: HGNC Symbol; Acc: HGNC: 27532] | |
| ENSG00000187955 | COL14A1 | collagen type XIV alpha 1 chain | 0.025820696 |
| | | [Source: HGNC Symbol; Acc: HGNC: 2191] | |
| ENSG00000089225 | TBX5 | T-box 5 | 0.025834296 |
| | | [Source: HGNC Symbol; Acc: HGNC: 11604] | |
| ENSG00000224209 | LINC00466 | long intergenic non-protein coding RNA 466 | 0.025987072 |
| | | [Source: HGNC Symbol; Acc: HGNC: 27294] | |
| ENSG00000151474 | FRMD4A | FERM domain containing 4A | 0.026041989 |
| | | [Source: HGNC Symbol; Acc: HGNC: 25491] | |
| ENSG00000039987 | BEST2 | bestrophin 2 | 0.026152714 |
| | | [Source: HGNC Symbol; Acc: HGNC: 17107] | |
| ENSG00000266795 | NA | NA | 0.026198035 |
| ENSG00000181143 | MUC16 | “mucin 16, cell surface associated | 0.026247081 |
| | | [Source: HGNC Symbol; Acc: HGNC: 15582]” | |
| ENSG00000069431 | ABCC9 | ATP binding cassette subfamily C member 9 | 0.026486685 |
| | | [Source: HGNC Symbol; Acc: HGNC: 60] | |
| ENSG00000100312 | ACR | acrosin | 0.02666392 |
| | | [Source: HGNC Symbol; Acc: HGNC: 126] | |
| ENSG00000254042 | | | 0.026721536 |
| ENSG00000180251 | SLC9A4 | solute carrier family 9 member A4 | 0.026759131 |
| | | [Source: HGNC Symbol; Acc: HGNC: 11077] | |
| ENSG00000237390 | | | 0.026759131 |
| ENSG00000246695 | RASSF8-AS1 | RASSF8 antisense RNA 1 | 0.026759131 |
| | | [Source: HGNC Symbol; Acc: HGNC: 48637] | |
| ENSG00000256612 | CYP2B7P | “cytochrome P450 family 2 subfamily B member | 0.026759131 |
| | | 7, pseudogene | |
| | | [Source: HGNC Symbol; Acc: HGNC: 2616]” | |
| ENSG00000165973 | NELL1 | neural EGFL like 1 | 0.026774963 |
| | | [Source: HGNC Symbol; Acc: HGNC: 7750] | |
| ENSG00000172900 | | | 0.02698221 |
| ENSG00000149926 | FAM57B | family with sequence similarity 57 member B | 0.02707614 |
| | | [Source: HGNC Symbol; Acc: HGNC: 25295] | |
| ENSG00000107295 | SH3GL2 | “SH3 domain containing GRB2 like 2, endophilin A1 | 0.027164347 |
| | | [Source: HGNC Symbol· Acc: HGNC: 10831]” | |
| ENSG00000173227 | SYT12 | synaptotagmin 12 | 0.027164347 |
| | | [Source: HGNC Symbol; Acc: HGNC: 18381] | |
| ENSG00000173013 | CCDC96 | coiled-coil domain containing 96 | 0.027208218 |
| | | [Source: HGNC Symbol; Acc: HGNC: 26900] | |
| ENSG00000268460 | | | 0.02723306 |
| ENSG00000234512 | TLR12P | “toll like receptor 12, pseudogene | 0.027406947 |
| | | [Source: HGNC Symbol; Acc: HGNC: 31754]” | |
| ENSG00000135931 | ARMC9 | armadillo repeat containing 9 | 0.02759323 |
| | | [Source: HGNC Symbol; Acc: HGNC: 20730] | |
| ENSG00000148702 | HABP2 | hyaluronan binding protein 2 | 0.027601758 |
| | | [Source: HGNC Symbol; Acc: HGNC: 4798] | |
| ENSG00000136535 | TBR1 | “T-box, brain 1 | 0.028071412 |
| | | [Source: HGNC Symbol; Acc: HGNC: 11590]” | |
| ENSG00000122121 | XPNPEP2 | X-prolyl aminopeptidase 2 | 0.028133383 |
| | | [Source: HGNC Symbol; Acc: HGNC: 12823] | |
| ENSG00000170442 | KRT86 | keratin 86 | 0.028133383 |
| | | [Source: HGNC Symbol; Acc: HGNC: 6463] | |
| ENSG00000197408 | CYP2B6 | cytochrome P450 family 2 subfamily B member 6 | 0.028133383 |
| | | [Source: HGNC Symbol; Acc: HGNC: 2615] | |
| ENSG00000107807 | TLX1 | T cell leukemia homeobox 1 | 0.028207054 |
| | | [Source: HGNC Symbol; Acc: HGNC: 5056] | |
| ENSG00000164694 | FNDC1 | fibronectin type III domain containing 1 | 0.028207054 |
| | | [Source: HGNC Symbol; Acc: HGNC: 21184] | |
| ENSG00000185313 | SCN10A | sodium voltage-gated channel alpha subunit 10 | 0.028207054 |
| | | [Source: HGNC Symbol; Acc: HGNC: 10582] | |
| ENSG00000164107 | HAND2 | heart and neural crest derivatives expressed 2 | 0.028335907 |
| | | [Source: HGNC Symbol; Acc: HGNC: 4808] | |
| ENSG00000133454 | MYO18B | myosin XVIIIB | 0.028417113 |
| | | [Source: HGNC Symbol; Acc: HGNC: 18150] | |
| ENSG00000167723 | TRPV3 | transient receptor potential cation channel | 0.028422765 |
| | | subfamily V member 3 | |
| | | [Source: HGNC Symbol: Acc: HGNC: 18084] | |
| ENSG00000184012 | TMPRSS2 | transmembrane serine protease 2 | 0.028422765 |
| | | [Source: HGNC Symbol; Acc: HGNC: 11876] | |
| ENSG00000233485 | | | 0.028645846 |
| ENSG00000261466 | | | 0.028645846 |
| ENSG00000119547 | ONECUT2 | one cut homeobox 2 | 0.028669408 |
| | | [Source: HGNC Symbol; Acc: HGNC: 8139] | |
| ENSG00000237222 | LINC01968 | long intergenic non-protein coding RNA 1968 | 0.028800664 |
| | | [Source: HGNC Symbol; Acc: HGNC: 52794] | |
| ENSG00000137573 | SULF1 | sulfatase 1 | 0.028919683 |
| | | [Source: HGNC Symbol; Acc: HGNC: 20391] | |
| ENSG00000161609 | CCDC155 | coiled-coil domain containing 155 | 0.028967146 |
| | | [Source: HGNC Symbol; Acc: HGNC: 26520] | |
| ENSG00000250546 | | | 0.028987628 |
| ENSG00000226057 | PHF2P2 | PHD finger protein 2 pseudogene 2 | 0.029139308 |
| | | [Source: HGNC Symbol; Acc: HGNC: 38808] | |
| ENSG00000177045 | SIX5 | SIX homeobox 5 | 0.029298722 |
| | | [Source: HGNC Symbol; Acc: HGNC: 10891] | |
| ENSG00000124440 | HIF3A | hypoxia inducible factor 3 alpha subunit | 0.029322985 |
| | | [Source: HGNC Symbol; Acc: HGNC: 15825] | |
| ENSG00000234828 | IQCM | IQ motif containing M | 0.029461236 |
| | | [Source: HGNC Symbol; Acc: HGNC: 53443] | |
| ENSG00000116721 | PRAMEF1 | PRAME family member 1 | 0.029473652 |
| | | [Source: HGNC Symbol; Acc: HGNC: 28840] | |
| ENSG00000238116 | | | 0.029473652 |
| ENSG00000106689 | LHX2 | LIM homeobox 2 | 0.029512187 |
| | | [Source: HGNC Symbol; Acc: HGNC: 6594] | |
| ENSG00000169344 | UMOD | uromodulin | 0.02959944 |
| | | [Source: HGNC Symbol; Acc: HGNC: 12559] | |
| ENSG00000174279 | EVX2 | even-skipped homeobox 2 | 0.029661965 |
| | | [Source: HGNC Symbol; Acc: HGNC: 3507] | |
| ENSG00000128573 | FOXP2 | forkhead box P2 | 0.029779428 |
| | | [Source: HGNC Symbol; Acc: HGNC: 13875] | |
| ENSG00000251596 | HADHAP1 | “hydroxyacyl-CoA dehydrogenase/3-ketoacyl-CoA | 0.029954508 |
| | | thiolase/enoyl-CoA hydratase (trifunctional protein), | |
| | | alpha subunit pseudogene | |
| | | 1[Source: HGNC Symbol; Acc: HGNC: 4802]” | |
| ENSG00000002746 | HECW1 | “HECT, C2 and WW domain containing E3 | 0.029980732 |
| | | ubiquitin protein ligase 1 | |
| | | [Source: HGNC Symbol; Acc: HGNC: 22195]” | |
| ENSG00000081248 | CACNA1S | calcium voltage-gated channel subunit alpha1 S | 0.029997906 |
| | | [Source: HGNC Symbol; Acc: HGNC: 1397] | |
| ENSG00000166596 | CFAP52 | cilia and flagella associated protein 52 | 0.030027148 |
| | | [Source: HGNC Symbol; Acc: HGNC: 16053] | |
| ENSG00000205176 | REXO1L1P | “REXO1 like 1, pseudogene | 0.030066221 |
| | | [Source: HGNC Symbol; Acc: HGNC: 24660]” | |
| ENSG00000152910 | CNTNAP4 | contactin associated protein like 4 | 0.030144659 |
| | | [Source: HGNC Symbol; Acc: HGNC: 18747] | |
| ENSG00000106078 | COBL | cordon-bleu WH2 repeat protein | 0.030263618 |
| | | [Source: HGNC Symbol; Acc: HGNC: 22199] | |
| ENSG00000177103 | DSCAML1 | DS cell adhesion molecule like 1 | 0.030299369 |
| | | [Source: HGNC Symbol; Acc: HGNC: 14656] | |
| ENSG00000131044 | TTLL9 | tubulin tyrosine ligase like 9 | 0.030317293 |
| | | [Source: HGNC Symbol; Acc: HGNC: 16118] | |
| ENSG00000170703 | TTLL6 | tubulin tyrosine ligase like 6 | 0.030472844 |
| | | [Source: HGNC Symbol; Acc: HGNC: 26664] | |
| ENSG00000165379 | LRFN5 | leucine rich repeat and fibronectin type III | 0.030532839 |
| | | domain containing 5 | |
| | | [Source: HGNC Symbol; Acc: HGNC: 20360] | |
| ENSG00000198929 | NOS1AP | nitric oxide synthase 1 adaptor protein | 0.030532839 |
| | | [Source: HGNC Symbol; Acc: HGNC: 16859] | |
| ENSG00000236253 | SLC25A3P1 | solute carrier family 25 member 3 pseudogene 1 | 0.030574302 |
| | | [Source: HGNC Symbol; Acc: HGNC: 26869] | |
| ENSG00000205667 | ARSH | arylsulfatase family member H | 0.030639161 |
| | | [Source: HGNC Symbol; Acc: HGNC: 32488] | |
| ENSG00000226440 | | | 0.030639161 |
| ENSG00000131183 | SLC34A1 | solute carrier family 34 member 1 | 0.030803937 |
| | | [Source: HGNC Symbol; Acc: HGNC: 11019] | |
| ENSG00000225649 | | | 0.030847037 |
| ENSG00000006283 | CACNA1G | calcium voltage-gated channel subunit alpha1 G | 0.030936635 |
| | | [Source: HGNC Symbol; Acc: HGNC: 1394] | |
| ENSG00000230392 | | | 0.030977697 |
| ENSG00000234130 | | | 0.031067495 |
| ENSG00000095637 | SORBS1 | sorbin and SH3 domain containing 1 | 0.031086939 |
| | | [Source: HGNC Symbol; Acc: HGNC: 14565] | |
| ENSG00000198010 | DLGAP2 | DLG associated protein 2 | 0.031235492 |
| | | [Source: HGNC Symbol; Acc: HGNC: 2906] | |
| ENSG00000102290 | PCDH11X | protocadherin 11 X-linked | 0.031415941 |
| | | [Source: HGNC Symbol; Acc: HGNC: 8656] | |
| ENSG00000260027 | HOXB7 | homeobox B7 | 0.031452441 |
| | | [Source: HGNC Symbol; Acc: HGNC: 5118] | |
| ENSG00000105664 | COMP | cartilage oligomeric matrix protein | 0.031516506 |
| | | [Source: HGNC Symbol; Acc: HGNC: 2227] | |
| ENSG00000006071 | ABCC8 | ATP binding cassette subfamily C member 8 | 0.031578145 |
| | | [Source: HGNC Symbol; Acc: HGNC: 59] | |
| ENSG00000077522 | ACTN2 | actinin alpha 2 | 0.031657927 |
| | | [Source: HGNC Symbol; Acc: HGNC: 164] | |
| ENSG00000248966 | BCLAF1P1 | BCL2 associated transcription factor 1 pseudogene 1 | 0.031733491 |
| | | [Source: HGNC Symbol; Acc: HGNC: 51329] | |
| ENSG00000124749 | COL21A1 | collagen type XXI alpha 1 chain | 0.031814031 |
| | | [Source: HGNC Symbol; Acc: HGNC: 17025] | |
| ENSG00000142675 | CNKSR1 | connector enhancer of kinase suppressor of Ras 1 | 0.031815396 |
| | | [Source: HGNC Symbol; Acc: HGNC: 19700] | |
| ENSG00000116748 | AMPD1 | adenosine monophosphate deaminase 1 | 0.031850457 |
| | | [Source: HGNC Symbol; Acc: HGNC: 468] | |
| ENSG00000181355 | OFCC1 | orofacial cleft 1 candidate 1 | 0.03221007 |
| | | [Source: HGNC Symbol; Acc: HGNC: 21017] | |
| ENSG00000162510 | MATN1 | “matrilin 1, cartilage matrix protein | 0.032424937 |
| | | [Source: HGNC Symbol; Acc: HGNC: 6907]” | |
| ENSG00000232392 | | | 0.032424937 |
| ENSG00000123572 | NRK | Nik related kinase | 0.032537919 |
| | | [Source: HGNC Symbol; Acc: HGNC: 25391] | |
| ENSG00000267324 | | | 0.032537919 |
| ENSG00000196361 | ELAVL3 | ELAV like RNA binding protein 3 | 0.032579843 |
| | | [Source: HGNC Symbol; Acc: HGNC: 3314] | |
| ENSG00000204661 | C5orf60 | chromosome 5 open reading frame 60 | 0.032697662 |
| | | [Source: HGNC Symbol; Acc: HGNC: 27753] | |
| ENSG00000224059 | HSPA8P16 | heat shock protein family A (Hsp70) member | 0.03275969 |
| | | 8 pseudogene 16 | |
| | | [Source: HGNC Symbol; Acc: HGNC: 44931] | |
| ENSG00000114019 | AMOTL2 | angiomotin like 2 | 0.033101928 |
| | | [Source: HGNC Symbol; Acc: HGNC: 17812] | |
| ENSG00000134871 | COL4A2 | collagen type IV alpha 2 chain | 0.033101928 |
| | | [Source: HGNC Symbol; Acc: HGNC: 2203] | |
| ENSG00000162706 | CADM3 | cell adhesion molecule 3 | 0.033101928 |
| | | [Source: HGNC Symbol; Acc: HGNC: 17601] | |
| ENSG00000188782 | CATSPER4 | cation channel sperm associated 4 | 0.033196706 |
| | | [Source: HGNC Symbol; Acc: HGNC: 23220] | |
| ENSG00000147689 | FAM83A | family with sequence similarity 83 member A | 0.033349502 |
| | | [Source: HGNC Symbol; Acc: HGNC: 28210] | |
| ENSG00000079841 | RIMS1 | regulating synaptic membrane exocytosis 1 | 0.033394348 |
| | | [Source: HGNC Symbol; Acc: HGNC: 17282] | |
| ENSG00000103647 | CORO2B | coronin 2B | 0.033419797 |
| | | [Source: HGNC Symbol; Acc: HGNC: 2256] | |
| ENSG00000112499 | SLC22A2 | solute carrier family 22 member 2 | 0.033434322 |
| | | [Source: HGNC Symbol; Acc: HGNC: 10966] | |
| ENSG00000183856 | IQGAP3 | IQ motif containing GTPase activating protein 3 | 0.033434322 |
| | | [Source: HGNC Symbol; Acc: HGNC: 20669] | |
| ENSG00000165300 | SLITRK5 | SLIT and NTRK like family member 5 | 0.033483825 |
| | | [Source: HGNC Symbol; Acc: HGNC: 20295] | |
| ENSG00000229972 | IQCF3 | IQ motif containing F3 | 0.033483825 |
| | | [Source: HGNC Symbol; Acc: HGNC: 31816] | |
| ENSG00000261949 | GFY | golgi associated olfactory signaling regulator | 0.033483825 |
| | | [Source: HGNC Symbol; Acc: HGNC: 44663] | |
| ENSG00000171487 | NLRP5 | NLR family pyrin domain containing 5 | 0.033631095 |
| | | [Source: HGNC Symbol; Acc: HGNC: 21269] | |
| ENSG00000129946 | SHC2 | SHC adaptor protein 2 | 0.033699292 |
| | | [Source: HGNC Symbol; Acc: HGNC: 29869] | |
| ENSG00000117501 | MROH9 | maestro heat like repeat family member 9 | 0.03391477 |
| | | [Source: HGNC Symbol; Acc: HGNC: 26287] | |
| ENSG00000136574 | GATA4 | GATA binding protein 4 | 0.034539616 |
| | | [Source: HGNC Symbol; Acc: HGNC: 4173] | |
| ENSG00000106648 | GALNTL5 | polypeptide N-acetylgalactosaminyltransferase like 5 | 0.034620414 |
| | | Source: HGNC Symbol; Acc: HGNC: 21725] | |
| ENSG00000188086 | PRSS45 | serine protease 45 | 0.034840251 |
| | | [Source: HGNC Symbol; Acc: HGNC: 30717] | |
| ENSG00000234537 | | | 0.034858474 |
| ENSG00000226741 | LINC02554 | long intergenic non-protein coding RNA 2554 | 0.034969314 |
| | | [Source: HGNC Symbol; Acc: HGNC: 53594] | |
| ENSG00000004948 | CALCR | calcitonin receptor | 0.034980702 |
| | | [Source: HGNC Symbol; Acc: HGNC: 1440] | |
| ENSG00000142549 | IGLON5 | IgLON family member 5 | 0.034980702 |
| | | [Source: HGNC Symbol; Acc: HGNC: 34550] | |
| ENSG00000250519 | | | 0.034980702 |
| ENSG00000183908 | LRRC55 | leucine rich repeat containing 55 | 0.035005257 |
| | | [Source: HGNC Symbol; Acc: HGNC: 32324] | |
| ENSG00000253974 | NRG1-IT1 | NRG1 intronic transcript 1 | 0.035154264 |
| | | [Source: HGNC Symbol; Acc: HGNC: 43633] | |
| ENSG00000162738 | VANGL2 | VANGL planar cell polarity protein 2 | 0.035338561 |
| | | [Source: HGNC Symbol; Acc: HGNC: 15511] | |
| ENSG00000115648 | MLPH | melanophilin | 0.03575577 |
| | | [Source: HGNC Symbol; Acc: HGNC: 29643] | |
| ENSG00000187997 | C17orf99 | chromosome 17 open reading frame 99 | 0.03575577 |
| | | [Source: HGNC Symbol; Acc: HGNC: 34490] | |
| ENSG00000140279 | DUOX2 | dual oxidase 2 | 0.035790036 |
| | | [Source: HGNC Symbol; Acc: HGNC: 13273] | |
| ENSG00000168077 | SCARA3 | scavenger receptor class A member 3 | 0.035804565 |
| | | [Source: HGNC Symbol; Acc: HGNC: 19000] | |
| ENSG00000159337 | PLA2G4D | phospholipase A2 group IVD | 0.035823277 |
| | | [Source: HGNC Symbol; Acc: HGNC: 30038] | |
| ENSG00000183580 | FBXL7 | F-box and leucine rich repeat protein 7 | 0.035823277 |
| | | [Source: HGNC Symbol; Acc: HGNC: 13604] | |
| ENSG00000218586 | | | 0.035823277 |
| ENSG00000184809 | B3GALT5-AS1 | B3GALT5 antisense RNA 1 | 0.035845893 |
| | | [Source: HGNC Symbol; Acc: HGNC: 16424] | |
| ENSG00000132975 | GPR12 | G protein-coupled receptor 12 | 0.035938935 |
| | | [Source: HGNC Symbol; Acc: HGNC: 4466] | |
| ENSG00000142910 | TINAGL1 | tubulointerstitial nephritis antigen like 1 | 0.035938935 |
| | | [Source: HGNC Symbol; Acc: HGNC: 19168] | |
| ENSG00000075891 | PAX2 | paired box 2 | 0.035996581 |
| | | [Source: HGNC Symbol; Acc: HGNC: 8616] | |
| ENSG00000186393 | KRT26 | keratin 26 | 0.036025366 |
| | | [Source: HGNC Symbol; Acc: HGNC: 30840] | |
| ENSG00000167779 | IGFBP6 | insulin like growth factor binding protein 6 | 0.036065767 |
| | | [Source: HGNC Symbol; Acc: HGNC: 5475] | |
| ENSG00000232667 | | | 0.036065767 |
| ENSG00000263711 | | | 0.036105875 |
| ENSG00000205054 | LINC01121 | long intergenic non-protein coding RNA 1121 | 0.036340531 |
| | | [Source: HGNC Symbol; Acc: HGNC: 49266] | |
| ENSG00000146950 | SHROOM2 | shroom family member 2 | 0.036393162 |
| | | [Source: HGNC Symbol; Acc: HGNC: 630] | |
| ENSG00000143867 | OSR1 | odd-skipped related transciption factor 1 | 0.036631586 |
| | | [Source: HGNC Symbol; Acc: HGNC: 8111] | |
| ENSG00000205976 | | | 0.037140956 |
| ENSG00000196862 | RGPD4 | RANBP2-like and GRIP domain containing 4 | 0.037154755 |
| | | [Source: HGNC Symbol; Acc: HGNC: 32417] | |
| ENSG00000148513 | ANKRD30A | ankyrin repeat domain 30A | 0.037278195 |
| | | [Source: HGNC Symbol; Acc: HGNC: 17234] | |
| ENSG00000101057 | MYBL2 | MYB proto-oncogene like 2 | 0.037361359 |
| | | [Source: HGNC Symbol; Acc: HGNC: 7548] | |
| ENSG00000139144 | PIK3C2G | phosphatidylinositol-4-phosphate 3-kinase | 0.037546969 |
| | | catalytic subunit type 2 | |
| | | Gamma[Source: HGNC Symbol; Acc: HGNC: 8973] | |
| ENSG00000247213 | LINC01498 | long intergenic non-protein coding RNA 1498 | 0.037546969 |
| | | [Source: HGNC Symbol; Acc: HGNC: 51164] | |
| ENSG00000145242 | EPHA5 | EPH receptor A5 | 0.037630556 |
| | | [Source: HGNC Symbol; Acc: HGNC: 3389] | |
| ENSG00000249215 | NCOA4P4 | nuclear receptor coactivator 4 pseudogene 4 | 0.037740576 |
| | | [Source: HGNC Symbol; Acc: HGNC: 52405] | |
| ENSG00000079112 | CDH17 | cadherin 17 | 0.037745905 |
| | | [Source: HGNC Symbol; Acc: HGNC: 1756] | |
| ENSG00000166118 | SPATA19 | spermatogenesis associated 19 | 0.037802718 |
| | | [Source: HGNC Symbol; Acc: HGNC: 30614] | |
| ENSG00000162006 | MSLNL | mesothelin-like | 0.037970738 |
| | | [Source: HGNC Symbol; Acc: HGNC: 14170] | |
| ENSG00000187123 | LYPD6 | LY6/PLAUR domain containing 6 | 0.037980544 |
| | | [Source: HGNC Symbol: Acc: HGNC: 28751] | |
| ENSG00000104313 | EYA1 | EYA transcriptional coactivator and phosphatase 1 | 0.038059131 |
| | | [Source: HGNC Symbol; Acc: HGNC: 3519] | |
| ENSG00000237250 | | | 0.038171217 |
| ENSG00000105290 | APLP1 | amyloid beta precursor like protein 1 | 0.038576481 |
| | | [Source: HGNC Symbol; Acc: HGNC: 597] | |
| ENSG00000138650 | PCDH10 | protocadherin 10 | 0.038631087 |
| | | [Source: HGNC Symbol; Acc: HGNC: 13404] | |
| ENSG00000198914 | POU3F3 | POU class 3 homeobox 3 | 0.03865691 |
| | | [Source: HGNC Symbol; Acc: HGNC: 9216] | |
| ENSG00000117114 | ADGRL2 | adhesion G protein-coupled receptor L2 | 0.039101789 |
| | | [Source: HGNC Symbol; Acc: HGNC: 18582] | |
| ENSG00000185737 | NRG3 | neuregulin 3 | 0.039101789 |
| | | [Source: HGNC Symbol; Acc: HGNC: 7999] | |
| ENSG00000197085 | NPSR1-AS1 | NPSR1 antisense RNA 1 | 0.039361047 |
| | | [Source: HGNC Symbol; Acc: HGNC: 22128] | |
| ENSG00000230102 | LINC02028 | long intergenic non-protein coding RNA 2028 | 0.039602594 |
| | | [Source: HGNC Symbol; Acc: HGNC: 27718] | |
| ENSG00000241158 | ADAMTS9-AS1 | ADAMTS9 antisense RNA 1 | 0.039646513 |
| | | [Source: HGNC Symbol; Acc: HGNC: 40625] | |
| ENSG00000146005 | PSD2 | pleckstrin and Sec7 domain containing 2 | 0.039852243 |
| | | [Source: HGNC Symbol; Acc: HGNC: 19092] | |
| ENSG00000171533 | MAP6 | microtubule associated protein 6 | 0.040171006 |
| | | [Source: HGNC Symbol; Acc: HGNC: 6868] | |
| ENSG00000164294 | GPX8 | glutathione peroxidase 8 (putative) | 0.040207131 |
| | | [Source: HGNC Symbol; Acc: HGNC: 33100] | |
| ENSG00000054356 | PTPRN | “protein tyrosine phosphatase, receptor type N | 0.040248543 |
| | | [Source: HGNC Symbol; Acc: HGNC: 9676]” | |
| ENSG00000077080 | ACTL6B | actin like 6B | 0.040248543 |
| | | [Source: HGNC Symbol; Acc: HGNC: 160] | |
| ENSG00000141434 | MEP1B | meprin A subunit beta | 0.040590193 |
| | | [Source: HGNC Symbol; Acc: HGNC: 7020] | |
| ENSG00000183067 | IGSF5 | immunoglobulin superfamily member 5 | 0.040590193 |
| | | [Source: HGNC Symbol; Acc: HGNC: 5952] | |
| ENSG00000112337 | SLC17A2 | solute carrier family 17 member 2 | 0.040803316 |
| | | [Source: HGNC Symbol; Acc: HGNC: 10930] | |
| ENSG00000161682 | FAM171A2 | family with sequence similarity 171 member A2 | 0.040923418 |
| | | [Source: HGNC Symbol; Acc: HGNC: 30480] | |
| ENSG00000116833 | NR5A2 | nuclear receptor subfamily 5 group A member 2 | 0.040938395 |
| | | [Source: HGNC Symbol; Acc: HGNC: 7984] | |
| ENSG00000143355 | LHX9 | LIM homeobox 9 | 0.041155655 |
| | | [Source: HGNC Symbol; Acc: HGNC: 14222] | |
| ENSG00000139767 | SRRM4 | serine/arginine repetitive matrix 4 | 0.041207854 |
| | | [Source: HGNC Symbol; Acc: HGNC: 29389] | |
| ENSG00000227036 | LINC00511 | long intergenic non-protein coding RNA 511 | 0.041207854 |
| | | [Source: HGNC Symbol; Acc: HGNC: 43564] | |
| ENSG00000105549 | THEG | theg spermatid protein | 0.041581527 |
| | | [Source: HGNC Symbol; Acc: HGNC: 13706] | |
| ENSG00000104967 | NOVA2 | NOVA alternative splicing regulator 2 | 0.041600384 |
| | | [Source: HGNC Symbol; Acc: HGNC: 7887] | |
| ENSG00000183206 | POTEC | POTE ankyrin domain family member C | 0.041620804 |
| | | [Source: HGNC Symbol; Acc: HGNC: 33894] | |
| ENSG00000184302 | SIX6 | SIX homeobox 6 | 0.041631474 |
| | | [Source: HGNC Symbol; Acc: HGNC: 10892] | |
| ENSG00000245651 | | | 0.041631474 |
| ENSG00000179178 | TMEM125 | transmembrane protein 125 | 0.041791867 |
| | | [Source: HGNC Symbol; Acc: HGNC: 28275] | |
| ENSG00000231422 | LINC01516 | long intergenic non-protein coding RNA 1516 | 0.041868067 |
| | | [Source: HGNC Symbol; Acc: HGNC: 51211] | |
| ENSG00000104435 | STMN2 | stathmin 2 | 0.041944166 |
| | | [Source: HGNC Symbol; Acc: HGNC: 10577] | |
| ENSG00000185069 | KRT76 | keratin 76 | 0.042071807 |
| | | [Source: HGNC Symbol; Acc: HGNC: 24430] | |
| ENSG00000060709 | RIMBP2 | RIMS binding protein 2 | 0.042101327 |
| | | [Source: HGNC Symbol; Acc: HGNC: 30339] | |
| ENSG00000261115 | TMEM178B | transmembrane protein 178B | 0.042193233 |
| | | [Source: HGNC Symbol; Acc: HGNC: 44112] | |
| ENSG00000261623 | LINC02179 | long intergenic non-protein coding RNA 2179 | 0.042224694 |
| | | [Source: HGNC Symbol; Acc: HGNC: 53041] | |
| ENSG00000153165 | RGPD3 | RANBP2-like and GRIP domain containing 3 | 0.042347063 |
| | | [Source: HGNC Symbol; Acc: HGNC: 32416] | |
| ENSG00000253230 | LINC00599 | long intergenic non-protein coding RNA 599 | 0.042450091 |
| | | [Source: HGNC Symbol; Acc: HGNC: 27231] | |
| ENSG00000236078 | LINC01447 | long intergenic non-protein coding RNA 1447 | 0.042463159 |
| | | [Source: HGNC Symbol; Acc: HGNC: 50783] | |
| ENSG00000230133 | LINC01721 | long intergenic non-protein coding RNA 1721 | 0.042512831 |
| | | [Source: HGNC Symbol; Acc: HGNC: 52508] | |
| ENSG00000237636 | ANKRD26P3 | ankyrin repeat domain 26 pseudogene 3 | 0.042582368 |
| | | [Source: HGNC Symbol; Acc: HGNC: 39689] | |
| ENSG00000264954 | PRR29-AS1 | PRR29 antisense RNA 1 | 0.042699245 |
| | | [Source: HGNC Symbol; Acc: HGNC: 51822] | |
| ENSG00000166689 | PLEKHA7 | pleckstrin homology domain containing A7 | 0.04272194 |
| | | [Source: HGNC Symbol; Acc: HGNC: 27049] | |
| ENSG00000173826 | KCNH6 | potassium voltage-gated channel subfamily H | 0.042801591 |
| | | member 6 | |
| | | [Source: HGNC Symbol; Acc: HGNC: 18862] | |
| ENSG00000253864 | NA | NA | 0.042900836 |
| ENSG00000166292 | TMEM100 | transmembrane protein 100 | 0.043052047 |
| | | [Source: HGNC Symbol; Acc: HGNC: 25607] | |
| ENSG00000137203 | TFAP2A | transcription factor AP-2 alpha | 0.043105155 |
| | | [Source: HGNC Symbol; Acc: HGNC: 11742] | |
| ENSG00000165970 | SLC6A5 | solute carrier family 6 member 5 | 0.043105155 |
| | | [Source: HGNC SymboliAcc: HGNC: 11051] | |
| ENSG00000184908 | CLCNKB | chloride voltage-gated channel Kb | 0.043516345 |
| | | [Source: HGNC Symbol; Acc: HGNC: 2027] | |
| ENSG00000197893 | NRAP | nebulin related anchoring protein | 0.043567821 |
| | | [Source: HGNC Symbol; Acc: HGNC: 7988] | |
| ENSG00000169126 | ARMC4 | armadillo repeat containing 4 | 0.043632647 |
| | | [Source: HGNC Symbol; Acc: HGNC: 25583] | |
| ENSG00000245248 | USP2-AS1 | USP2 antisense RNA 1 (head to head) | 0.043635087 |
| | | [Source: HGNC Symbol; Acc: HGNC: 48673] | |
| ENSG00000242866 | STRC | stereocilin | 0.043658722 |
| | | [Source: HGNC Symbol; Acc: HGNC: 16035] | |
| ENSG00000164393 | ADGRF2 | adhesion G protein-coupled receptor F2 | 0.044026611 |
| | | [Source: HGNC Symbol; Acc: HGNC: 18991] | |
| ENSG00000100033 | PRODH | proline dehydrogenase 1 | 0.044045719 |
| | | [Source: HGNC Symbol; Acc: HGNC: 9453] | |
| ENSG00000136352 | NKX2-1 | NK2 homeobox 1 | 0.044046233 |
| | | [Source: HGNC Symbol; Acc: HGNC: 11825] | |
| ENSG00000165566 | AMER2 | APC membrane recruitment protein 2 | 0.044155809 |
| | | [Source: HGNC Symbol; Acc: HGNC: 26360] | |
| ENSG00000163995 | ABLIM2 | actin binding LIM protein family member 2 | 0.044231879 |
| | | [Source: HGNC Symbol; Acc: HGNC: 19195] | |
| ENSG00000165495 | PKNOX2 | PBX/knotted 1 homeobox 2 | 0.044261202 |
| | | [Source: HGNC Symbol; Acc: HGNC: 16714] | |
| ENSG00000144115 | THNSL2 | threonine synthase like 2 | 0.044590058 |
| | | [Source: HGNC Symbol; Acc: HGNC: 25602] | |
| ENSG00000157214 | STEAP2 | STEAP2 metalloreductase | 0.044717969 |
| | | [Source: HGNC Symbol; Acc: HGNC: 17885] | |
| ENSG00000229240 | LINC00710 | long intergenic non-protein coding RNA 710 | 0.044849493 |
| | | [Source: HGNC Symbol; Acc: HGNC: 27386] | |
| ENSG00000168356 | SCN11A | sodium voltage-gated channel alpha subunit 11 | 0.044881812 |
| | | [Source: HGNC Symbol; Acc: HGNC: 10583] | |
| ENSG00000130508 | PXDN | peroxidasin | 0.044899327 |
| | | [Source: HGNC Symbol; Acc: HGNC: 14966] | |
| ENSG00000166444 | ST5 | suppression of tumorigenicity 5 | 0.044899327 |
| | | [Source: HGNC Symbol; Acc: HGNC: 11350] | |
| ENSG00000140600 | SH3GL3 | “SH3 domain containing GRB2 like 3, endophilin A3 | 0.045237232 |
| | | [Source: HGNC Symbol; Acc: HGNC: 10832]” | |
| ENSG00000214181 | NA | NA | 0.045237232 |
| ENSG00000144681 | STAC | SH3 and cysteine rich domain | 0.045389235 |
| | | [Source: HGNC Symbol; Acc: HGNC: 11353] | |
| ENSG00000166863 | TAC3 | tachykinin 3 | 0.045474144 |
| | | [Source: HGNC Symbol; Acc: HGNC: 11521] | |
| ENSG00000169436 | COL22A1 | collagen type XXII alpha 1 chain | 0.045474144 |
| | | [Source: HGNC Symbol; Acc: HGNC: 22989] | |
| ENSG00000172137 | CALB2 | calbindin 2 | 0.045474144 |
| | | [Source: HGNC Symbol; Acc: HGNC: 1435] | |
| ENSG00000223566 | TNRC18P2 | trinucleotide repeat containing 18 pseudogene 2 | 0.045474144 |
| | | [Source: HGNC Symbol; Acc: HGNC: 34014] | |
| ENSG00000175267 | VWA3A | von Willebrand factor A domain containing 3A | 0.045495146 |
| | | [Source: HGNC Symbol; Acc: HGNC: 27088] | |
| ENSG00000175267 | VWA3A | von Willebrand factor A domain containing 3A | 0.045495146 |
| | | [Source: HGNC Symbol; Acc: HGNC: 27088] | |
| ENSG00000183780 | SLC35F3 | solute carrier family 35 member F3 | 0.045495146 |
| | | [Source: HGNC Symbol; Acc: HGNC: 23616] | |
| ENSG00000228983 | SLC47A1P1 | solute carrier family 47 member 1 pseudogene 1 | 0.045495146 |
| | | [Source: HGNC Symbol; Acc: HGNC: 51849] | |
| ENSG00000269332 | GOLGA2P9 | golgin A2 pseudogene 9 | 0.045495146 |
| | | [Source: HGNC Symbol; Acc: HGNC: 49921] | |
| ENSG00000081800 | SLC13A1 | solute carrier family 13 member 1 | 0.045528908 |
| | | [Source: HGNC Symbol; Acc: HGNC: 10916] | |
| ENSG00000155816 | FMN2 | formin 2 | 0.045528908 |
| | | [Source: HGNC Symbol; Acc: HGNC: 14074] | |
| ENSG00000091137 | SLC26A4 | solute carrier family 26 member 4 | 0.045601783 |
| | | [Source: HGNC Symbol; Acc: HGNC: 8818] | |
| ENSG00000129990 | SYT5 | synaptotagmin 5 | 0.045601783 |
| | | [Source: HGNC Symbol; Acc: HGNC: 11513] | |
| ENSG00000173702 | MUC13 | “mucin 13, cell surface associated | 0.045601783 |
| | | [Source: HGNC Symbol; Acc: HGNC: 7511]” | |
| ENSG00000116176 | TPSG1 | tryptase gamma 1 | 0.045645845 |
| | | [Source: HGNC Symbol; Acc: HGNC: 14134] | |
| ENSG00000250420 | AACSP1 | acetoacetyl-CoA synthetase pseudogene 1 | 0.045645845 |
| | | [Source: HGNC Symbol; Acc: HGNC: 18226] | |
| ENSG00000104055 | TGM5 | transglutaminase 5 | 0.045691236 |
| | | [Source: HGNC Symbol; Acc: HGNC: 11781] | |
| ENSG00000109101 | FOXN1 | forkhead box N1 | 0.045781 |
| | | [Source: HGNC Symbol; Acc: HGNC: 12765] | |
| ENSG00000131386 | GALNT15 | polypeptide N-acetylgalactosaminyltransferase 15 | 0.045798489 |
| | | [Source: HGNC Symbol; Acc: HGNC: 21531] | |
| ENSG00000183016 | NA | NA | 0.045937358 |
| ENSG00000248746 | ACTN3 | actinin alpha 3 (gene/pseudogene) | 0.045937358 |
| | | [Source: HGNC Symbol; Acc: HGNC: 165] | |
| ENSG00000259010 | | | 0.04595318 |
| ENSG00000156687 | UNC5D | unc-5 netrin receptor D | 0.046099925 |
| | | [Source: HGNC Symbol; Acc: HGNC: 18634] | |
| ENSG00000213864 | EEF1B2P2 | eukaryotic translation elongation factor 1 beta | 0.046129239 |
| | | 2 pseudogene 2 | |
| | | [Source: HGNC Symbol; Acc: HGNC: 3209] | |
| ENSG00000143107 | FNDC7 | fibronectin type III domain containing 7 | 0.046229298 |
| | | [Source: HGNC Symbol; Acc: HGNC: 26668] | |
| ENSG00000230615 | | | 0.046269835 |
| ENSG00000184227 | ACOT1 | acyl-CoA thioesterase 1 | 0.046363122 |
| | | [Source: HGNC Symbol; Acc: HGNC: 33128] | |
| ENSG00000118194 | TNNT2 | “troponin T2, cardiac type | 0.046453265 |
| | | [Source: HGNC Symbol; Acc: HGNC: 11949]” | |
| ENSG00000172995 | ARPP21 | cAMP regulated phosphoprotein 21 | 0.046453265 |
| | | [Source: HGNC Symbol; Acc: HGNC: 16968] | |
| ENSG00000156103 | MMP16 | matrix metallopeptidase 16 | 0.04649564 |
| | | [Source: HGNC Symbol; Acc: HGNC: 7162] | |
| ENSG00000164904 | ALDH7A1 | aldehyde dehydrogenase 7 family member A1 | 0.04649564 |
| | | [Source: HGNC Symbol; Acc: HGNC: 877] | |
| ENSG00000224743 | TEX26-AS1 | TEX26 antisense RNA 1 | 0.04649564 |
| | | [Source: HGNC Symbol; Acc: HGNC: 42784] | |
| ENSG00000185823 | NPAP1 | nuclear pore associated protein 1 | 0.04652545 |
| | | [Source: HGNC Symbol; Acc: HGNC: 1190] | |
| ENSG00000018607 | ZNF806 | zinc finger protein 806 | 0.046600673 |
| | | [Source: HGNC Symbol; Acc: HGNC: 33228] | |
| ENSG00000179270 | C2orf71 | chromosome 2 open reading frame 71 | 0.046600673 |
| | | [Source: HGNC Symbol; Acc: HGNC: 34383] | |
| ENSG00000186862 | PDZD7 | PDZ domain containing 7 | 0.046710668 |
| | | [Source: HGNC Symbol; Acc: HGNC: 26257] | |
| ENSG00000227525 | RPL7P6 | ribosomal protein L7 pseudogene 6 | 0.046989116 |
| | | [Source: HGNC Symbol; Acc: HGNC: 32430] | |
| ENSG00000236229 | VEZF1P1 | vascular endothelial zinc finger 1 pseudogene 1 | 0.047105132 |
| | | [Source: HGNC Symbol; Acc: HGNC: 32320] | |
| ENSG00000171564 | FGB | fibrinogen beta chain | 0.047251427 |
| | | [Source: HGNC Symbol; Acc: HGNC: 3662] | |
| ENSG00000257175 | | | 0.047316621 |
| ENSG00000248713 | LOC285556 | “Homo sapiens uncharacterized mRNA. | 0.047420932 |
| | | [Source: RefSeq mRNA; Acc: NM_001354435]” | |
| ENSG00000102287 | GABRE | gamma-aminobutyric acid type A receptor | 0.047603565 |
| | | epsilon subunit | |
| | | [Source: HGNC Symbol; Acc: HGNC: 4085] | |
| ENSG00000150086 | NA | NA | 0.0476766 |
| ENSG00000168959 | GRM5 | glutamate metabotropic receptor 5 | 0.0476766 |
| | | [Source: HGNC Symbol; Acc: HGNC: 4597] | |
| ENSG00000184304 | PRKD1 | protein kinase D1 | 0.0476766 |
| | | [Source: HGNC Symbol; Acc: HGNC: 9407] | |
| ENSG00000204055 | | | 0.047795615 |
| ENSG00000164122 | ASB5 | ankyrin repeat and SOCS box containing 5 | 0.047913021 |
| | | [Source: HGNC Symbol; Acc: HGNC: 17180] | |
| ENSG00000123977 | DAW1 | dynein assembly factor with WD repeats 1 | 0.047973 |
| | | [Source: HGNC Symbol; Acc: HGNC: 26383] | |
| ENSG00000156413 | FUT6 | fucosyltransferase 6 | 0.047988989 |
| | | [Source: HGNC Symbol; Acc: HGNC: 4017] | |
| ENSG00000101276 | SLC52A3 | solute carrier family 52 member 3 | 0.048129781 |
| | | [Source: HGNC Symbol; Acc: HGNC: 16187] | |
| ENSG00000168079 | SCARA5 | scavenger receptor class A member 5 | 0.048129781 |
| | | [Source: HGNC Symbol; Acc: HGNC: 28701] | |
| ENSG00000254561 | | | 0.048129781 |
| ENSG00000223949 | ROR1-AS1 | ROR1 antisense RNA 1 | 0.048194625 |
| | | [Source: HGNC Symbol; Acc: HGNC: 40508] | |
| ENSG00000204335 | SP5 | Sp5 transcription factor | 0.048312303 |
| | | [Source: HGNC Symbol; Acc: HGNC: 14529] | |
| ENSG00000204241 | | | 0.04840783 |
| ENSG00000099625 | CBARP | CACN beta subunit associated regulatory protein | 0.048411296 |
| | | [Source: HGNC Symbol; Acc: HGNC: 28617] | |
| ENSG00000143450 | OAZ3 | ornithine decarboxylase antizyme 3 | 0.048617065 |
| | | [Source: HGNC Symbol; Acc: HGNC: 8097] | |
| ENSG00000015520 | NPC1L1 | NPC1 like intracellular cholesterol transporter 1 | 0.048746819 |
| | | [Source: HGNC Symbol; Acc: HGNC: 7898] | |
| ENSG00000188162 | OTOG | otogelin | 0.048746819 |
| | | [Source: HGNC Symbol; Acc: HGNC: 8516] | |
| ENSG00000125492 | BARHL1 | BarH like homeobox 1 | 0.048820483 |
| | | [Source: HGNC Symbol; Acc: HGNC: 953] | |
| ENSG00000145832 | SLC25A48 | solute carrier family 25 member 48 | 0.048934402 |
| | | [Source: HGNC Symbol; Acc: HGNC: 30451] | |
| ENSG00000185686 | PRAME | preferentially expressed antigen in melanoma | 0.048934402 |
| | | [Source: HGNC Symbol; Acc: HGNC: 9336] | |
| ENSG00000229147 | SMPD4P2 | sphingomyelin phosphodiesterase 4 pseudogene 2 | 0.049012128 |
| | | [Source: HGNC Symbol; Acc: HGNC: 39674] | |

### REFERENCES

1. Steinman L Immunology of relapse and remission in multiple sclerosis. Annu Rev Immunol 2014; 32:257-81.

2. Fava A, Petri M. Systemic lupus erythematosus: Diagnosis and clinical management. J Autoimmun 2019; 96:1-13.

3. Braun J, Wei B. Body traffic: ecology, genetics, and immunity in inflammatory bowel disease. Annu Rev Pathol 2007; 2:401-29.

4. Braun J, Baraliakos X, Listing J, et al. Differences in the incidence of flares or new onset of inflammatory bowel diseases in patients with ankylosing spondylitis exposed to therapy with anti-tumor necrosis factor alpha agents. Arthritis Rheum 2007; 57:639-47.

5. Sellam J, Marion-Thore S, Dumont F, et al. Use of whole-blood transcriptomic profiling to highlight several pathophysiologic pathways associated with response to rituximab in patients with rheumatoid arthritis: data from a randomized, controlled, open-label trial. Arthritis Rheumatol 2014; 66:2015-25.

6. Sanayama Y, Ikeda K, Saito Y, et al. Prediction of therapeutic responses to tocilizumab in patients with rheumatoid arthritis: biomarkers identified by analysis of gene expression in peripheral blood mononuclear cells using genome-wide DNA microarray. Arthritis Rheumatol 2014; 66:1421-31.

7. Tanino M, Matoba R, Nakamura S, et al. Prediction of efficacy of anti-TNF biologic agent, infliximab, for rheumatoid arthritis patients using a comprehensive transcriptome analysis of white blood cells. Biochem Biophys Res Commun 2009; 387:261-5.

8. Teixeira V H, Olaso R, Martin-Magniette M L, et al. Transcriptome analysis describing new immunity and defense genes in peripheral blood mononuclear cells of rheumatoid arthritis patients. PLoS One 2009; 4:e6803.

9. Zhang Y J, loerger T R, Huttenhower C, et al. Global assessment of genomic regions required for growth in Mycobacterium tuberculosis. PLoS Pathog 2012; 8:e1002946.

10. Aletaha D, Neogi T, Silman A J, et al. 2010 Rheumatoid arthritis classification criteria: an American College of Rheumatology/European League Against Rheumatism collaborative initiative. Arthritis Rheum 2010; 62:2569-81.

11. Aletaha D, Neogi T, Silman A J, et al. 2010 rheumatoid arthritis classification criteria: an American College of Rheumatology/European League Against Rheumatism collaborative initiative. Ann Rheum Dis 2010; 69:1580-8.

12. Pincus T, Swearingen C J, Bergman M, Yazici Y. RAPID3 (Routine Assessment of Patient Index Data 3), a rheumatoid arthritis index without formal joint counts for routine care: proposed severity categories compared to disease activity score and clinical disease activity index categories. J Rheumatol 2008; 35:2136-47.

13. Robison E H, Mondala T S, Williams A R, Head S R, Salomon D R, Kurian S M. Whole genome transcript profiling from fingerstick blood samples: a comparison and feasibility study. BMC Genomics 2009; 10:617.

14. Ritchie M E, Phipson B, Wu D, et al. limma powers differential expression analyses for RNA-sequencing and microarray studies. Nucleic Acids Res 2015; 43:e47.

15. Fischer D S, Theis F J, Yosef N. Impulse model-based differential expression analysis of time course sequencing data. Nucleic Acids Res 2018; 46:e119.

16. Bourgon R, Gentleman R, Huber W. Independent filtering increases detection power for high-throughput experiments. Proc Natl Acad Sci USA 2010; 107:9546-51.

17. Monaco G, Lee B, Xu W, et al. RNA-Seq Signatures Normalized by mRNA Abundance Allow Absolute Deconvolution of Human Immune Cell Types. Cell Rep 2019; 26:1627-40 e7.

18. Newman A M, Steen C B, Liu C L, et al. Determining cell type abundance and expression from bulk tissues with digital cytometry. Nat Biotechnol 2019; 37:773-82.

19. Love M I, Huber W, Anders S. Moderated estimation of fold change and dispersion for RNA-seq data with DESeq2. Genome Biol 2014; 15:550.

20. Zhang F, Wei K, Slowikowski K, et al. Defining inflammatory cell states in rheumatoid arthritis joint synovial tissues by integrating single-cell transcriptomics and mass cytometry. Nat Immunol 2019; 20:928-42.

21. Mizoguchi F, Slowikowski K, Wei K, et al. Functionally distinct disease-associated fibroblast subsets in rheumatoid arthritis. Nat Commun 2018; 9:789.

22. Croft AP, Campos J, Jansen K, et al. Distinct fibroblast subsets drive inflammation and damage in arthritis. Nature 2019; 570:246-51.

23. Lefevre S, Knedla A, Tennie C, et al. Synovial fibroblasts spread rheumatoid arthritis to unaffected joints. Nat Med 2009; 15:1414-20.

24. Lu D R, McDavid A N, Kongpachith S, et al. T Cell-Dependent Affinity Maturation and Innate Immune Pathways Differentially Drive Autoreactive B Cell Responses in Rheumatoid Arthritis. Arthritis Rheumatol 2018; 70:1732-44.

25. Mikuls T R, Payne J B, Yu F, et al. Periodontitis and Porphyromonas gingivalis in patients with rheumatoid arthritis. Arthritis Rheumatol 2014; 66:1090-100.

26. Konig M F, Abusleme L, Reinholdt J, et al. Aggregatibacter actinomycetemcomitans-induced hypercitrullination links periodontal infection to autoimmunity in rheumatoid arthritis. Sci Transl Med 2016; 8:369ra176.

27. Eriksson K, Nise L, Alfredsson L, et al. Seropositivity combined with smoking is associated with increased prevalence of periodontitis in patients with rheumatoid arthritis. Ann Rheum Dis 2018; 77:1236-8.

### EXAMPLE 2

The system and methods detailed herein and in Example 1 provide a framework and approach to evaluating individuals at risk of disease, for assessing disease progression, or for identifying markers of more severe disease or metastasis. The availability of a system and method to isolate and analyze high quality RNA which validly identifies RNA, including RNA changes in vivo and new or altered RNAs such as those of different cellular components or of infectious agents, from patient self-collected samples, including small volume blood samples (finger stick), provides a means to monitor and assess various disease or infectious conditions or scenarios particularly where traditional blood sampling is not warranted, feasible or practical. Applications for evaluation and monitoring of cancer patients, including prior to and following treatment or remission, as well as patients with relapsing or remitting diseases such as multiple sclerosis, Crohn's disease, etc are contemplated. Further, the system and methods can be applied and implemented in infectious disease, including in viral diseases that affect large populations, either seasonally or in unanticipated circumstances. Those at risk of infection or who are presumed or determined to be infected can be evaluated to assess the RNA response and RNA indicators of disease, characterize predictors or markers of susceptibility or disease severity, and identify targets for treatment or modulation. For example, more precise and marker-based knowledge and understanding of influenza virus infection and susceptibility could reduce the effects of seasonal influenza on individuals and the health care system. Further, the recent outbreak of new coronavirus SARS-COV2 and the COVID-19 pandemic underscores an imminent need for a system, method and approach as provided herein.

Coronaviruses are a family of viruses that can cause illnesses such as the common cold, severe acute respiratory syndrome (SARS) and Middle East respiratory syndrome (MERS). In late 2019, a new coronavirus, severe acute respiratory syndrome coronavirus 2 (SARS-CoV-2), was identified as the cause of a COVID-19 disease outbreak that originated in China. In March 2020, the World Health Organization (WHO) declared the COVID-19 outbreak a pandemic. By early April, the worldwide number of confirmed COVID-19 cases were nearly 1 5 million, with over 400,000 in the U.S., over 135,000 cases in each of Italy and Spain, over 100,000 cases in Germany and over 80,000 reported in China. Deaths worldwide were over 80,000. No accepted or approved treatments or vaccines are available.

Signs and symptoms of COVID-19 appear 2 to 14 days after exposure and can include fever, cough, shortness of breath or difficulty breathing, as well as tiredness, aches, runny nose and sore throat. Some people experience the loss of smell or taste. People who are older or who have existing chronic medical conditions, such as heart disease, lung disease or diabetes, or who have compromised immune systems may be at higher risk of serious illness, similar to what is seen with other respiratory illnesses, such as influenza.

The severity of COVID-19 symptoms can range from very mild to severe and some people may have no symptoms at all. In fact, studies have shown that a significant portion of individuals with coronavirus lack symptoms (“asymptomatic”) and that even those who eventually develop symptoms (“pre-symptomatic”) can transmit the virus to others before showing symptoms (Li R et al Science 10.1126/science.abb3221(2020); Rothe C et al (2020) New Engl J Med 382(10):970-971; Zou L et al (2010) New Engl J Med 382(12)1177-1179). Therefore, the virus can spread between people interacting in close proximity—for example, speaking, coughing, or sneezing—even if those people are not exhibiting symptoms.

In the United States, nearly one-third of COVID-19 disease cases are 6 or older and patients over 65 account for nearly half of hospitalizations and a significant majority of deaths, according to CDC reports. Nonetheless, about 20% of infected people ages 20-44 are hospitalized, demonstrating that this is not just a disease of older adults. Important outstanding questions exist as to the underlying biological vulnerability of older individuals and how do preexisting conditions or illnesses exacerbate COVID-19. Also, it would be helpful to have indicators for those patients who will develop more significant or severe disease, so they can be managed or triaged differently or more aggressively.

RNA monitoring and longitudinal genomics in accordance with the system and methods provided herein, including as set out in Example 1, provides an approach to isolate, identify and evaluate RNAs in individuals exposed to or at risk of virus infection, such as coronavirus infection, such as with SARS-COV2, or patients infected with the virus and diagnosed for COVID-19. The systems and methods could be implemented in individuals post-vaccine also to evaluate RNA, protein and cellular response(s). Finger stick collection of small blood samples as described herein may be implemented by regular collection at home, at or in hospital, by medical care workers or personnel, or in isolation or quarantine. This permits monitoring of the infection, including viral RNA, disease, RNA response, RNA alterations, including as an indicator of cellular response as described above and in Example 1. The availability of high quality RNA from prospective and retrospective sampling will facilitate an understanding of infection and disease, including in influenza, coronavirus, or instances of other known or unknown infectious agents, including new variants, as well as the body's response to disease and susceptibility to disease aspects. Collection of standard venipuncture samples puts health care workers at risk and is unduly invasive and difficult for patients and individuals already suffering or in stressful and demanding situations and conditions.

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A method for RNA profiling and analysis of small volume samples from a patient or individual comprising:
(a) obtaining one or more small volume sample self-collected by the patient or individual or by a non-medical person, wherein the sample is collected in or otherwise combined with an RNA stabilization solution whereby cells in the sample are lysed and RNA is stabilized; and
(b) isolating RNA using a process adapted for small volume samples wherein the amount of any and all solutions or buffers utilized are reduced and adjusted for small volume samples;
wherein the RNA is of sufficient quality and quantity for whole transcriptome analysis and transcriptomic profiling through RNA sequencing (RNAseq).

2. The method of claim 1 wherein the RNA is isolated using a process comprising:
(a) contacting the sample with a protease to form a protease treated small volume sample;
(b) contacting the protease treated sample with an ethanol or salt solution forming a precipitate containing the RNA, wherein the precipitate containing the RNA is then resuspended in a buffer or solution, or contacting the protease treated sample with an organic extraction solution, forming a solution having an aqueous phase containing the RNA and an organic phase;
(c) contacting the resuspended precipitate containing the RNA or the aqueous phase containing the RNA with DNAse to form a DNAse-treated resuspended precipitate or DNAse-treated aqueous phase;
(d) binding the RNA to a silica based solid phase or column by contacting the resuspended precipitate or aqueous phase with said silica based solid phase; and
(e) eluting the RNA from the silica based solid phase comprising contacting the silica based solid phase with a solution or buffer to provide isolated RNA;
wherein all buffer and solution volumes are reduced and adjusted for small volume samples.

3. The method of claim 2 wherein between steps (b) and (c), the resuspended precipitate containing the RNA or the aqueous phase containing the RNA is contacted with a solution or column to remove residual sample cell debris and/or to homogenize the sample cell lysate.

4. The method of claim 1, wherein the RNA is isolated using a process comprising:
(a) contacting the sample with an RNA stabilization solution, wherein the solution has capability to lyse cells and inactivate adventitious agents;
(b) optionally further contacting the sample with a salt, a reducing agent, and/or a detergent;
(c) contacting the solution contacted sample of (a) or (b) with silica, silica based solid phase or carboxylated magnetic beads which bind RNA and seves to purify the RNA from other components in the sample; and
(d) eluting the RNA from the silica or silica based solid phase or the magnetic beads comprising contacting the silica, silica based solid phase or magnetic beads with a solution or buffer to provide isolated RNA;
wherein all buffer and solution volumes are reduced and adjusted for small volume samples.

5. The method of claim 1, wherein the sample is a small volume blood sample.

6. The method of claim 1, wherein the small volume blood sample is collected via fingerstick.

7. The method of claim 1, wherein the sample volume is less than 500 μl, less than 300 μl, less than 250 μl, about 200-300 μl, less than 200 μl, about 100-300 μl, about 150-300 μl, about 100-250 μl, about 50-300 μl, less than 100 μl, less than 50 μl, less than 25 μl, 10 μl or less.

8. The method of claim 1, wherein buffer and solution volumes are reduced to 20-40% or 20-30% or about 25% of those utilized for isolation of RNA from a standard venipuncture blood sample.

9. The method of claim 2, wherein said protease is proteinase K.

10. The method of claim 1, further comprising sequencing the RNA.

11. The method of claim 10, wherein abundant RNA species or RNA species not of interest are removed prior to sequencing.

12. The method of claim 11, wherein globin mRNA, ribosomal RNA(s) or species specific RNAs are removed prior to sequencing.

13. The method of claim 1, wherein the patient or individual has a disease or infection or is at risk of or suspected of disease or infection.

14. The method of claim 1 for longitudinal screening by RNA profiling and analysis of small volume samples from one or more patient or individual, wherein the patient or individual has a disease or infection or is at risk of or suspected of disease or infection.

15. The method of claim 14, wherein small volume samples are collected via fingerstick in series or in regular or designated increments of hours, days, weeks or months.

16. The method of claim 15, wherein small volume blood samples are collected via fingerstick in series or in regular or designated increments of hours, days, weeks or months.

17. A system or kit for RNA profiling and analysis of small volume samples from a patient or individual comprising:
(a) a means for self-collection of a small volume sample by the patient or individual or by a non-medical person, comprising a lancet, swab or receptable for a wash, spit or aspirate;
(b) a tube or receptacle for receiving the small volume sample on collection and containing a volume of RNA stabilization solution whereby cells in the sample are lysed and RNA is stabilized; and
(c) one or more appropriate label(s) for designating the name or identity of the patient or individual, date of sample collection and time of sample collection.

18. The system or kit of claim 17, further comprising an envelope or mailing container for shipment of the sample to a laboratory or facility for RNA isolation and analysis.

19. The system or kit of claim 17, for longitudinal RNA profiling and analysis of multiple small volume samples collected in series from a patient or individual over days, weeks or months comprising:
(a) a set of numerous means for self-collection of individual small volume samples by the patient or individual or by a non-medical person, each comprising a lancet, swab or receptable for a wash, spit or aspirate;
(b) a set of numerous tubes or receptacles each individually for receiving a small volume sample on collection and containing a volume of RNA stabilization solution whereby cells in the sample are lysed and RNA is stabilized;
(c) numerous appropriate label(s) for designating the name or identity of the patient or individual, date of sample collection and time of sample collection; and
(d) numerous envelopes or mailing containers for shipment of each sample or several samples to a laboratory or facility for RNA isolation and analysis.

20. The system or kit of claim 17, wherein the volume of RNA stabilization solution is less than 1 ml, about 500 μl or less, about 300 μl or less, about 200-300 μl, about 250 μl, less than 200 μl, less than 100 μl, less than 50 μl, less than 25 μl, or 10 μl or less.

21. The system or kit of claim 17, wherein the tube or receptacle for receiving the small volume sample and containing RNA stabilization solution has a total volume capacity of 1.5 ml or less, 1.2 ml or less, or 1 ml or less.
